(19) United States
(12) Patent Application Publication
(10) Pub. No.: US 2006/0058383 A1
(43) Pub. Date: Mar. 16, 2006

(54) Propyl 3-bromo-2-oxopropionate and derivatives as novel anticancer agents

(76) Inventors: Peng Huang, Bellaire, TX (US); Michael J. Keating, Houston, TX (US); Ruihau Xu, Guangzhou (CN)

Correspondence Address: Fulbright & Jaworski L.L.P.;Fulbright Tower, Suite 5100, 1301 McKinney, Houston, TX, 77010-3095, US

(21) Appl. No.: 11/192,281

(22) Filed: Jul. 28, 2005

Related U.S. Application Data
(60) Provisional application No. 60/591,643, filed on Jul. 28, 2004.

(51) Int. Cl. A61K 31/22; A61K 31/19

(52) U.S. Cl. 514/546; 514/557

(57) ABSTRACT

The present invention is directed to compositions that inhibit glycolysis, perferentially in cancer. Specifically, the anticancer compositions comprise 3-halo-2-oxopropionate and its derivatives, such as ester derivatives. However, in specific embodiments, the anticancer composition is sodium 3-halo-2-oxopropionate, such as sodium 3-bromo-2-oxopropionate and a stabilizing agent, such as carbonic acid. In particular embodiments, the compositions of the present invention further comprise a metabolic intermediate for normal cells to utilize in a pathway for an alternate energy source, thereby providing protection to normal cells. In other embodiments, the 3-halo-2-oxopropionate or its ester derivative is used in combination with an additional cancer therapy, such as radiation and/or a drug.

The present invention claims priority to U.S. Provisional Patent Application No. 60/591,643, filed Jul. 29, 2004, which is incorporated by reference herein in its entirety.

### FIELD OF THE INVENTION

The present invention relates to the fields of cell biology, pharmacology and cancer therapy. In particular, the invention relates to the field of glycolysis inhibitors for cancer therapeutics.

### BACKGROUND OF THE INVENTION

Compared to normal cells, cancer cells generally exhibit increased glycolysis and are more dependent on this metabolic pathway for ATP generation to maintain their energy supply (known as the Warburg effect). The dependency on glycolysis is attributed in part to mitochondria malfunction (respiration injury) associated with mitochondrial DNA mutations and oncogenic transformation in cancer cells and to hypoxic conditions in the tumor tissues. In contrast, normal cells with competent mitochondria can generate ATP efficiently through oxidative phosphorylation (respiration) and can use alternative energy sources when glycolysis is inhibited. Furthermore, many human cancers, including most solid tumors, grow in a tissue environment where oxygen supply is severely limited or absent, a condition known as hypoxia due in part to large tumor mass with relatively limited blood supply. Under hypoxic conditions, cancer cells use the glycolytic pathway to generate ATP without using oxygen. This metabolic adaptation further renders the cancer cells dependent on glycolysis for meeting their energy requirement. Thus, the difference between normal and cancer cells in their energy metabolism and dependency in glycolysis, due either to mitochondrial defect or to a hypoxic environment, provides a biochemical basis to preferentially kill cancer cells by inhibition of glycolysis.

The present invention relates, in general, to compositions and methods aimed at effectively treating cancer cells with inhibitors of glycolysis. Exemplary glycolysis inhibitors include compositions related to pyruvate. For example, pyruvate derivates are described in U.S. Patent Application Publications U.S. 2003/0013656, U.S. 2003/0013847, US 2003/0013657, and U.S. 2003/0013846, particularly for treating conditions characterized by oxidative stress, such as neurodegenerative disorders, stroke, myocardial ischemia, asthma, and so forth.

Inactivation of brain glutamic decarboxylase by 3-bromopyruvate was described by Tunnicliff and Ngo (1978).

Inhibition of fatty acid synthesis for treatment of tumors is described in EP 0651 636B1.

U.S. Pat. No. 4,935,450 relates to treatment of malignant cells by administering an ATP-availability depressor agent for limiting the overall rate of ATP energy available to support malignant cell metabolism.

U.S. Pat. No. 6,472,378 regards pyrimidine nucleotide precursors, such as pyruvyluridine compounds, for treatment of mitochondrial diseases, including cancer.

WO 02/45720, WO 03/105862, and U.S. Patent Application Publication U.S. 2003/0139331 relate to cancer treatment by reducing intracellular energy and pyrimidines, particularly by administering a combination of an ATP-depleting agent at a concentration that depletes the ATP level to at least 15% of normal in cancer cells; a pyrimidine antagonist; and an anticancer agent to which the treated cancer is sensitive.

Ko et al. (2001) demonstrate inhibition of glycolytic rate and cell death in vitro for liver cancer cells treated with 3-bromopyruvate. Geschwind et al. (2002) and Harrison and Di Bisceglie (2003) describe systemic delivery of 3-bromopyruvate for the treatment of liver cancer and suppression of metastatic lung tumors in rabbits.

U.S. Patent Application Publication U.S. 2003/0087961 describes methods of treating tumors using inhibitors of ATP production, including 3-bromopyruvate. In specific embodiments the inhibitor is administered with a second agent, such as a chemotherapeutic agent or a scavenger compound.

U.S. Patent Application Publication U.S. 2003/0181393 regards glycolytic inhibitors for cancer treatment, including a 3-halo-pyruvate.

U.S. Pat. No. 6,670,330 concerns six categories of glycolytic inhibitors for tumor treatment, particularly to increase the efficacy of chemotherapeutic and radiation regimens. In specific embodiments, 3-halopyruvate may be utilized.

In view of continual needs to provide cancer therapies, there exists a need for additional chemotherapeutic agents, particularly directed to the effective cancer-specific target of glycolysis inhibition. The need is provided by the compositions and methods described herein.

### BRIEF SUMMARY OF THE INVENTION

The present invention is directed to a system and method that regard a novel anticancer composition that exploits the requirement of cancer cells to rely on glycolysis for ATP generation to maintain their energy supply. In a particular aspect of the invention, the anticancer composition is Glycolycin, which comprises a derivative of 3-halo-2-oxopropionate, such as an ester derivative (also referred to as 3-halopyruvate ester), or a salt of 3-halo-2 oxopropionate stabilized by a stabilizing agent such as sodium carbonate or sodium bicarbonate (such as sodium 3-halo-2-oxopropionate, which is also referred to as sodium 3-halopyruvate). In specific aspects, a Glycolycin composition is synthesized by proper esterification of 3-halo-2-oxopropionate with an alcohol, or by stabilization of 3-halo-2-oxopropionate with sodium carbonate or sodium bicarbonate. The ester of 3-halo-2-oxopropionate is propyl 3-bromo-2-oxopropionate (which may be referred to herein as Glycolycin, also referred to as 3-bromo-2-oxopropionic acid propyl ester or propyl 3-bromopyruvate), for example, although it may also be 3-fluoro-2-oxopropionic acid propyl ester; 3-chloro-2-oxopropionic acid propyl ester; 3-iodo-2-oxopropionic acid propyl ester.

An ester of 3-halo-2-oxopropionate may also be 3-bromo-2-oxopropionic acid ethyl ester (which may be referred to herein as E-Glycolycin, also referred to as ethyl 3-bromo-2-oxopropionate or ethyl 3-bromopyruvate); 3-fluoro-2-oxopropionic acid ethyl ester; 3-chloro-2-oxopropionic acid ethyl ester; or 3-iodo-2-oxopropionic acid ethyl ester. An ester of 3-halo-2-oxopropionate may also be 3-bromo-2-oxopropionic acid methyl ester (which may be referred to herein as M-Glycolycin, also referred to as methyl 3-bromo-2-oxopropionate or methyl 3-bromopyruvate); 3-fluoro-2-oxopropionic acid methyl ester; 3-chloro-2-oxopropionic acid methyl ester; or 3-iodo-2-oxopropionic acid methyl ester. An ester of 3-halo-2-oxopropionate may also be 3-bromo-2-oxopropionic acid pentyl ester (which may be referred to herein as P-Glycolycin). Table 1 provides a listing of particular exemplary Glycolycin compounds.

TABLE 1
Glycolycin and Exemplary Derivative Compounds

| | |
|---|---|
| Glycolycin | propyl 3-halo-2-oxopropionate; 3-halo-2- |
| | oxopropionic acid propyl ester; propyl 3- |
| | halopyruvate |
| M-Glycolycin | 3-halo-2-oxopropionic acid methyl ester; |
| | methyl 3-halo-2-oxopropionate; methyl 3- |
| | halopyruvate |
| E-Glycolycin | 3-halo-2-oxopropionic acid ethyl ester; ethyl 3- |
| | halo-2-oxopropionate; ethyl 3-halopyruvate |
| S-Glycolycin | a salt of 3-halo-2 oxopropionate stabilized by a |
| | stabilizing agent such as sodium carbonate or |
| | sodium bicarbonate |
| P-Glycolycin | Pentyl 3-bromo-2-oxopropionate; 3-halo-2- |
| | oxopropionic acid pentyl esters; pentyl 3- |
| | halopyruvate |

In specific aspects of the invention, general classes of chemical agents that form esters with 3-halo-2-oxopropionate include alcohols of various carbons and hydroxyl groups, as long as they can react with the carboxyl of the 3-halo-2-oxopropionate to form an ester, which can then be hydrolyzed to release the active 3-halo-2-oxopropionate within the cells by esterases in the biological system. In an alternative embodiment, an esterase may be provided with the composition that may be delivered to the target cells. Targeting to specific cells may be achieved by any suitable means in the art, such as with cancer-recognizing antibodies. Administration of the Glycolycin composition and an esterase may be in any manner, but in specific embodiments it occurs sequentially or through separate administrations.

In particular aspects of the invention, the sodium 3-halo-2-oxopropionate is sodium 3-bromo-2-oxopropionate (also referred to as sodium 3-bromopyruvate), for example, although it may also be sodium 3-fluoro-2-oxopropionate, sodium 3-chloro-2-oxopropionate, or sodium 3-iodo-2-oxopropionate, in specific embodiments. The stabilizing agent may comprise carbonic acid, although alternative stabilizing agents may be used in addition or alternative to carbonic acid. For example, sodium bicarbonate may be utilized so long as it ultimately generates carbonic acid.

The inventors show that Glycolycin and its derivatives/analogs have superior pharmaceutical properties compared to other glycolytic inhibitors, and is able to effectively block glycolysis and cause a severe depletion of the cellular ATP pool and massive cell death, especially in cancer cells with increased dependency on glycolysis in a hypoxic environment or when mitochondrial respiration is defective. In other particular aspects of the invention, Glycolycin further comprises one or more components that provide, directly or indirectly, an alternate energy source to enhance the ability of normal cells to maintain appropriate energy requirements for cell survival in the event of inhibited glycolysis. The alternate energy source may be of any kind, but in particular aspects of the invention it is a metabolic intermediate, such as one that facilitates utilization by normal cells of pathways that produce energy, for example ATP. The pathways for alternative energy sources may be the TCA cycle and mitochondrial respiration, for example. In addition, the alternative energy sources may be metabolic intermediates of these pathways or precursors thereto, so as to enhance utilization of these pathways.

Particular examples of metabolic intermediates include glutamine, pyruvate, fatty acids and/or mixtures thereof. Thus, based on the mechanism of action of Glycolycin and the difference in energy metabolism between cancer and normal cells, appropriate components can be added to protect the normal cells from the toxicity of Glycolycin without significantly compromising its anticancer activity. This aspect of the invention encompasses an embodiment of drug formulations with increased therapeutic selectivity, and any of the Glycolycin derivatives described herein may be utilized in such a manner. In particular, the present inventors have designed two such exemplary mechanism-based formulations referred to herein as Glycolycin-G (Glycolycin and glutamine) and Glycolycin-P (Glycolycin and pyruvate).

In particular aspects of the invention, there are provided methods for production of Glycolycin and its derivatives. These production methods are based on the following chemical reaction principle:

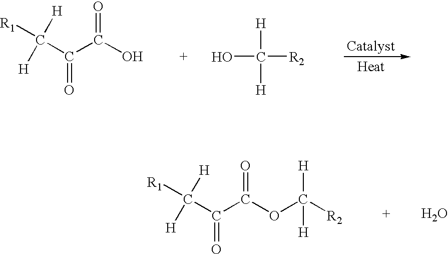

where R1 is a halogen (F, Cl, Br, or I, for example), and R2 may be a hydrogen atom (H) or a multi-carbon group in linear structure (—CH₃ or —CH₂—CH₃, or —CH₂—CH₂—CH₃, for example) or in ring structure (a benzol derivative, for example). The catalyst is a concentrated acid, such as hydrochloric acid (HCl) or sulfuric acid (H₂SO₄), for example. These production methods, in some embodiments and by example only, comprise starting materials including 3-bromo-2-oxopropionic acid (which is also referred to as 3-bromopyruvate), 1-propanol, hydrochloric acid, sodium carbonate, sodium bicarbonate, and water, such as double-distilled water. Specifically, the chemical reaction of 1-propanol and 3-bromo-2-oxopropionic acid in the presence of hydrochloric acid and proper temperature will produce 3-bromo-2-oxopropionic acid propyl ester (referred to as Glycolycin). In other embodiments, an alternative alcohol such as ethanol or methanol, or another compound with a reactive hydroxyl is utilized for the production of the respective 3-halopyruvate esters.

An alternative 3-halopyruvate may be utilized for production methods, such as 3-fluoropyruvate, 3-iodopyruvate, or 3-chloropyruvate, thereby generating, for example, 3-fluoro-2-oxopropionic acid propyl ester, 3-chloro-2-oxopropionic acid propyl ester, 3-iodo-2-oxopropionic acid propyl ester, 3-bromo-2-oxopropionic acid ethyl ester (E-Glycolycin), 3-. fluoro-2-oxopropionic acid ethyl ester, 3-chloro-2-oxopropionic acid ethyl ester, 3-iodo-2-oxopropionic acid ethyl ester, 3-bromo-2-oxopropionic acid methyl ester, 3-fluoro-2-oxopropionic acid methyl ester (M-Glycolycin), 3-chloro-2-oxopropionic acid methyl ester, or 3-iodo-2-oxopropionic acid methyl ester. In yet another aspect of the invention, stabilization of 3-halopyruvate, such as 3-bromopyruvate, 3-fluoropyruvate, 3-iodopyruvate, or 3-chloropyruvate, with sodium carbonate or sodium bicarbonate will generate, respectively, sodium 3-bromopyruvate (S-Glycolycin), sodium 3-fluoropyruvate, sodium 3-iodopyruvate, or sodium 3-chloropyruvate, and in specific embodiments these compounds benefit from a stabilizing agent, similar to that for 3-bromopyruvate.

Although in some aspects of the invention Glycolycin compositions are prepared by methods that chemically produce the desired composite product in its desired component ratio, in other embodiments the Glycolycin compositions are generated by an alternative manner. For example, the components of Glycolycin may be obtained by commercial means and mixed in a desired ratio to generate the chemical reactions leading to production of Glycolycin. The desired ratio may be any kind such that it provides a therapeutic effect. In specific embodiments, the ratio is preferably such that the molar amount of the alcohol (1-propanol, for example) is in excess over 3-halopyruvate to generate the chemical reactions leading to production of Glycolycin with favorable yield. In particular, it is found that the molar ratio of 3:1 or 2:1 for 1-propanol:3-bromopyruvate produces satisfactory chemical reaction and generate Glycolycin, using concentrated hydrochloric acid (HCl) as a catalyst. After the chemical reaction is completed, the excess alcohol (1-propanol, for example) can be readily removed by evaporation under low pressure (proper vacuum) at low temperature. In other specific embodiments, the carbonic acid component provides stability to the 3-bromo-2-oxoproprionate component, which comprises anti-cancer activity. Therapeutic benefits can comprise destruction of at least one cancer cell to providing amelioration one or more symptoms of cancer in an individual. In specific aspects of the invention, the ratio of sodium 3-halopyruvate to stabilizing agent is about 2:1, respectively.

In particular aspects of the invention, a Glycolycin composition comprising one or more alternate energy agents, such as metabolic intermediates from the tricarboxylic acid cycle (TCA) pathway, mitochondrial respiration, or both, is prepared. The alternate energy agents may be mixed with the reaction substrates to generate Glycolycin, or they may be mixed subsequent to its production.

In one particular aspect of the invention, there is a method for inhibiting glycolysis in any cell by delivering Glycolycin to the cell. In specific aspects, the method is further defined as inducing apoptosis and/or necrosis in the cell or inhibiting proliferation of the cell. In those embodiments where apoptosis is induced, the apoptosis may be of any kind, although in particular embodiments it is mediated by dephosphorylation of BAD, a molecule involved in the integration of apoptosis and glycolysis. Apoptotic cells may also show changes in the mitochondrial membrane permeability, release of cytochrome c to the cytosol, and/or activation of the caspases. In other embodiments where necrosis is induced, this form of cell death may be caused by various biochemical and molecular changes induced by Glycolycin, although in particular embodiments it is mediated by depletion of cellular ATP. In additional aspects, the cell in which glycolysis is inhibited is a cancer cell. Furthermore, the cells in which Glycolycin may be particularly useful are those having one or more mitochondrial defects due to one or more mutations in mitochondrial DNA, a frequent occurrence in cancer cells.

In another important aspect of the invention, the cancer cells in which Glycolycin may be particularly useful are those in a hypoxic environment, a condition frequently present in many human cancers, especially solid tumors. It is known in the art that under hypoxic conditions, cancer cells become less sensitive to radiation treatment and to certain anticancer agents. In specific embodiments, Glycolycin is particularly useful in the treatment of cancer in hypoxic conditions and in additional embodiments in overcoming drug resistance and radiation resistance, such as those associated with hypoxia.

In yet another aspect of the invention, the cancer cells in which Glycolycin is particularly useful are those expressing multi-drug resistance proteins, such as MDR and MRP, which use ATP as the energy source to pump drug molecules out of the cells and confer a multi-drug resistant phenotype. Inhibition of glycolysis and depletion of cellular ATP by Glycolycin deprives cancer cells of the energy source for the ATP-dependent drug pump, and thus re-sensitizes the cancer cells to anticancer agents. In a specific aspect, the present inventors demonstrated that an exemplary human leukemia cell line with a multi-drug resistant phenotype remains sensitive to Glycolycin.

The compositions and methods described herein are contemplated for any type of cancer. For example, the invention may be utilized for brain cancer, lung cancer, breast cancer, prostate cancer, pancreatic cancer, ovarian cancer, liver cancer, bone cancer, esophageal cancer, colon cancer, head and neck cancer, leukemia, lymphoma, melanoma, spleen cancer, cervical cancer, kidney cancer, and/or throat cancer. In particular aspects of the invention, the present inventors demonstrated that Glycolycin is effective against human leukemia cells and solid tumor cells, for example. This in vitro therapeutic activity has been demonstrated both in culture cell lines and in primary cancer cells isolated from patients, and in particular embodiments is contemplated for in vivo therapeutic purposes.

In a particular aspect of the invention, Glycolycin, or Glycolycin in combination with a metabolic intermediate, is utilized to treat cancer and/or to overcome drug resistance of a cancer, and in particular embodiments it is utilized in conjunction with or subsequent to another cancer therapy. The inventors demonstrate herein that Glycolycin maintains its activity against cancer cells resistant to other anticancer agents. More specifically, it is contemplated that Glycolycin in combinations with other anticancer agents or modalities enhance therapeutic activity and selectivity. Given that ATP generation through glycolysis is essential for cancer cells, it is less likely that cancer cells will develop resistance to Glycolycin.

In another aspect of the invention, Glycolycin is utilized in combination with ionizing radiation to kill cancer cells. Radiation kills cancer cells by damaging cellular DNA. However, in the presence of a sufficient ATP supply, cells may be able to repair the DNA damage and resist radiation at least to a certain degree. By inhibition of glycolysis and depletion of cellular ATP, Glycolysis is particularly useful in combination with radiotherapy to effectively treat cancer, in some embodiments of the invention. Such a favorable combinatory effect in killing cancer cells in vitro has been demonstrated by the current inventors.

In yet another aspect of the invention, Glycolycin is utilized in combination with other anticancer agents with DNA-damaging property to increase the effectiveness of killing cancer cells. Cancer cells are known to use various mechanisms to repair DNA damage induced by anticancer agents. These DNA repair processes are largely dependent on the presence of ATP as the energy source for the biochemical reactions. Insufficient supply of ATP would hinder DNA repair and thus enhance the cytotoxic effect of DNA-damaging agents. This invention provides that inhibition of glycolysis by Glycolycin results in a depletion of cellular ATP, and thus can be particularly useful in combination with DNA-damaging anticancer agents, such as doxorubicin, cisplatin, cyclophosphomide, and nucleoside analogs, for example. Such drug combinations with favorable anticancer activity in vitro have been demonstrated by the present inventors.

Another strategy to impact cancer cell energy metabolism is to target the regulatory mechanisms that affect the expression or functions of protein molecules that are directly or indirectly involved in nutrient metabolism. Recent studies have generated compelling evidence suggesting that the mTOR (mammalian target of rapamycin) pathway play important roles in nutrient uptake, regulation of energy metabolism and cell proliferation, and promoting cancer cell survival.9-12 The critical functions of mTOR have attracted a significant attention of the research community and pharmaceutical companies, and led to the development of a number of novel compounds that target the mTOR pathway. CCI-779, RAD001, and AP-23573 are examples of this class of compounds currently in clinical trials for cancer treatment (Raymond et al., 2004; Panwalkar et al., 2004). These new compounds, like their parental compound rapamycin, directly target mTOR and effectively affect the function of this pathway and cause alterations in cellular metabolism and survival signaling. Thus, in particular aspects, the invention provides an effect, such as a synergistic effect, between glycolycin and a drug, such as one that inhibits the mammalian target of rapamycin (mTOR) pathway, including rapamycin, CCI-779, RAD001, and AP-23573, for example.

In specific aspects of the invention, glycolycin is combined with an agent that makes it more dependent on glycolysis for generation of ATP, and therefore more sensitive to the action of glycolycin. For example, inhibitors of the mitochondrial respiratory chain may be used in combination with glycolycin so that cancer cells, under the influence of such inhibitors, are more dependent on glycolysis for generation of ATP, and thus more sensitive to the action of glycolycin. Examples of respiratory inhibitors that may be combined with glycolycin for treatment of cancer include rotenone and arsenic trioxide.

The methods and compositions of the present invention provide advantages over compositions known in the art. Glycolycin, with its unique chemical characteristics, has superior pharmaceutical properties over known compositions, including improved stability, increased penetration into the cells, ease of synthesis, and cost-effectiveness for scale-up production, for example. It is of a particular advantage that Glycolycin is more stable and chemically less polarized than currently available glycolytic inhibitors with a similar mechanism of action, and it is readily permeable through the cellular membranes. Once inside the cells, for example, the exemplary Glycolycin derivative is cleaved by the cellular enzyme esterase, generating two hydrolytic products, 3-bromopyruvate and 1-propanol. The intracellular 3-bromopyruvate is the active component that inhibits glycolysis leading to ATP depletion and killing of the cancer cells that rely on glycolysis. The second hydrolytic product, 1-propanol, can be further converted by alcohol dehydrogenase to propionic acid, which is in turn converted to propionyl CoA and then to succinyl CoA. In normal cells with competent mitochondrial function, succinyl CoA may serve as an energy source by entering tricarboxylic acid (TCA) cycle and generating ATP through mitochondrial oxidative phosphorylation. In this case, the intracellular generation of propionic acid may protect the normal cells by providing an alternative energy source for the cells with competent mitochondrial function. This protective effect may not be available to cancer cells with mitochondrial respiration defect or under hypoxic conditions, since succinyl CoA is not an effective energy source without mitochondrial respiration. As such, Glycolycin provides a novel biochemical mechanism to preferentially kill cancer cells with respiration injury, which is prevalent in a wide spectrum of human cancers, and thus improves therapeutic selectivity, in some embodiments.

Also, the compositions of the present invention are more effective in therapeutic activity (as demonstrated in representative in vitro studies provided herein) than other glycolytic inhibitors comprising a similar mechanism of action. In particular, Glycolycin is more stable, more effective in depleting cellular ATP, and exhibits greater in vitro anticancer activity (10-20 fold more potent as measured by IC₅₀) than currently available glycolytic inhibitors with a similar mechanism of action. Furthermore, in vivo studies in animals (mice) suggest that this compound is well tolerated. No obvious toxicity was observed in mice at tested doses (S-Glycolycin 5 mg/kg, i.v., three times per week, M/W/F, or Glycolycin 6 mg/kg, i.p. daily for three days, or E-Glycolycin, 5 mg/kg, i.v. daily for 5 days), for example.

The Glycolycin compositions of the invention may be administered to a cell in any manner. In particular embodiments, the composition may be comprised in a pharmaceutically acceptable diluent. In further aspects of the invention, the composition is comprised in or with a carrier. The carrier may be any kind suitable to facilitate delivery of the composition to its intended destination, although in particular embodiments the carrier is a slow-release carrier. Specific examples of carriers useful in the invention include liposomes, nanoparticles, or biodegradable polymers.

In one embodiment of the present invention, there is a composition comprising the following general formula:

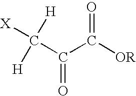

wherein X is a halogen and the composition is further characterized as follows: (a) wherein R is a covalently bonded alkyl group comprising from one or more carbon atoms, such as three or more carbon atoms, although any number of carbons may be suitable; or (b) wherein R is a metal ion, and wherein the composition further comprises a stabilizing agent. In a specific embodiment, the halogen is a bromine. In another specific the alkyl group is an aliphatic group, such as, for example, a methyl group, an ethyl group, a propyl group, a butanol group, or a pentanol group, a hexanol group, a heptanol group, or an octanol group. In another specific embodiment, the alkyl group is a ring structure, such as one comprising a cycloalkanol, a benzene derivative, a steroid group with a side chain, or a steroid group without a side chain.

Wherein the composition comprises a metal ion, any suitable metal ion may be employed, although in a specific embodiment the metal ion is further defined as an alkali metal ion, such as, for example, sodium.

In a specific embodiment, the stabilizing agent comprises carbonic acid. Furthermore, the composition of (a), noted above, may be further defined as sodium 3-halo-2-oxopropionate, and the composition may comprise hydrogen bonding between sodium 3-halo-2-oxopropionate and carbonic acid. Furthermore, the composition of (a) is further defined as sodium 3-halo-2-oxopropionate and the sodium 3-halo-2-oxopropionate and the stabilizing agent are present in a desired ratio, such as about 2:1 of sodium 3-halo-2-oxopropionate to stabilizing agent, respectively.

In a specific embodiment, the compositions described herein further comprise one or more alternate energy agents, such as, for example, a metabolic intermediate, including, for example, a metabolic intermediate in the tricarboxylic acid (TCA) cycle, is a metabolic intermediate in mitochondrial respiration, is a metabolic intermediate in both the TCA cycle and mitochondrial respiration, is a precursor to the TCA cycle, is a precursor of a metabolic intermediate in mitochondrial respiration, or is a precursor of a metabolic intermediate in both the TCA cycle and mitochondrial respiration. In a specific embodiment, the metabolic intermediate comprises glutamine, pyruvate, a fatty acid, or a combination thereof. In a further specific embodiment, the fatty acid comprises an alkyl chain having no double bonds or comprises an alkyl chain having one or more double bonds.

Compositions of the present invention may be comprised in a pharmaceutical formulation.

In another embodiment of the present invention, there is a method for inhibiting glycolysis in a cell, comprising delivering to the cell a composition of the present invention. The method may be further defined as inducing apoptosis or necrosis in said cell or inhibiting proliferation in said cell, and the cell may be a cancer cell. The cancer cell may be comprised in an individual, in a solid tumor, or both. The cancer cell may be a leukemia cell, breast cancer cell, lung cancer cell, prostate cancer cell, pancreatic cancer cell, colon cancer cell, head and neck cancer cell, liver cancer cell, bone cancer cell, ovarian cancer cell, cervical cancer cell, spleen cancer cell, brain cancer cell, esophageal cancer cell, or skin cancer cell. In specific embodiments, the cancer cell is a drug-resistant cancer cell and may be, for example, a leukemia cell. In a specific embodiment, the cancer cell is in a hypoxic environment.

In an additional embodiment of the present invention, there is a method of treating cancer in an individual, comprising the step of administering to the individual a therapeutically effective amount of a composition of the present invention. The method may further comprise administering to the individual an additional cancer therapy, such as, for example, radiation, chemotherapy, surgery, gene therapy, immunotherapy, hormone therapy, or a combination thereof. The additional cancer therapy may be administered to the individual prior to the administration of the composition of the present invention, concomitant with the administration of the composition of claim 1, subsequent to the administration of the composition of claim 1, or a combination thereof. In a specific embodiment, at least some of the cancer of the individual resides in a hypoxic environment, which may be a solid tumor.

In another embodiment of the present invention, there is a kit comprising a composition of the present invention housed in a suitable container. In a specific embodiment, the composition of (a) comprises sodium 3-bromo-2-oxopropionate, and the sodium 3-bromo-2-oxopropionate and the stabilizing agent may be housed in the same container or in separate containers. In a specific embodiment, the container comprises sterilized CO₂ gas in its void volume. The kit may further comprise a pharmaceutically acceptable diluent.

In an additional embodiment of the present invention, there is a method of producing the composition (b) of claim 1, comprising the steps of: (1) providing 3-halo-2-oxoproprionate; (2) providing an aliphatic alcohol having from one to about 5 carbon atoms; and (3) providing acidic conditions and heat, wherein an ester derivative of 3-halo-2-oxoproprionate is generated, thereby producing the composition (b), as described above.

In an additional embodiment of the present invention, there is a kit for producing a composition comprising sodium 3-halo-2-oxopropionate and carbonic acid, comprising 3-halopyruvate; and sodium carbonate, sodium bicarbonate, or both. The kit may further comprise carbon dioxide gas, water, or both. In specific embodiments, the 3-halopyruvate is 3-bromopyruvate, 3-fluoropyruvate, 3-iodopyruvate, or 3-chloropyruvate.

In another embodiment, there is a method for producing a composition comprising sodium 3-bromo-2-oxopropionate and carbonic acid, the method comprising: providing 3-bromo-2-oxopropionic acid; providing sodium carbonate; and mixing the 3-bromo-2-oxopropionic acid and the sodium carbonate in an effective ratio to produce the composition. In a specific embodiment, the 3-bromo-2-oxopropionic acid and the sodium carbonate are mixed in a ratio of about 2:1 or about 3:1. In further specific embodiments, the 3-bromo-2-oxopropionic acid, the sodium carbonate, or both are comprised in a solution, which may be, for example, water. The method may be further defined as producing a solution of the composition. The method may further comprise the step of adjusting the pH of the solution is to about 7.0 and/or of filtering the solution.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1 shows the chemical formula, molecular weight, and chemical structure of Glycolycin.

FIG. 2 shows one exemplary method of Glycolycin preparation. All starting materials can be obtained from commercial sources, for example. There are 5 main steps in the preparation of Glycolycin. These procedures are explained in more detail in Example 1.

FIG. 3 illustrates the intracellular metabolism of Glycolycin and the biochemical mechanisms for therapeutic selectivity.

FIG. 4 shows an example of depletion of cellular ATP by Glycolycin and S-Glycolycin in human leukemia cells. Cellular ATP and other nucleotides were extracted from the cells at 12 h (before significant cell death occurred), and analyzed by high-pressure liquid chromatography (HPLC). Note that the depletion of ATP by Glycolycin or S-Glycolycin also led to a secondary depletion of other nucleotides in the cells.

FIG. 5 shows the inhibitory effect of Glycolycin on the growth of human leukemia cells in comparison with 3-bromopyruvate. Cell growth inhibition was measured by the MTT assay.

FIG. 6 shows the potent cytotoxic activity of Glycolycin in human leukemia (HL-60) cells. Apoptosis was measured by annexin-V/PI staining follow by flow cytometry analysis.

FIG. 7 shows the cytotoxic activity of Glycolycin in primary leukemia isolated from 2 patients with chronic lymphocytic leukemia (CLL). Cytotoxicity was measured in vitro by MTT assay (72-h incubation).

FIG. 8 shows the cell growth inhibitory effect of Glycolycin on human solid tumor cells SKOV3 (ovarian cancer, A) and U87MG (malignant brain tumor, B). Cell growth inhibition was measured in vitro by MTT assay (72-h incubation).

FIG. 9 shows exemplary methods for preparation of E-glycolycin and M-Glycolycin.

FIG. 10 shows one exemplary method of S-glycolycin preparation and one exemplary form of S-Glycolycin in aqueous solution. Four molecules of sodium 3-bromo-2-oxopropionate and two molecules of carbonic acid are illustrated, held together by multiple hydrogen bonds.

FIG. 11 illustrates solution of S-Glycolycin and 3-bromopyruvate sodium salt without carbonic acid (3-BrPA) at various times after preparation (pH 7.0, stored at 4° C.).

FIG. 12 shows the comparison of the cell growth inhibitory effect of Glycolycin, E-Glycolycin, M-Glycolycin, S-Glycolycin, and the parental compound 3-bromopyruvate in human leukemia HL-60 cells (MTT assay, 72-h).

FIG. 13 illustrates the induction of apoptosis by S-Glycolycin, E-Glycolycin, and M-Glycolycin in HL-60 cells. Apoptosis was detected by double staining with annexin-V and PI followed by flow cytometry analysis.

FIG. 14 shows dephosphorylation of the pro-apoptotic protein BAD by incubation with S-Glycolycin, E-Glycolycin, and M-Glycolycin in human cancer cells.

FIG. 15 demonstrates depletion of cellular ATP pool by S-Glycolycin and 3-bromopyruvate (3-BrPA). Note that S-glycolycin is much more potent than 3-BrPA.

FIG. 16 shows induction of apoptosis by S-Glycolycin and 3-bromopyruvate (3-BrPA). HL-60 cells were incubated with the indicated concentrations of Glycolycin or 3-BrPA for 24 h. Apoptosis was measured by double staining with annexin-V and PI, followed by flow cytometry analysis.

FIG. 17 shows the effect of S-Glycolycin and 3-bromopyruvate on the expression of pro-apoptotic factor BAD and its phosphorylation status (Ser112).

FIG. 18 shows a time- and dose-dependent effect of S-Glycolycin on the expression level of pro-apoptotic factor BAD and its phosphorylation at Ser112.

FIG. 19 shows inhibition of glycolysis leads to more effective killing of cancer cells with respiration defect.

FIG. 20 shows HPLC analysis of cellular ATP (A), depletion of cellular ATP by 3-BrPA (B), and induction of BAD dephosphorylation by 3-BrPA and S-Glycolycin (C) in primary leukemia cells isolated from patients with chronic lymphocytic leukemia (CLL).

FIG. 21 demonstrates cells that are resistant to multi drugs (doxorubicin or adriamycin, and vinscritine) remain sensitive to inhibition of glycolysis and depletion of cellular ATP by 3-BrPA.

FIG. 22 shows that inhibition of glycolysis by 3-bromopyruvate can significantly increase the cytotoxic activity of ara-C or doxorubicin in multi drug resistant (HL-60/AR) cells (annexin-V/PI assay, 24 h).

FIG. 23 shows the growth inhibition of parental HL-60 cell line and its multi drug-resistant clone HL-60/AR incubated with the indicated concentrations of doxorubicin, vincristine, ara-C, and S-Glycolycin for 72 h. S-Glycolycin inhibited both the parental cells and multi drug resistant cells with same potency.

FIG. 24 shows a biochemical mechanism to protect normal cells by appropriate combination of S-Glycolycin and certain metabolic intermediates.

FIG. 25 demonstrates that glutamine partially protects the respiration-competent cells from the cytotoxic effect of S-Glycolycin.

FIG. 26 shows that glutamine does not protect respiration-deficient cells from the cytotoxic effect of S-Glycolycin.

FIG. 27 shows the effect of S-Glycolycin on human colon cancer HCT16 cells and malignant brain tumor U87MG cells.

FIG. 28 demonstrates that overexpression of Bcl-2 protein in human leukemia HL-60 cells by transfection with Bcl-2 did not protect the cells from the cytotoxic effect of S-Glycolycin (A), but renders the cells less sensitive to ara-C(C), and doxorubicin (D). Cell growth inhibition was measured by MTT assay.

FIG. 29 demonstrates that human lymphoma cells (Raji) are significantly more sensitive to S-Glycolycin (40 μM) under hypoxic conditions than under normoxic conditions, whereas the cellular sensitivity to doxorubicin (0.3 μM) is slightly reduced under hypoxic conditions. (Annexin-V/PI assay, 24 h)

FIG. 30 demonstrates that human colon cancer (HCT116) cells are significantly more sensitive to glycolytic inhibition by 3-BrPA under hypoxic conditions than under normoxic conditions, whereas the cellular sensitivity to doxorubicin is reduced under hypoxic conditions (Annexin-V/PI assay).

FIG. 31 demonstrates human colon cancer cells (HTC116) are more resistant to radiation treatment (6 Gy) under hypoxic conditions and under normoxic condition. However, the colon cancer cells are more sensitive to S-Glycolycin under hypoxic conditions. Combination of S-Glycolycin (40 μM) and radiation (6 Gy) effectively kills the colon cancer cells under hypoxic conditions (reduced colony formation is more than 99.9%). These results suggest that combination of Glycolycin and radiation may be an extremely effective treatment for solid tumors under hypoxic conditions.

FIG. 32 provides the chemical structure of P-glycolycin.

FIG. 33 shows methods for preparation of P-glycolycin.

FIG. 34 demonstrates the effect of Glycolycin and P-glycolycin on tumor growth in nude mice. Animals (5 mice/group) were inoculated with human ovarian cancer cells (SKOV3) by s.c. inoculation. Glycolycin and P-glycolycin were administered by i.v. injection using the indicated dose-schedules, starting on day 11 after tumor inoculation.

FIGS. 35A-35D show synergistic cytotoxic effect of rapamycin and glycolycin (3-BrOP) in human lymphoma and leukemia cells. In FIG. 35A, the human lymphoma Raji cells were treated with 3-BrOP (30 μM), rapamycin (100 ng/ml), or their combinations as indicated, and apoptosis was measured by flow cytometry analysis after the cells were double-stained with annexin-V and PI as described in the Examples. In case of drug combination, cells were first treated with rapamycin for 18 h before addition of 3-BrOP and incubated for an additional 24 h or 48 h. In FIG. 35B, there is concentration-dependent induction of loss of cell viability by 3-BrOP in the presence and absence of rapamycin (100 ng/ml, 24 h) in Raji cells. Cells were double-stained with annexin-V and PI followed by flow cytometry analysis, and viable cells were expressed as % of control. Solid bars, cells treated with 3-BrOP alone; dotted bars, cells treated with 3-BrOP plus rapamycin. The data represent the means and standard deviations of three independent experiments. (*) indicates a statistical significant difference between samples treated with 3-BrOP alone and the samples incubated with 3-BrOP and rapamycin (p<0.05). In FIG. 35C, there is concentration-dependent induction of apoptosis by 3-BrOP in the presence and absence of rapamycin (100 ng/ml, 24 h) in HL-60 cells. Cell viability was determined as described above. In FIG. 35D, a combination index of rapamycin and 3-BrOP in HL-60 and Raji cells were calculated using the Median Dose-Effect program by by Chou and Talalay18, using all data points where a single agent alone did not cause more than 90% cell killing.

FIG. 36 shows growth inhibition by rapamycin and 3-BrOP in Raji cells. Cells in exponentially growing phase were treated with 100 ng/ml rapamycin at time 0, and 3-BrOP (30 μM) was added 18 h later. Cell culture was continued for up to 72 h, and cell numbers in the samples were directly counted at the indicated time intervals, using a Coulter Z2 Particle Counter & Size Analyzer to determine the total particle numbers.

FIGS. 37A-37B show effect of rapamycin and ara-C on apoptotic response in Raji cells. (a) Raji cells were treated with rapamycin (100 ng/ml, 48 h) and ara-C (0.5 and 1 μM, 30 h), alone or in combination as indicated. In case of drug combination, cells were first treated with rapamycin for 18 h before addition of ara-C and incubation for an additional 30 h. Apoptosis were analyzed by annexin/PI assay as described in the Examples. Representative flow cytometry analyses are illustrated in (FIG. 37A), and quantitative data are shown in (FIG. 37B). Solid bars, cells treated with ara-C alone; dotted bars, cells treated with ara-C plus rapamycin. There was no significant statistical difference between % apoptosis in cells treated with ara-C alone and the combination ara-C and rapamycin.

FIG. 38 demonstrates that combination of glycolycin (3-BrOP) and rapamycin caused severe ATP depletion in Raji cells. Cells were first treated with rapamycin (100 ng/ml) for 18 h, and then incubated with the indicated concentrations of 3-BrOP for another 6 h. Cellular ATP was measured by HPLC analysis as described in Materials and Methods, and expressed as % of the control. The ATP content of the control cells was 2.2±0.2 nmol/10⁶ cells. Results were expressed as the mean±SD of three independent experiments. The symbol * indicates a significant statistical difference (p<0.05).

FIG. 39 provides a synergistic inhibition of cellular glucose uptake by rapamycin and glycolycin (3-BrOP). Cells were first treated with rapamycin (100 ng/ml) for 18 h, and then incubated with the indicated concentrations of 3-BrOP for an additional 2 h. The samples were washed with fresh warm medium, and 5×10⁶ cells were re-suspended in 5 ml RPMI 1640 media (glucose-free) containing 0.2 μCi/ml [³H] 2-deoxyglucose and incubated for 60 min. The cellular uptake of radioactive 2-deoxyglucose was determined by liquid scintillation counting as described in Examples. The drug effect on glucose uptake is expressed as % of the control cells. The radioactive glucose uptake in the control sample was 5740±633 CPM/10⁶ cells. Results are mean±SD of three independent experiments. The symbol * indicates a significant statistical difference (p<0.05).

FIG. 40 shows the effect of rapamycin and glycolycin (3-BrOP) on phosphorylation of the mTOR downstream targets. Raji cells were treated with Rapamycin (100 ng/ml) for 24 h, and/or the indicated concentrations of 3-BrOP for 6 hours. In case of drug combination, cells were first incubated with rapamycin for 18 h, and then with 3-BrOP for an additional 6 h. Equal amounts of cellular protein extracts were resolved by SDS-PAGE, and blotted for p-p70S6K(Thr389), p-p70S6K(Thr421/Ser424), p-4E-BP-1 (Ser65), p-BAD (Ser112), and total BAD protein, using respective antibodies. Beta-Actin was also blotted as protein loading control.

### DETAILED DESCRIPTION OF THE INVENTION

I. Definitions

As used herein the specification, “a” or “an” may mean one or more. As used herein in the claim(s), when used in conjunction with the word “comprising”, the words “a” or “an” may mean one or more than one. As used herein “another” may mean at least a second or more. Some embodiments of the invention may consist of or consist essentially of one or more elements, method steps, and/or methods of the invention. It is contemplated that any method or composition described herein can be implemented with respect to any other method or composition described herein.

The term “aliphatic” as used herein refers to a group comprising no carbon-carbon double or triple bonds.

The term “alkyl” as used herein refers to a carbon-based group, including those having saturated carbon-carbon bonds or those having one or more unsaturated carbon-carbon bonds.

The term “alternate energy agent” as used herein refers to a composition that directly or indirectly may be utilized as a substrate or precursor for a pathway that generates energy, such as in the form of ATP. The precursor may be an immediate precursor for entry into the pathway or it may be further upstream from an immediate precursor for entry into the pathway.

The term “Glycolycin composition” as used herein refers to either Glycolycin, a derivative thereof, or Glycolycin or a derivative thereof in combination with another component to facilitate or enhance delivery and/or action of Glycolycin. The additional component may be utilized to protect cells unintended for Glycolycin action. In particular embodiments, the additional component is an alternate energy agent, such as a metabolic intermediate, for example one that is an intermediate in energy source pathway in a cell. Examples of such pathways include TCA cycle, mitochondrial respiration, and so forth.

The term “Glycolycin derivate” as used herein refers to a chemical composition derived from 3-halo-2-oxopropionate by the esterification methods similar to the procedures described herein for the preparation of Glycolycin, where the 3-halogen group may be a fluorine, a chlorine, a bromine, or an iodine, and the alcohol used for esterification may contains three or more carbons. A glycolycin derivative will functionally inhibit glycolysis and disturb cellular energy metabolism.

The term “metabolic intermediate” as used herein refers to a composition that directly or indirectly is utilized in an energy-producing pathway in a cell, such as the TCA cycle and/or mitochondrial respiration. The metabolic intermediate may be a substrate for the energy-producing pathway, or it may be a precursor thereto, either an immediate precursor or a precursor further upstream from the pathway.

II. The Present Invention

The present invention exploits the metabolic difference between cancer and normal cells by utilizing a biochemical basis for developing novel compounds and therapeutic strategies that specifically target the glycolytic pathway, thereby preferentially depleting the energy supply in and selectively killing cancer cells. The invention employs compositions and methods related to novel anticancer agents, Glycolycin and its derivatives, which is able to effectively block the glycolytic pathway by targeting a key enzyme in this metabolic pathway. As described herein, in vitro studies demonstrated that Glycolycin causes a severe depletion of the cellular ATP pool and massive cell death in cancer cells, especially in cells with mitochondrial defect due to mutations in the mitochondrial DNA, or in cancer cells under hypoxic conditions that render them resistant to conventional anticancer agents and radiation. Inhibition of glycolysis by this compound also induces apoptosis mediated by dephosphorylation of BAD, a molecule involved in the integration of apoptosis and glycolysis. Studies of Glycolycin in animals, such as mice, demonstrate no apparent toxicity at the dosage of 5 mg/kg.

Furthermore, compared to a currently available compound with a similar mechanism of action, Glycolycin has superior pharmaceutical properties, including greater stability, less molecular polarity for better cellular permeability, simple synthesis procedures, and cost-effectiveness for scale-up pharmaceutical production. Importantly, Glycolycin exhibits a significantly greater anticancer activity in vitro than other compounds with a similar mechanism of action. Since the Warburg effect is commonly seen in a wide spectrum of human cancers, Glycolycin is effective against a variety of cancer types, and thus has broad therapeutic applications. Furthermore, because hypoxia is commonly present in most solid tumors and renders the cancer cells less sensitive to many anticancer agents and radiation therapy, the remarkable activity of Glycolycin to kill cancer cells under hypoxic conditions and/or to enhance the activity of other agents and radiation indicates that Glycolycin can be used to effectively treat solid tumor in a hypoxic environment in vivo. A mechanism-based drug combination is also provided herein pursuant to an additional embodiment of the present invention wherein a Glycolycin composition comprising one or more additional ingredients that improve their therapeutic selectivity, thereby substantially protecting non-cancerous cells. In additional aspects of the invention, the glycolycin composition further comprises or is delivered in combination with radiation and/or a drug. Any suitable drug may be employed, although in specific embodiments the drug is an inhibitor of the mTOR pathway, such as rapamycin or other similar compounds.

III. Pharmaceutical Preparations

Pharmaceutical compositions of the present invention comprise an effective amount of one or more forms of the inventive composition, and in some embodiments an additional agent, dissolved or dispersed in a pharmaceutically acceptable carrier or excipient. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises at least one Glycolycin composition and/or a derivative thereof, and in some embodiments an additional active ingredient, will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The composition may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, rectally, topically, intratumorally, intramuscularly, intraperitoneally, subcutaneously, intravesicularlly, mucosally, intrapericardially, orally, topically, locally, using aerosol, injection, infusion, continuous infusion, localized perfusion bathing target cells directly, via a catheter, via a lavage, in cremes, mouthwashes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal, such as a patient, can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 2 milligram/kg/body weight, about 3 milligram/kg/body weight, about 4 milligram/kg/body weight, about 5 milligram/kg/body weight, about 10 milligram/kg/body weight, about 30 milligram/kg/body weight, about 50 milligram/kg/body weight, and about 100 milligram/kg/body weight or more per administration, and any range derivable therein. In non-limiting examples of a derivable range from the numbers listed herein, a range of about 1 mg/kg/body weight to about 10 mg/kg/body weight, about 5 microgram/kg/body weight to about 100 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifingal agents, including but not limited to parabens (e.g., methylparabens, propylparabens), chlorobutanol, phenol, sorbic acid, thimerosal or combinations thereof.

The composition may be formulated into a composition in a free ester form with or without 1-propanol or other alcohol, or in neutral or salt form. Pharmaceutically acceptable salts, include the acid addition salts, e.g., those formed with inorganic acids such as for example, hydrochloric or sulfuric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, mouthwashes, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments, a glycolycin composition is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or mannitol. The preparation of highly concentrated compositions for direct injection is also contemplated, where the use of 1-propanol as solvent is envisioned to result in rapid penetration, delivering high concentrations of the active agents to a small area.

The composition must be stable under the conditions of manufacture and storage, and preserved against the contaminating action of microorganisms, such as bacteria and fungi.

In particular embodiments, prolonged absorption of an injectable composition can be brought about by the use in the compositions of agents delaying absorption, such as, for example, aluminum monostearate, gelatin or combinations thereof.

IV. Therapeutic Applications

The compositions and methods of the present invention are particularly suitable for use in one or more therapeutic applications. In general, the application for Glycolycin and derivative compositions is one in which it is therapeutic for glycolysis to be inhibited or at least reduced. Any application for which glycolysis inhibition is therapeutic is suitable for the invention, although in particular embodiments the therapeutic application encompasses damage or eradication of at least one cancer cell. In particular embodiments, the cancer cell resides in a tissue or group of cells comprising a hypoxic environment, such as a solid tumor. In particular, the cancer cell is subjected to apoptosis or necrosis as a direct or indirect result of the invention. In specific embodiments, the cancer cell is in an animal, such as in a mammal, for example a human. In some embodiments, proliferation of the cancer cell is at least reduced following treatment with at least one Glycolycin composition. Thus, a preferred embodiment of the invention comprises cancer treatment as its therapeutic application.

The cancer to be treated may be any kind of cancer, but in particular embodiments the cancer is colon cancer, lung cancer, breast cancer, prostate cancer, pancreatic cancer, ovarian cancer, liver cancer, bone cancer, head and neck cancer, leukemia, lymphoma, brain cancer, melanoma, spleen cancer, cervical cancer, kidney cancer, throat cancer, malignant glioma, bladder cancer, sarcoma, or mesotheliomas, for example.

Although the Glycolycin composition may be administered for a therapeutic application in any manner that provides a therapeutically effective amount of the composition, in particular embodiments the composition is administered intravenously, intradermally, intraarterially, intraperitoneally, intralesionally, intracranially, intraarticularly, intraprostaticaly, intrapleurally, intratracheally, intranasally, intravitreally, intravaginally, rectally, topically, intratumorally, intramuscularly, intraperitoneally, subcutaneously, intravesicularlly, mucosally, intrapericardially, orally, topically, locally, using aerosol, injection, infusion, continuous infusion, localized perfusion bathing target cells directly, via a catheter, via a lavage, in cremes, mouthwashes, in lipid compositions (e.g., liposomes), or by other method or any combination of the forgoing as would be known to one of ordinary skill in the art. The composition is therapeutically effective if at least one treated cancer cell is damaged or eradicated. In some embodiments, at least one symptom of the medical condition being treated is ameliorated.

The Glycolycin composition for the therapeutic application may comprise an additional agent to enhance its effectiveness, such as an additional agent to enhance the ability of the composition to target particular cells, to protect desirable cells, to increase the activity of the composition, provide greater stability to the composition, and/or augment the delivery of the composition, for example. In particular embodiments of the present invention, agents are utilized that protect cells from treatment with Glycolycin, such as protecting non-cancerous cells in an application comprising cancer treatment. These agents may be of any kind suitable for the purpose, although in particular embodiments the agents are alternative energy agents that provide normal cells with an alternative energy source other than glycolysis. For example, the alternative energy agent may be a compound that facilitates, enhances, or increases the use by the cell of energy-generating pathways other than glycolysis, such as the TCA cycle, mitochondrial respiration, or both. These compounds may be administered in conjunction with Glycolycin, or they may derive from processing of Glycolycin itself upon entry into the individual being treated, such as upon entry into a cell of the individual being treated.

In specific aspects of the invention, the alternative energy source is a metabolic intermediate and/or precursor to an energy-generating pathway that is not glycolysis. That is, the metabolic intermediate may be a bona fide intermediate of the energy-generating pathway or it may be a precursor to the energy-generating pathway. In the event that the intermediate is in fact a precursor to one or more energy-generating pathways, it may be an immediate precursor, or it may be one that is not an immediate precursor to the pathway, such as one being further upstream of the pathway. In particular embodiments of the invention, providing appropriate or even copious amounts of the one or more intermediates increases the event of entering into such alternative energy-generating pathways. Specifically, glutamine, pyruvate, or fatty acids are suitable metabolic intermediates in the invention. Thus, the Glycolycin compositions comprising metabolic intermediates, such as Glycolycin-P or Glycolycin-G, are particularly suited for therapeutic applications in this manner, such as the exemplary application of cancer treatment.

In specific aspects of the invention, Glycolycin is particularly applicable to treatment of cancers that grow in one or more hypoxic tissue environments. The lack or insufficient oxygen in the hypoxic tumor tissue renders the cancer cells highly dependent on glycolysis for their ATP generation, and most vulnerable to glycolytic inhibition. The hypoxia condition in solid tumors also leads to the development of drug resistance and reduced sensitivity to radiation therapy. The potent inhibitory effect of Glycolycin on glycolysis is most useful in treatment of cancer cells in a hypoxic environment. The present inventors have demonstrated that Glycolycin, either alone or in combination with radiation, for example, is very effective in killing cancer cells culture under hypoxic conditions.

V. Hypoxia

Tissue hypoxia occurs where there is an imbalance between oxygen supply and consumption. Hypoxia occurs in solid tumors as a result of an inadequate supply of oxygen, due to exponential cellular proliferation and an inefficient vascular supply. Tumor hypoxia is a major constraint for cancer therapy, and particularly radiotherapy and many types of chemotherapy, and this is associated with unfavorable prognosis, regardless of the treatment modality applied. Hypoxia is related to malignant progression, increased invasion, angiogenesis and an increased risk of metastasis formation. In specific embodiments, hypoxia is furthermore a stressor that selects cells with increased resistance to apoptosis and thereby indirectly contributes to treatment resistance. Hypoxia may be characterized by the following aspects: 1) there is direct interference of hypoxia with antineoplastic treatment modalities; that is, the efficacy of ionizing radiation and a variety of cytotoxic drugs and cytokines relies directly on adequate oxygen tensions; and 2) there are notable effects of hypoxia on the biology of tumor and stromal cells.

The expression of several genes controlling tumor cell survival are regulated by hypoxia, e.g., growth factors governing the formation of new blood vessels, and hypoxia-responsive transcription factors modulating the expression of genes, which promote tumor cell survival. There are a variety of pathways by which tumor hypoxia leads to chemotherapeutic resistance, such as directly due to lack of oxygen availability, and indirectly due to alterations in the proteome/genome, angiogenesis and pH changes.

The present invention provides particularly well-suited novel compositions and methods to combat the difficulties that hypoxic conditions provide for cancer therapy, given that tumor metabolism can encompass hypoxic-related molecular processes.

VI. Combination Treatments

In order to increase the effectiveness of a Glycolycin composition, it may be desirable to combine a Glycolycin composition of the present invention with one or more other agents effective in the treatment of hyperproliferative disease, such as anti-cancer agents. An “anti-cancer” agent is a chemical or physical modality (e.g. radiation) capable of negatively affecting cancer in a subject, for example, by killing cancer cells, inducing apoptosis and/or necrosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the agent(s) or multiple factor(s) at the same time or sequentially. This may be achieved by contacting the cell with a single Glycolycin composition or pharmacological formulation that includes both agents, or by contacting the cell with two distinct compositions or formulations, at the same time or sequentially, wherein one composition includes the Glycolycin composition and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with another therapy, such as gene therapy. For example, the herpes simplex-thymidine kinase (HS-tK) gene, when delivered to brain tumors by a retroviral vector system, successfully induced susceptibility to the antiviral agent ganciclovir (Culver et al., 1992). In the context of the present invention, it is contemplated that a glycolycin composition could be used similarly in conjunction with chemotherapeutic, radiotherapeutic, surgical, or immunotherapeutic intervention, for example, in addition to other pro-apoptotic or cell cycle regulating agents. The ability of Glycolycin to enhance the activity of other anticancer agents such as doxorubicin, ara-C, and radiation has been demonstrated in vitro by the current inventors.

Alternatively, the additional or supplemental therapy, such as gene therapy, may precede or follow the other agent treatment by intervals ranging from minutes to weeks. In embodiments where the Glycolycin composition and the other agent and expression construct are applied separately to the cell, one would generally ensure that a significant period of time did not expire between the time of each delivery, such that the Glycolycin composition and the other agent would still be able to exert an advantageously combined effect on the cell. In such instances, it is contemplated that one may contact the cell with both modalities within about 12-24 h of each other and, more preferably, within about 2-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, such as wherein the Glycolycin composition is “A” and the secondary agent, such as radio- or chemotherapy, is “B”, for example:

A/B/A B/A/B B/B/A A/A/B A/B/B B/A/A A/B/B/B B/A/B/B

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the therapeutic compositions of the present invention to a patient will follow general protocols for the administration of chemotherapeutics. It is expected that the treatment cycles would be repeated as necessary. It also is contemplated that various standard therapies, as well as surgical intervention, may be applied in combination with the described cell therapy with Glycolycin.

A. Chemotherapy

A skilled artisan recognizes that in addition to the Glycolycin compositions encompassed by the invention for the purpose of inhibiting cell growth or killing cancer cells, other chemotherapeutic agents are useful in the treatment of neoplastic disease. Examples of such chemotherapeutic agents that can be used in conjunction with Glycolycin are described in the following Table 1.

TABLE 1
Chemotherapeutic Agents Useful In Neoplastic Disease

| | | NONPROPRIETARY | |
|---|---|---|---|
| | TYPE OF | NAMES | |
| CLASS | AGENT | (OTHER NAMES) | DISEASE |
| Alkylating Agents | Nitrogen Mustards | Mechlorethamine | Hodgkin's disease, |
| | | (HN₂) | non-Hodgkin's lymphomas |
| | | Cyclophosphamide | Acute and chronic |
| | | Ifosfamide | lymphocytic leukemias, |
| | | | Hodgkin's disease, |
| | | | non-Hodgkin's |
| | | | lymphomas, multiple |
| | | | myeloma, |
| | | | neuroblastoma, breast, |
| | | | ovary, lung, Wilms' |
| | | | tumor, cervix, testis, |
| | | | soft-tissue sarcomas |
| | | Melphalan | Multiple myeloma, breast, |
| | | (L-sarcolysin) | ovary |
| | | Chlorambucil | Chronic lymphocytic |
| | | | leukemia, primary |
| | | | macroglobulinemia, |
| | | | Hodgkin's disease, |
| | | | non-Hodgkin's lymphomas |
| | Ethylenimenes and | Hexamethylmelamine | Ovary |
| | Methylmelamines | Thiotepa | Bladder, breast, ovary |
| | Alkyl Sulfonates | Busulfan | Chronic granulocytic |
| | | | leukemia |
| | Nitrosoureas | Carmustine (BCNU) | Hodgkin's disease, |
| | | | non-Hodgkin's lymphomas, |
| | | | primary brain tumors, |
| | | | multiple myeloma, malignant |
| | | | melanoma |
| | | Lomustine (CCNU) | Hodgkin's disease, |
| | | | non-Hodgkin's lymphomas, |
| | | | primary brain tumors, |
| | | | small-cell lung |
| | | Semustine | Primary brain tumors, |
| | | (methyl-CCNU) | stomach, colon |
| | | Streptozocin | Malignant pancreatic |
| | | (streptozotocin) | insulinoma, malignant |
| | | | carcinoid |
| | Triazines | Dacarbazine (DTIC; | Malignant melanoma, |
| | | dimethyltriazenoimidazolecarboxamide) | Hodgkin's disease, soft-tissue |
| | | | sarcomas |
| Antimetabolites | Folic Acid | Methotrexate | Acute lymphocytic |
| | Analogs | (amethopterin) | leukemia, choriocarcinoma, |
| | | | mycosis fungoides, breast, |
| | | | head and neck, lung, |
| | | | osteogenic sarcoma |
| | Pyrimidine | Fluouracil | Breast, colon, stomach, |
| | Analogs | (5-fluorouracil; 5-FU) | pancreas, ovary, head and |
| | | Floxuridine | neck, urinary bladder, |
| | | (fluorode-oxyuridine; | premalignant skin lesions |
| | | FUdR) | (topical) |
| | | Cytarabine (cytosine | Acute granulocytic and |
| | | arabinoside) | acute lymphocytic leukemias |
| | Purine Analogs | Mercaptopurine | Acute lymphocytic, acute |
| | and Related | (6-mercaptopurine; | granulocytic and chronic |
| | Inhibitors | 6-MP) | granulocytic leukemias |
| | | Thioguanine | Acute granulocytic, acute |
| | | (6-thioguanine; TG) | lymphocytic and chronic |
| | | | granulocytic leukemias |
| | | Pentostatin | Hairy cell leukemia, mycosis |
| | | (2-deoxycoformycin) | fungoides, chronic |
| | | | lymphocytic leukemia |
| Natural Products | Vinca Alkaloids | Vinblastine (VLB) | Hodgkin's disease, |
| | | | non-Hodgkin's lymphomas, |
| | | | breast, testis |
| | | Vincristine | Acute lymphocytic |
| | | | leukemia, neuroblastoma, |
| | | | Wilms' tumor, |
| | | | rhabdomyosarcoma, |
| | | | Hodgkin's disease, |
| | | | non-Hodgkin's lymphomas, |
| | | | small-cell lung |
| | Epipodophyllotoxins | Etoposide | Testis, small-cell lung and |
| | | Tertiposide | other lung, breast, Hodgkin's |
| | | | disease, non-Hodgkin's |
| | | | lymphomas, acute |
| | | | granulocytic leukemia, |
| | | | Kaposi's sarcoma |
| | Antibiotics | Dactinomycin | Choriocarcinoma, Wilms' |
| | | (actinomycin D) | tumor, rhabdomyosarcoma, |
| | | | testis, Kaposi's sarcoma |
| | | Daunorubicin | Acute granulocytic and |
| | | (daunomycin; | acute lymphocytic leukemias |
| | | rubidomycin) | |
| | | Doxorubicin | Soft-tissue, osteogenic and |
| | | | other sarcomas; Hodgkin's |
| | | | disease, non-Hodgkin's |
| | | | lymphomas, acute leukemias, |
| | | | breast, genitourinary, |
| | | | thyroid, lung, stomach, |
| | | | neuroblastoma |
| | | Bleomycin | Testis, head and neck, skin, |
| | | | esophagus, lung and |
| | | | genitourinary tract; |
| | | | Hodgkin's disease, |
| | | | non-Hodgkin's lymphomas |
| | | Plicamycin | Testis, malignant |
| | | (mithramycin) | hypercalcemia |
| | | Mitomycin (mitomycin | Stomach, cervix, colon, |
| | | C) | breast, pancreas, bladder, |
| | | | head and neck |
| | Enzymes | L-Asparaginase | Acute lymphocytic leukemia |
| | Biological | Interferon alfa | Hairy cell leukemia., |
| | Response | | Kaposi's sarcoma, |
| | Modifiers | | melanoma, carcinoid, renal |
| | | | cell, ovary, bladder, |
| | | | non-Hodgkin's lymphomas, |
| | | | mycosis fungoides, multiple |
| | | | myeloma, chronic |
| | | | granulocytic leukemia |
| Miscellaneous | Platinum | Cisplatin (cis-DDP) | Testis, ovary, bladder, head |
| Agents | Coordination | Carboplatin | and neck, lung, thyroid, |
| | Complexes | | cervix, endometrium, |
| | | | neuroblastoma, osteogenic |
| | | | sarcoma |
| | Anthracenedione | Mitoxantrone | Acute granulocytic |
| | | | leukemia, breast |
| | Substituted Urea | Hydroxyurea | Chronic granulocytic |
| | | | leukemia, polycythemia vera, |
| | | | essental thrombocytosis, |
| | | | malignant melanoma |
| | Methyl Hydrazine | Procarbazine | Hodgkin's disease |
| | Derivative | (N-methylhydrazine, | |
| | | MIH) | |
| | Adrenocortical | Mitotane (o,p′-DDD) | Adrenal cortex |
| | Suppressant | Aminoglutethimide | Breast |
| Hormones and | Adrenocorti | Prednisone (several | Acute and chronic |
| Antagonists | costeroids | other equivalent | lymphocytic leukemias, |
| | | preparations available) | non-Hodgkin's lymphomas, |
| | | | Hodgkin's disease, breast |
| | Progestins | Hydroxyprogesterone | Endometrium, breast |
| | | caproate | |
| | | Medroxyprogesterone | |
| | | acetate | |
| | | Megestrol acetate | |
| | Estrogens | Diethylstilbestrol | Breast, prostate |
| | | Ethinyl estradiol (other | |
| | | preparations available) | |
| | Antiestrogen | Tamoxifen | Breast |
| | Androgens | Testosterone | Breast |
| | | propionate | |
| | | Fluoxymesterone (other | |
| | | preparations available) | |
| | Antiandrogen | Flutamide | Prostate |
| | Gonadotropin- | Leuprolide | Prostate |
| | releasing hormone analog | | |

In addition to the chemotherapeutic agents listed above, any analog or derivative variant of the those listed may be used in conjunction with Glycolycin and are within the scope of the invention.

B. Radiotherapy

In addition to the Glycolycin compositions described herein for the purpose of inhibiting cell growth, radation-based therapies are useful. That is, other factors that cause DNA damage and have been used extensively include what are commonly known as γ-rays, X-rays, high energy proton or electron beams and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells. The repair of DNA damage requires sufficient energy supply in form of ATP. In one embodiment of the present invention, it is contemplated that inhibition of glycolysis and depletion of ATP by Glycolycin can severely suppress the ability of cancer cells to repair the DNA damage by radiation, and thus significantly enhance the therapeutic activity.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with Glycolycin therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

D. Genes

In yet another embodiment, the secondary treatment is a gene therapy in which a therapeutic polynucleotide is administered before, after, or at the same time as a Glycolycin composition of the present invention. Delivery of the inventive composition with at least one vector encoding one of the following exemplary gene products will have a combined anti-hyperproliferative effect on target tissues. A variety of proteins are encompassed within the invention, some of which are described below.

1. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

2. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue.

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G1. The activity of this enzyme may be to phosphorylate Rb at late GI. The activity of CDK4 is controlled by an activating subunit, D-type cyclin, and by an inhibitory subunit, the p161NK4 has been biochemically characterized as a protein that specifically binds to and inhibits CDK4, and thus may regulate Rb phosphorylation (Serrano et al., 1993; Serrano et al., 1995). Since the p161NK4 protein is a CDK4 inhibitor (Serrano, 1993), deletion of this gene may increase the activity of CDK4, resulting in hyperphosphorylation of the Rb protein. p16 also is known to regulate the function of CDK6.

p161NK4 belongs to a newly described class of CDK-inhibitory proteins that also includes p16B, p19, p21Waf1/Cip1, and p27KIP1. The p161NK4 gene maps to a chromosome region frequently deleted in many tumor types. Homozygous deletions and mutations of the p161NK4 gene are frequent in human tumor cell lines. This evidence suggests that the p161NK4 gene is a tumor suppressor gene. This interpretation has been challenged, however, by the observation that the frequency of the p161NK4 gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p161NK4 function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, Bik/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., HIF-1, VEGF, FGF, thrombospondin, BAI-1, GDAIF, or their receptors) and MCC.

3. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., BclXL, BclW, BclS, Mcl-1, A1, Bfl-1) or counteract Bcl-2 function and promote cell death (e.g., Bax, Bak, Bik, Bim, Bid, Bad, Harakiri).

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the Glycolycin treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers, for example. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

VII. Lipid Compositions

In certain embodiments, the present invention concerns a novel composition comprising one or more lipids associated with at least one Glycolycin composition. A lipid is a substance that is characteristically insoluble in water and extractable with an organic solvent. Lipids include, for example, the substances comprising the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes. Of course, compounds other than those specifically described herein that are understood by one of skill in the art as lipids are also encompassed by the compositions and methods of the present invention.

A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof.

A. Lipid Types

A neutral fat may comprise a glycerol and a fatty acid. A typical glycerol is a three carbon alcohol. A fatty acid generally is a molecule comprising a carbon chain with an acidic moeity (e.g., carboxylic acid) at an end of the chain. The carbon chain may of a fatty acid may be of any length, however, it is preferred that the length of the carbon chain be of from about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, to about 30 or more carbon atoms, and any range derivable therein. However, a preferred range is from about 14 to about 24 carbon atoms in the chain portion of the fatty acid, with about 16 to about 18 carbon atoms being particularly preferred in certain embodiments. In certain embodiments the fatty acid carbon chain may comprise an odd number of carbon atoms, however, an even number of carbon atoms in the chain may be preferred in certain embodiments. A fatty acid comprising only single bonds in its carbon chain is called saturated, while a fatty acid comprising at least one double bond in its chain is called unsaturated.

Specific fatty acids include, but are not limited to, linoleic acid, oleic acid, palmitic acid, linolenic acid, stearic acid, lauric acid, myristic acid, arachidic acid, palmitoleic acid, arachidonic acid ricinoleic acid, tuberculosteric acid, lactobacillic acid. An acidic group of one or more fatty acids is covalently bonded to one or more hydroxyl groups of a glycerol. Thus, a monoglyceride comprises a glycerol and one fatty acid, a diglyceride comprises a glycerol and two fatty acids, and a triglyceride comprises a glycerol and three fatty acids.

A phospholipid generally comprises either glycerol or a sphingosine moiety, an ionic phosphate group to produce an amphipathic compound, and one or more fatty acids. Types of phospholipids include, for example, phophoglycerides, wherein a phosphate group is linked to the first carbon of glycerol of a diglyceride, and sphingophospholipids (e.g., sphingomyelin), wherein a phosphate group is esterified to a sphingosine amino alcohol. Another example of a sphingophospholipid is a sulfatide, which comprises an ionic sulfate group that makes the molecule amphipathic. A phopholipid may, of course, comprise further chemical groups, such as for example, an alcohol attached to the phosphate group. Examples of such alcohol groups include serine, ethanolamine, choline, glycerol and inositol. Thus, specific phosphoglycerides include a phosphatidyl serine, a phosphatidyl ethanolamine, a phosphatidyl choline, a phosphatidyl glycerol or a phosphotidyl inositol. Other phospholipids include a phosphatidic acid or a diacetyl phosphate. In one aspect, a phosphatidylcholine comprises a dioleoylphosphatidylcholine (a.k.a. cardiolipin), an egg phosphatidylcholine, a dipalmitoyl phosphalidycholine, a monomyristoyl phosphatidylcholine, a monopalmitoyl phosphatidylcholine, a monostearoyl phosphatidylcholine, a monooleoyl phosphatidylcholine, a dibutroyl phosphatidylcholine, a divaleroyl phosphatidylcholine, a dicaproyl phosphatidylcholine, a diheptanoyl phosphatidylcholine, a dicapryloyl phosphatidylcholine or a distearoyl phosphatidylcholine.

A glycolipid is related to a sphinogophospholipid, but comprises a carbohydrate group rather than a phosphate group attached to a primary hydroxyl group of the sphingosine. A type of glycolipid called a cerebroside comprises one sugar group (e.g., a glucose or galactose) attached to the primary hydroxyl group. Another example of a glycolipid is a ganglioside (e.g., a monosialoganglioside, a GM1), which comprises about 2, about 3, about 4, about 5, about 6, to about 7 or so sugar groups, that may be in a branched chain, attached to the primary hydroxyl group. In other embodiments, the glycolipid is a ceramide (e.g., lactosylceramide).

A steroid is a four-membered ring system derivative of a phenanthrene. Steroids often possess regulatory functions in cells, tissues and organisms, and include, for example, hormones and related compounds in the progestagen (e.g., progesterone), glucocoricoid (e.g., cortisol), mineralocorticoid (e.g., aldosterone), androgen (e.g., testosterone) and estrogen (e.g., estrone) families. Cholesterol is another example of a steroid, and generally serves structural rather than regulatory functions. Vitamin D is another example of a sterol, and is involved in calcium absorption from the intestine.

A terpene is a lipid comprising one or more five carbon isoprene groups. Terpenes have various biological functions, and include, for example, vitamin A, coenyzme Q and carotenoids (e.g., lycopene and β-carotene).

B. Charged and Neutral Lipid Compositions

In certain embodiments, a lipid component of a composition is uncharged or primarily uncharged. In one embodiment, a lipid component of a composition comprises one or more neutral lipids. In another aspect, a lipid component of a composition may be substantially free of anionic and cationic lipids, such as certain phospholipids (e.g., phosphatidyl choline) and cholesterol. In certain aspects, a lipid component of an uncharged or primarily uncharged lipid composition comprises about 95%, about 96%, about 97%, about 98%, about 99% or 100% lipids without a charge, substantially uncharged lipid(s), and/or a lipid mixture with equal numbers of positive and negative charges.

In other aspects, a lipid composition may be charged. For example, charged phospholipids may be used for preparing a lipid composition according to the present invention and can carry a net positive charge or a net negative charge. In a non-limiting example, diacetyl phosphate can be employed to confer a negative charge on the lipid composition, and stearylamine can be used to confer a positive charge on the lipid composition.

C. Making Lipids

Lipids can be obtained from natural sources, commercial sources or chemically synthesized, as would be known to one of ordinary skill in the art. For example, phospholipids can be from natural sources, such as egg or soybean phosphatidylcholine, brain phosphatidic acid, brain or plant phosphatidylinositol, heart cardiolipin and plant or bacterial phosphatidylethanolamine. In another example, lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). In certain embodiments, stock solutions of lipids in chloroform or chloroform/methanol can be stored at about −20 C. Preferably, chloroform is used as the only solvent since it is more readily evaporated than methanol.

D. Lipid Composition Structures

In a preferred embodiment of the invention, the Glycolycin composition may be associated with a lipid. A Glycolycin composition associated with a lipid may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure. A lipid or lipid/Glycolycin composition of the present invention is not limited to any particular structure. For example, they may also simply be interspersed in a solution, possibly forming aggregates which are not uniform in either size or shape. In another example, they may be present in a bilayer structure, as micelles, or with a “collapsed” structure.

In certain embodiments, a lipid composition may comprise about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or any range derivable therein, of a particular lipid, lipid type or non-lipid component such as a drug, protein, sugar, nucleic acids or other material disclosed herein or as would be known to one of skill in the art. In a non-limiting example, a lipid composition may comprise about 10% to about 20% neutral lipids, and about 33% to about 34% of a cerebroside, and about 1% cholesterol. In another non-limiting example, a liposome may comprise about 4% to about 12% terpenes, wherein about 1% of the micelle is specifically lycopene, leaving about 3% to about 11% of the liposome as comprising other terpenes; and about 10% to about 35% phosphatidyl choline, and about 1% of a drug. Thus, it is contemplated that lipid compositions of the present invention may comprise any of the lipids, lipid types or other components in any combination or percentage range.

1. Emulsions

A lipid may be comprised in an emulsion. A lipid emulsion is a substantially permanent heterogenous liquid mixture of two or more liquids that do not normally dissolve in each other, by mechanical agitation or by small amounts of additional substances known as emulsifiers. Methods for preparing lipid emulsions and adding additional components are well known in the art (e.g., Modem Pharmaceutics, 1990, incorporated herein by reference).

For example, one or more lipids are added to ethanol or chloroform or any other suitable organic solvent and agitated by hand or mechanical techniques. The solvent is then evaporated from the mixture leaving a dried glaze of lipid. The lipids are resuspended in aqueous media, such as phosphate buffered saline, resulting in an emulsion. To achieve a more homogeneous size distribution of the emulsified lipids, the mixture may be sonicated using conventional sonication techniques, further emulsified using microfluidization (using, for example, a Microfluidizer, Newton, Mass.), and/or extruded under high pressure (such as, for example, 600 psi) using an Extruder Device (Lipex Biomembranes, Vancouver, Canada).

2. Micelles

A lipid may be comprised in a micelle. A micelle is a cluster or aggregate of lipid compounds, generally in the form of a lipid monolayer, and may be prepared using any micelle producing protocol known to those of skill in the art (e.g., Canfield et al., 1990; El-Gorab et al, 1973; Colloidal Surfactant, 1963; and Catalysis in Micellar and Macromolecular Systems, 1975, each incorporated herein by reference). For example, one or more lipids are typically made into a suspension in an organic solvent, the solvent is evaporated, the lipid is resuspended in an aqueous medium, sonicated and then centrifuged.

E. Liposomes

In particular embodiments, a lipid comprises a liposome. A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition.

A multilamellar liposome has multiple lipid layers separated by aqueous medium. They form spontaneously when lipids comprising phospholipids are suspended in an excess of aqueous solution. The lipid components undergo self-rearrangement before the formation of closed structures and entrap water and dissolved solutes between the lipid bilayers (Ghosh and Bachhawat, 1991). Lipophilic molecules or molecules with lipophilic regions may also dissolve in or associate with the lipid bilayer.

In certain less preferred embodiments, phospholipids from natural sources, such as egg or soybean phosphatidylcholine, brain phosphatidic acid, brain or plant phosphatidylinositol, heart cardiolipin and plant or bacterial phosphatidylethanolamine are preferably not used as the primary phosphatide, i.e., constituting 50% or more of the total phosphatide composition or a liposome, because of the instability and leakiness of the resulting liposomes.

In particular embodiments, a lipid and/or Glycolycin composition may be, for example, encapsulated in the aqueous interior of a liposome, interspersed within the lipid bilayer of a liposome, attached to a liposome via a linking molecule that is associated with both the liposome and the Glycolycin composition, entrapped in a liposome, complexed with a liposome, etc.

3. Making Liposomes

A liposome used according to the present invention can be made by different methods, as would be known to one of ordinary skill in the art. Phospholipids can form a variety of structures other than liposomes when dispersed in water, depending on the molar ratio of lipid to water. At low ratios the liposome is the preferred structure.

For example, a phospholipid (Avanti Polar Lipids, Alabaster, Ala.), such as for example the neutral phospholipid dioleoylphosphatidylcholine (DOPC), is dissolved in tert-butanol. The lipid(s) is then mixed with the Glycolycin composition, and/or other component(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline. The average diameter of the particles obtained using Tween 20 for encapsulating the Glycolycin composition is about 0.7 to about 1.0 μm in diameter.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In other alternative methods, liposomes can be prepared in accordance with other known laboratory procedures (e.g., see Bangham et al., 1965; Gregoriadis, 1979; Deamer and Uster 1983, Szoka and Papahadjopoulos, 1978, each incorporated herein by reference in relevant part). These methods differ in their respective abilities to entrap aqueous material and their respective aqueous space-to-lipid ratios.

The dried lipids or lyophilized liposomes prepared as described above may be dehydrated and reconstituted in a solution of inhibitory peptide and diluted to an appropriate concentration with an suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000×g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

The size of a liposome varies depending on the method of synthesis. Liposomes in the present invention can be a variety of sizes. In certain embodiments, the liposomes are small, e.g., less than about 200 nm, about 100 nm, about 90 nm, about 80 nm, about 70 nm, about 60 nm, or less than about 50 nm in external diameter. In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040; U.K. Patent Application GB 2193095 A; Mayer et al., 1986; Hope et al., 1985; Mayhew et al. 1987; Mayhew et al., 1984; Cheng et al., 1987; and Liposome Technology, 1984, each incorporated herein by reference).

A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

The production of lipid formulations often is accomplished by sonication or serial extrusion of liposomal mixtures after (I) reverse phase evaporation (II) dehydration-rehydration (III) detergent dialysis and (IV) thin film hydration. In one aspect, a contemplated method for preparing liposomes in certain embodiments is heating sonicating, and sequential extrusion of the lipids through filters or membranes of decreasing pore size, thereby resulting in the formation of small, stable liposome structures. This preparation produces liposomal/° or liposomes only of appropriate and uniform size, which are structurally stable and produce maximal activity. Such techniques are well-known to those of skill in the art (see, for example Martin, 1990).

Once manufactured, lipid structures can be used to encapsulate compounds that are toxic (e.g., chemotherapeutics) or labile (e.g., nucleic acids) when in circulation. The physical characteristics of liposomes depend on pH, ionic strength and/or the presence of divalent cations. Liposomes can show low permeability to ionic and/or polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and/or results in an increase in permeability to ions, sugars and/or drugs. Liposomal encapsulation has resulted in a lower toxicity and a longer serum half-life for such compounds (Gabizon et al., 1990).

Liposomes interact with cells to deliver agents via four different mechanisms: Endocytosis by phagocytic cells of the reticuloendothelial system such as macrophages and/or neutrophils; adsorption to the cell surface, either by nonspecific weak hydrophobic and/or electrostatic forces, and/or by specific interactions with cell-surface components; fusion with the plasma cell membrane by insertion of the lipid bilayer of the liposome into the plasma membrane, with simultaneous release of liposomal contents into the cytoplasm; and/or by transfer of liposomal lipids to cellular and/or subcellular membranes, and/or vice versa, without any association of the liposome contents. Varying the liposome formulation can alter which mechanism is operative, although more than one may operate at the same time.

Numerous disease treatments are using lipid based gene transfer strategies to enhance conventional or establish novel therapies, in particular therapies for treating hyperproliferative diseases. Advances in liposome formulations have improved the efficiency of gene transfer in vivo (Templeton et al., 1997) and it is contemplated that liposomes are prepared by these methods. Alternate methods of preparing lipid-based formulations for nucleic acid delivery are described (WO 99/18933).

In another liposome formulation, an amphipathic vehicle called a solvent dilution microcarrier (SDMC) enables integration of particular molecules into the bi-layer of the lipid vehicle (U.S. Pat. No. 5,879,703). The SDMCs can be used to deliver lipopolysaccharides, polypeptides, nucleic acids and the like. Of course, any other methods of liposome preparation can be used by the skilled artisan to obtain a desired liposome formulation in the present invention.

4. Liposome Targeting

Association of the Glycolycin composition with a liposome may improve biodistribution and other properties of the Glycolycin composition. For example, liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980). Successful liposome-mediated gene transfer in rats after intravenous injection has also been accomplished (Nicolau et al., 1987).

It is contemplated that a liposome/Glycolycin composition composition may comprise additional materials for delivery to a tissue. For example, in certain embodiments of the invention, the lipid or liposome may be associated with a hemagglutinating virus (HVJ). This has been shown to facilitate fusion with the cell membrane and promote cell entry of liposome-encapsulated DNA (Kaneda et al., 1989). In another example, the lipid or liposome may be complexed or employed in conjunction with nuclear non-histone chromosomal proteins (HMG-1) (Kato et al., 1991). In yet further embodiments, the lipid may be complexed or employed in conjunction with both HVJ and HMG-1.

Targeted delivery is achieved by the addition of ligands without compromising the ability of these liposomes deliver large amounts of Glycolycin composition. It is contemplated that this will enable delivery to specific cells, tissues and organs. The targeting specificity of the ligand-based delivery systems are based on the distribution of the ligand receptors on different cell types. The targeting ligand may either be non-covalently or covalently associated with the lipid complex, and can be conjugated to the liposomes by a variety of methods.

a. Cross-linkers

Bifunctional cross-linking reagents have been extensively used for a variety of purposes including preparation of affinity matrices, modification and stabilization of diverse structures, identification of ligand and receptor binding sites, and structural studies. Homobifunctional reagents that carry two identical functional groups proved to be highly efficient in inducing cross-linking between identical and different macromolecules or subunits of a macromolecule, and linking of polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

Exemplary methods for cross-linking ligands to liposomes are described in U.S. Pat. No. 5,603,872 and U.S. Pat. No. 5,401,511, each specifically incorporated herein by reference in its entirety). Various ligands can be covalently bound to liposomal surfaces through the cross-linking of amine residues. Liposomes, in particular, multilamellar vesicles (MLV) or unilamellar vesicles such as microemulsified liposomes (MEL) and large unilamellar liposomes (LUVET), each containing phosphatidylethanolamine (PE), have been prepared by established procedures. The inclusion of PE in the liposome provides an active functional residue, a primary amine, on the liposomal surface for cross-linking purposes. Ligands such as epidermal growth factor (EGF) have been successfully linked with PE-liposomes. Ligands are bound covalently to discrete sites on the liposome surfaces. The number and surface density of these sites will be dictated by the liposome formulation and the liposome type. The liposomal surfaces may also have sites for non-covalent association. To form covalent conjugates of ligands and liposomes, cross-linking reagents have been studied for effectiveness and biocompatibility. Cross-linking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl) carbodiimide (EDC). Through the complex chemistry of cross-linking, linkage of the amine residues of the recognizing substance and liposomes is established.

In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling in one example, of aldehydes to free thiols. The cross-linking reagent can be modified to cross-link various functional groups and is thus useful for cross-linking polypeptides and sugars.

In instances where a particular polypeptide does not contain a residue amenable for a given cross-linking reagent in its native sequence, conservative genetic or synthetic amino acid changes in the primary sequence can be utilized.

b. Targeting Ligands

The targeting ligand can be either anchored in the hydrophobic portion of the complex or attached to reactive terminal groups of the hydrophilic portion of the complex. The targeting ligand can be attached to the liposome via a linkage to a reactive group, e.g., on the distal end of the hydrophilic polymer. Preferred reactive groups include amino groups, carboxylic groups, hydrazide groups, and thiol groups. The coupling of the targeting ligand to the hydrophilic polymer can be performed by standard methods of organic chemistry that are known to those skilled in the art. In certain embodiments, the total concentration of the targeting ligand can be from about 0.01 to about 10% mol.

Targeting ligands are any ligand specific for a characteristic component of the targeted region. Preferred targeting ligands include proteins such as polyclonal or monoclonal antibodies, antibody fragments, or chimeric antibodies, enzymes, or hormones, or sugars such as mono-, oligo- and poly-saccharides (see, Heath et al., Chem. Phys. Lipids 40:347 (1986)) For example, disialoganglioside GD2 is a tumor antigen that has been identified neuroectodermal origin tumors, such as neuroblastoma, melanoma, small-cell lung carcenoma, glioma and certain sarcomas (Mujoo et al., 1986, Schulz et al., 1984). Liposomes containing anti-disialoganglioside GD2 monoclonal antibodies have been used to aid the targeting of the liposomes to cells expressing the tumor antigen (Montaldo et al., 1999; Pagan et al., 1999). In another non-limiting example, breast and gynecological cancer antigen specific antibodies are described in U.S. Pat. No. 5,939,277, incorporated herein by reference. In a further non-limiting example, prostate cancer specific antibodies are disclosed in U.S. Pat. No. 6,107,090, incorporated herein by reference. Thus, it is contemplated that the antibodies described herein or as would be known to one of ordinary skill in the art may be used to target specific tissues and cell types in combination with the compositions and methods of the present invention. In certain embodiments of the invention, contemplated targeting ligands interact with integrins, proteoglycans, glycoproteins, receptors or transporters. Suitable ligands include any that are specific for cells of the target organ, or for structures of the target organ exposed to the circulation as a result of local pathology, such as tumors.

In certain embodiments of the present invention, in order to enhance the transduction of cells, to increase transduction of target cells, or to limit transduction of undesired cells, antibody or cyclic peptide targeting moieties (ligands) are associated with the lipid complex. Such methods are known in the art. For example, liposomes have been described further that specifically target cells of the mammalian central nervous system (U.S. Pat. No. 5,786,214, incorporated herein by reference). The liposomes are composed essentially of N-glutarylphosphatidylethanolamine, cholesterol and oleic acid, wherein a monoclonal antibody specific for neuroglia is conjugated to the liposomes. It is contemplated that a monoclonal antibody or antibody fragment may be used to target delivery to specific cells, tissues, or organs in the animal, such as for example, brain, heart, lung, liver, etc.

Still further, a Glycolycin composition may be delivered to a target cell via receptor-mediated delivery and/or targeting vehicles comprising a lipid or liposome. These take advantage of the selective uptake of macromolecules by receptor-mediated endocytosis that will be occurring in a target cell. In view of the cell type-specific distribution of various receptors, this delivery method adds another degree of specificity to the present invention.

Thus, in certain aspects of the present invention, a ligand will be chosen to correspond to a receptor specifically expressed on the target cell population. A cell-specific Glycolycin composition delivery and/or targeting vehicle may comprise a specific binding ligand in combination with a liposome. The Glycolycin composition to be delivered are housed within a liposome and the specific binding ligand is functionally incorporated into a liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In certain embodiments, a receptor-mediated delivery and/or targeting vehicles comprise a cell receptor-specific ligand and a Glycolycin composition-binding agent. Others comprise a cell receptor-specific ligand to which Glycolycin composition to be delivered has been operatively attached. For example, several ligands have been used for receptor-mediated gene transfer (Wu and Wu, 1987; Wagner et al./1990; Perales et al., 1994; Myers, EPO 0273085), which establishes the operability of the technique. In another example, specific delivery in the context of another mammalian cell type has been described (Wu and Wu, 1993; incorporated herein by reference).

In still further embodiments, the specific binding ligand may comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). The asialoglycoprotein, asialofetuin, which contains terminal galactosyl residues, also has been demonstrated to target liposomes to the liver (Spanjer and Scherphof, 1983; Hara et al., 1996). The sugars mannosyl, fucosyl or N-acetyl glucosamine, when coupled to the backbone of a polypeptide, bind the high affinity manose receptor (U.S. Pat. No. 5,432,260, specifically incorporated herein by reference in its entirety). It is contemplated that the cell or tissue-specific transforming constructs of the present invention can be specifically delivered into a target cell or tissue in a similar manner.

In another example, lactosyl ceramide, and peptides that target the LDL receptor related proteins, such as apolipoprotein E3 (“Apo E”) have been useful in targeting liposomes to the liver (Spanjer and Scherphof, 1983; WO 98/0748).

Folate and the folate receptor have also been described as useful for cellular targeting (U.S. Pat. No. 5,871,727). In this example, the vitamin folate is coupled to the complex. The folate receptor has high affinity for its ligand and is overexpressed on the surface of several malignant cell lines, including lung, breast and brain tumors. Anti-folate such as methotrexate may also be used as targeting ligands. Transferrin mediated delivery systems target a wide range of replicating cells that express the transferrin receptor (Gilliland et al., 1980).

5. Lipid Administration

The actual dosage amount of a lipid composition (e.g., a liposome-Glycolycin composition) administered to a patient can be determined by physical and physiological factors such as body weight, severity of condition, idiopathy of the patient and on the route of administration. With these considerations in mind, the dosage of a lipid composition for a particular subject and/or course of treatment can readily be determined.

The present invention can be administered intravenously, intradermally, intraarterially, intraperitoneally, intralesionally, intracranially, intraarticularly, intraprostaticaly, intrapleurally, intratracheally, intranasally, intravitreally, intravaginally, rectally, topically, intratumorally, intramuscularly, intraperitoneally, subcutaneously, intravesicularlly, mucosally, intrapericardially, orally, topically, locally and/or using aerosol, injection, infusion, continuous infusion, localized perfusion bathing target cells directly or via a catheter and/or lavage.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention Example 1

### Glycolycin

(1) Chemical Structure

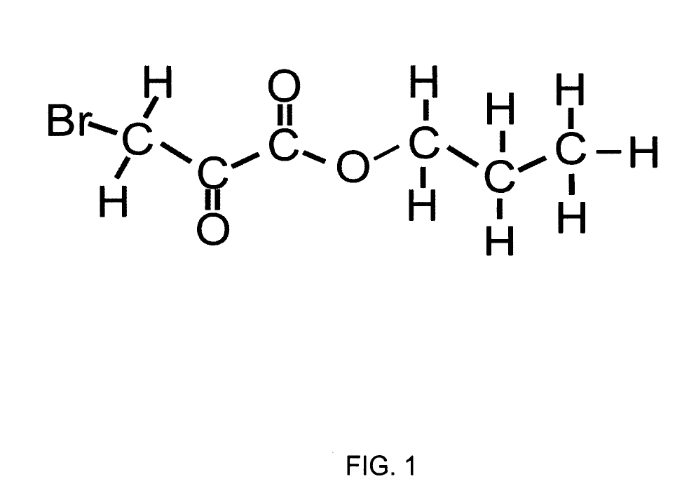

Glycolycin (propyl 3-bromo-2-oxopropionate, or 3-bromo-2-oxopropionic acid propyl ester, or propyl 3-bromopyruvate) is a small molecular weight anticancer agent. It has the chemical formula of C₆O₃H₉Br, and a molecular weight of 209. The chemical structure of Glycolycin is shown in FIG. 1.

(2) Chemical Synthesis and Preparation

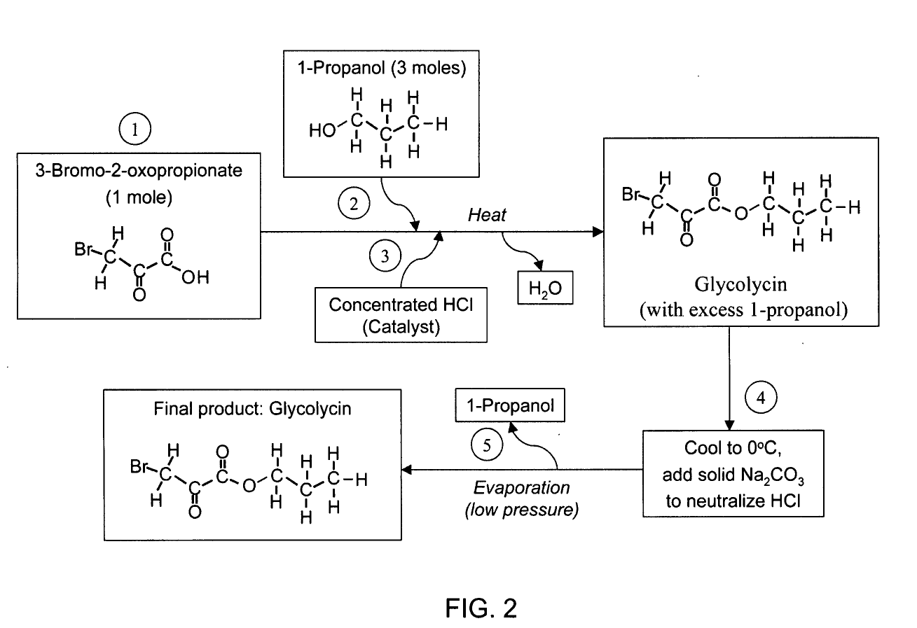

An exemplary method to prepare Glycolycin is illustrated in FIG. 2. All starting materials can be obtained from commercial sources, for example. The major components include 3-bromo-2-oxopropionate (also known as 3-bromopyruvate), 1-propanol, concentrated hydrochloric acid (HCl), and sodium carbonate (Na₂CO₃, or alternatively sodium bicarbonate, NaHCO₃). The principle chemical reaction is the esterification of 3-bromo-2-oxopropionate by 1-propanol. There are at least five specific steps in the preparation of Glycolycin: (1) Place 1 mole of 3-bromo-2-oxopropionate solid (167 grams) in a reaction chamber. (2) Add 3 moles of pure 1-propanol to the reaction chamber. The excess molar amount of 1-propanol favors the chemical reaction toward fully utilization of 3-bromo-2-oxopropionate to produce Glycolycin with favorable yield. (3) Add a small amount of concentrated hydrochloric acid as the catalyst for the esterification reaction, heat the chamber to 40° C., and let the reaction continue under constant stirring. After 60 min, cool to 30° C., and let the reaction continue for additional 8 hours with constant stirring. (4) After the reaction is completed, cool the reaction products to 0° C. with an ice-bath, add solid Na₂CO₃ (or NaHCO₃) in the amount only sufficient to neutralize the added HCl. Do not use excess Na₂CO₃ (or NaHCO₃) or add water, since excess Na₂CO₃ (or NaHCO₃) or the presence of water facilitates the hydrolysis of Glycolycin. (5) Evaporate off the excess 1-propanol under low pressure with proper vacuum but without heating. Alternatively, Glycolycin can be stored with the excess 1-propanol at −20° C. The presence of 1-propanol stabilizes Glycolycin by minimizing its hydrolysis.

(3) Metabolism and Mechanism of Drug Action

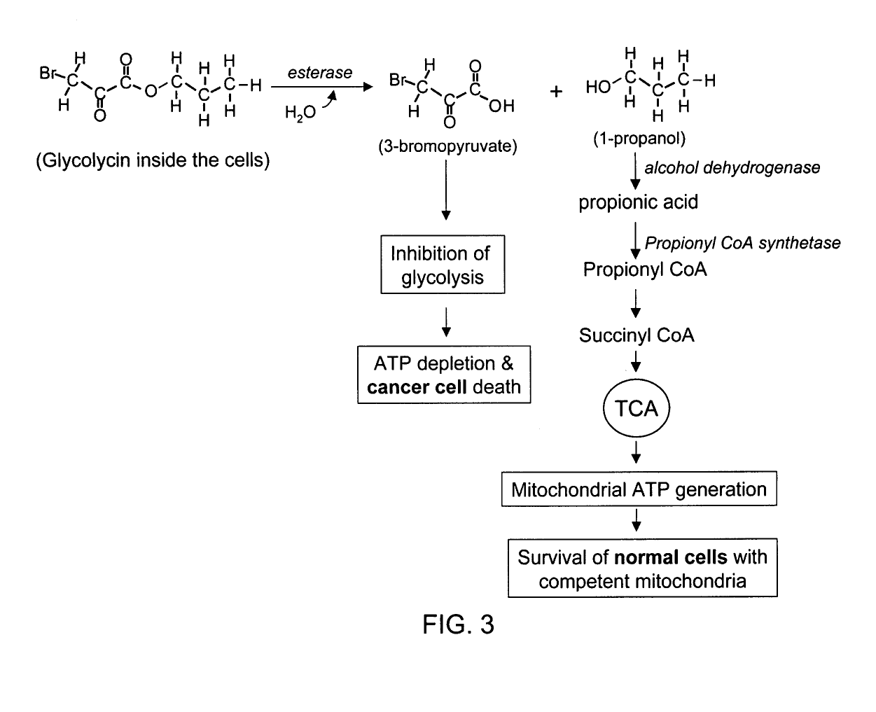

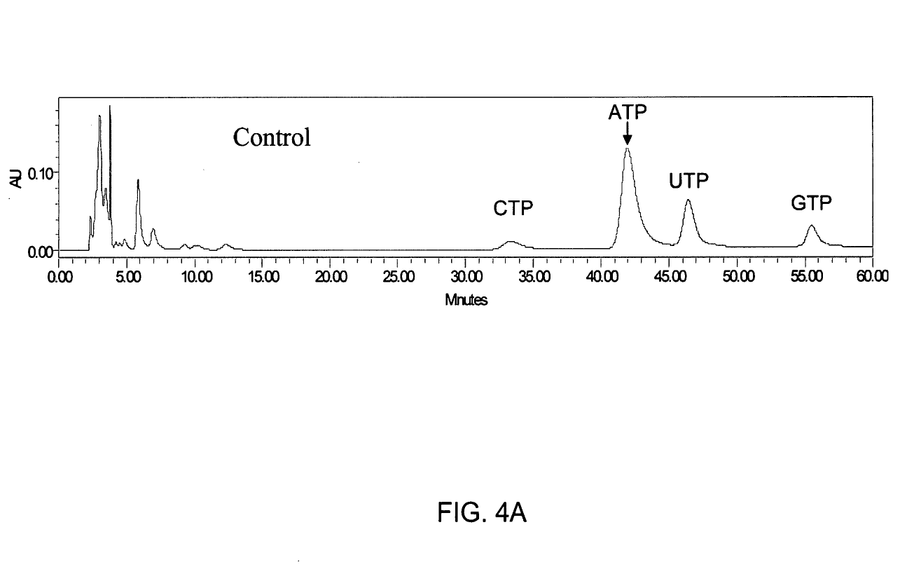

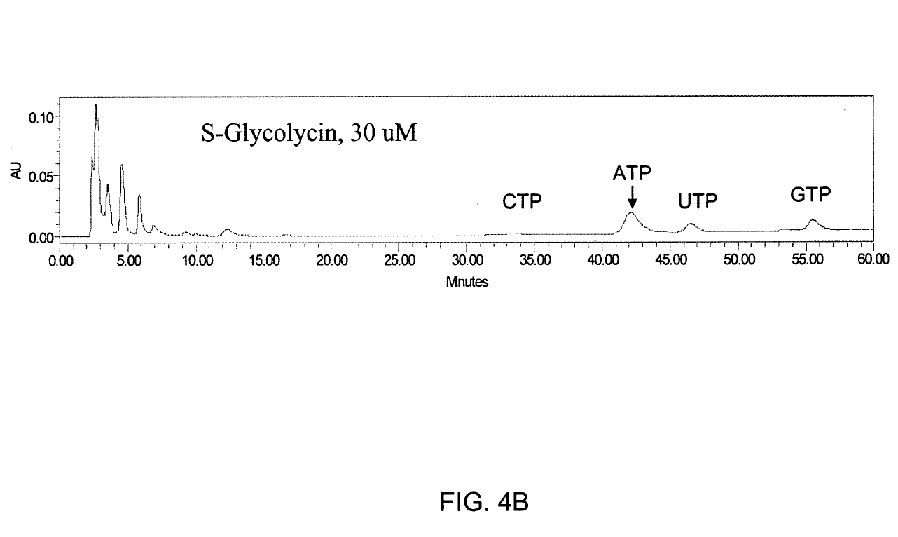

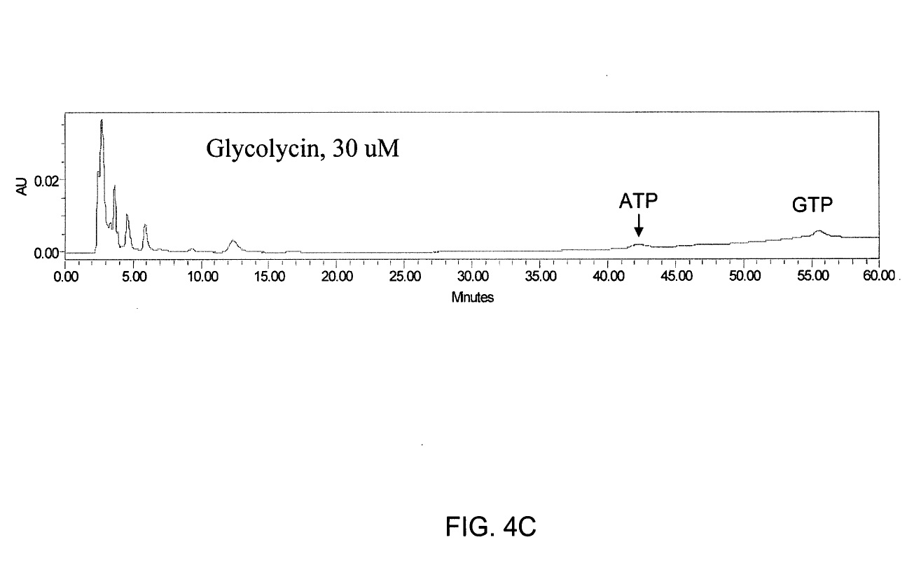

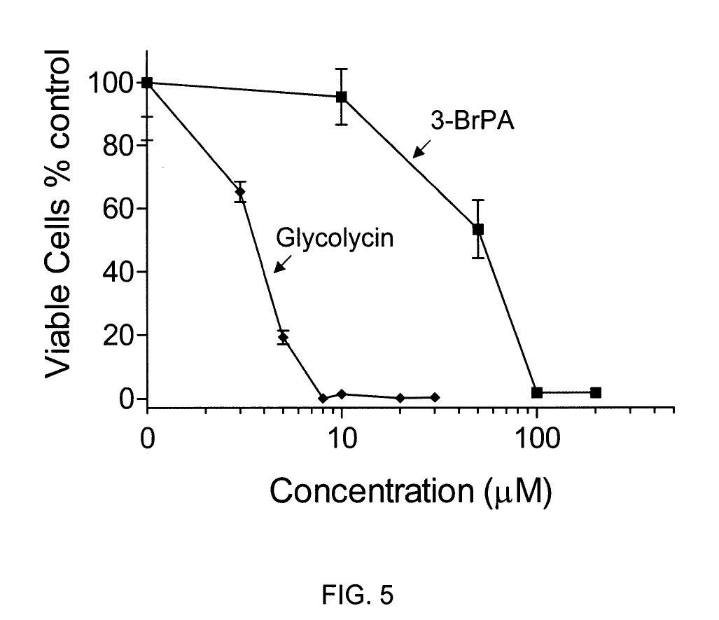

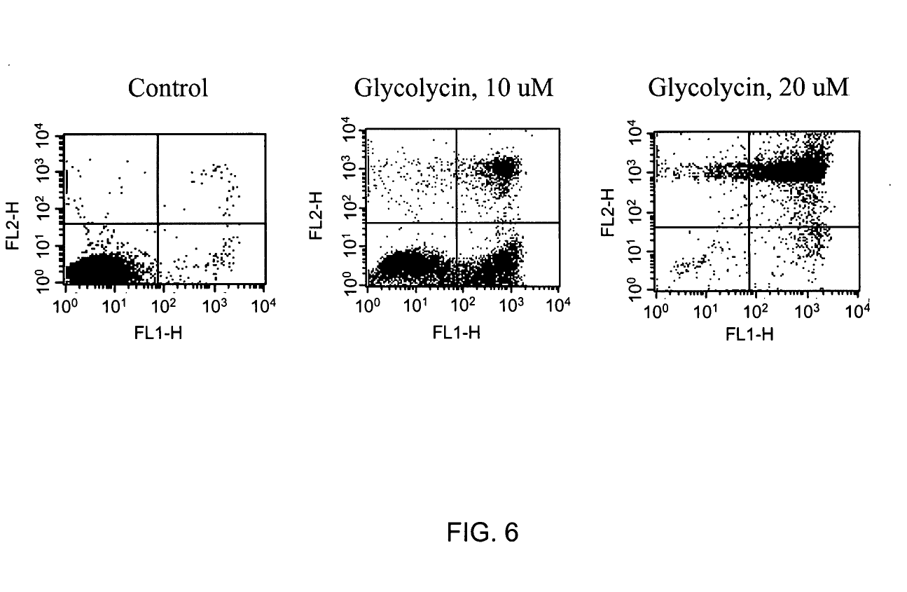

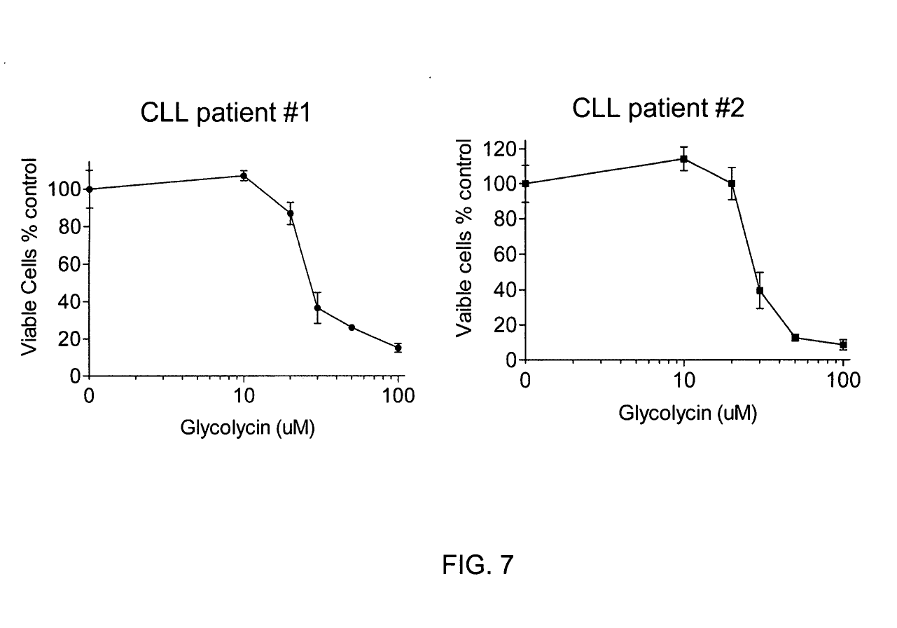

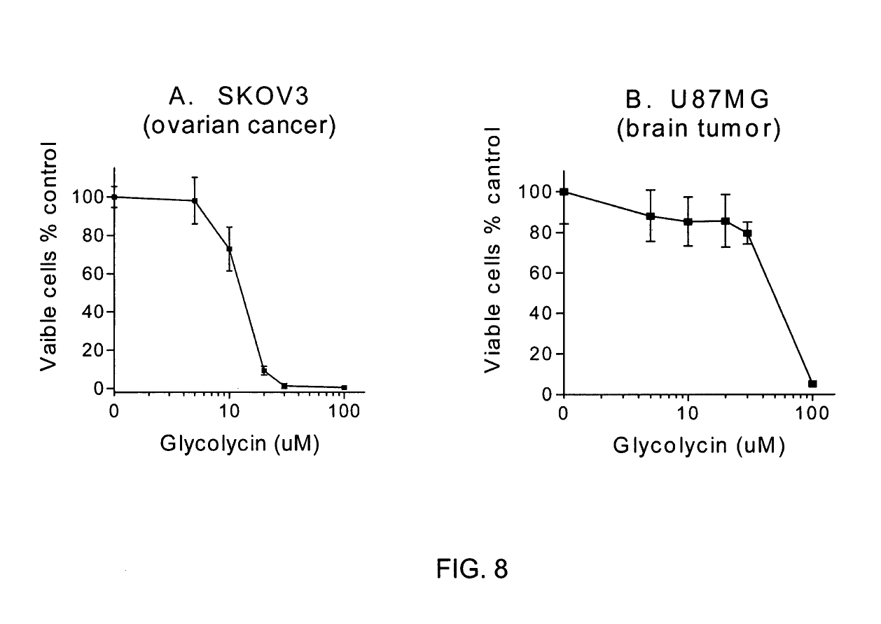

Glycolycin is an ester and chemically less polarized than 3-bromo-2-oxopropionate, and thus is readily permeable through the cellular membranes. Once inside the cells, Glycolycin is cleaved by esterase, generating the active component. 3-bromo-2-oxopropionate and a metabolic substrate 1-propanol. The accumulation of intracellular 3-bromo-2-oxopropionate effectively inhibits glycolysis, leading to severe depletion of cellular ATP, as shown in FIG. 4, and massive killing of cancer cells of various tissue origins, as illustrated in FIGS. 5-8. The other hydrolytic product, 1-propanol, can be further converted to succinyl CoA by a series of metabolic reactions in the cells. These exemplary metabolic conversion steps are illustrated in FIG. 3. It should be noted that in normal cells with competent mitochondrial function, succinyl CoA may serve as an energy source by entering the tricarboxylic acid (TCA) cycle and generating ATP through the mitochondrial oxidative phosphorylation. In this case, the intracellular generation of propionic acid may protect the normal cells to some extent by providing an alternative energy source. This protective effect may not be available to cancer cells with mitochondrial respiration defect or under hypoxic conditions, since succinyl CoA is not an effective energy source without mitochondrial respiration. Thus, the intracellular hydrolysis of Glycolycin provides a novel biochemical mechanism to preferentially kill cancer cells with respiration defect, which is prevalent in a wide spectrum of human cancers and in solid tumor under hypoxic conditions.

### Example 2

### E-Glycolycin and M-Glycolycin

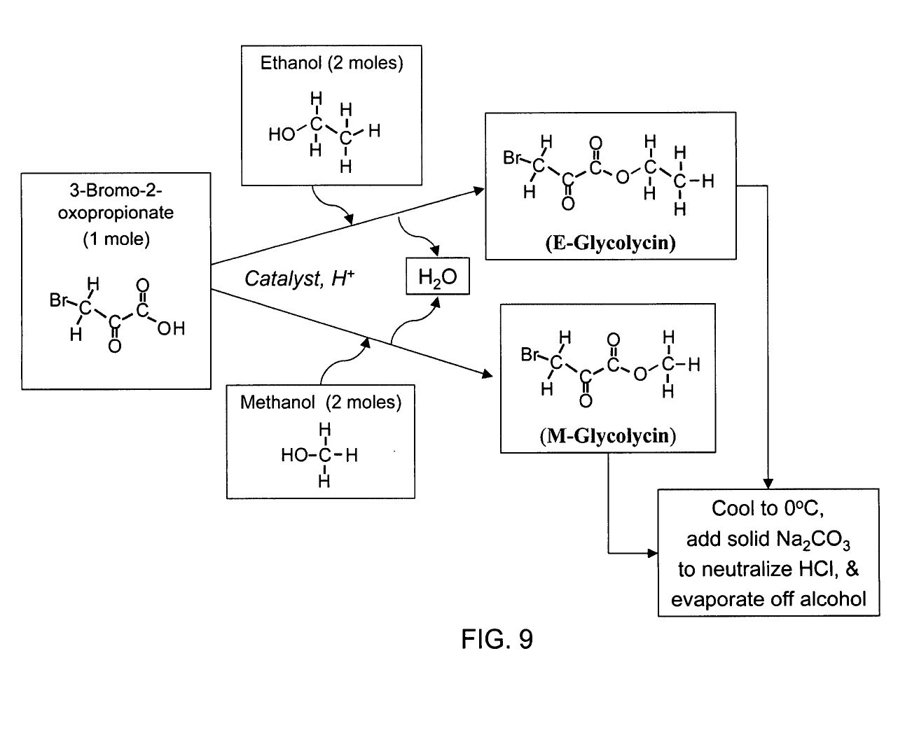

E-Glycolycin (Ethyl 3-bromo-2-oxopropionate, or 3-bromo-2-oxopropionic acid ethyl ester) has the chemical formula of C₅O₃H₇Br and a molecular weight of 195, whereas M-Glycolycin (Methyl 3-bromo-2-oxopropionate, or 3-bromo-2-oxopropionic acid methyl ester) has the chemical formula of C₄O₃H₅Br and a molecular weight of 181. The chemical structures of E-Glycolycin and M-Glycolycin are shown in FIG. 9.

FIG. 9 also illustrates exemplary methods to prepare E-Glycolycin and M-Glycolycin. All starting materials can be obtained from commercial sources, for example. The major components include 3-bromo-2-oxopropionate, ethanol, methanol, concentrated hydrochloric acid (HCl), and sodium carbonate (Na₂CO₃, or alternatively sodium bicarbonate NaHCO₃). Similar to the preparation of Glycolycin, the principal chemical reaction to produce E-Glycolycin or M-Glycolycin is also the esterification of 3-bromo-2-oxopropionate by proper alcohols. Esterification of 3-bromo-2-oxopropionate by ethanol produces E-Glycolycin, whereas esterification of 3-bromo-2-oxopropionate by methanol generates M-Glycolycin. There are at least five specific steps each in the preparation of E-Glycolycin or M-Glycolycin: (1) Place 1 mole of 3-bromo-2-oxopropionate solid (167 grams) in each reaction chamber; (2) Add 2 moles of pure ethanol (for E-Glycolycin preparation) or methanol (for M-Glycolycin preparation) to the respective reaction chamber; the excess molar amount of alcohols favors the chemical reaction toward fully utilization of 3-bromo-2-oxopropionate with favorable yield of E-Glycolycin of M-Glycolycin; (3) Add a small amount of concentrated hydrochloric acid to each chamber as the catalyst for the esterification reaction, heat the chamber to 40° C., and let the reaction continue under constant stirring. After 60 min, cool to 30° C., and let the reaction continue for additional 8 hours with constant stirring; (4) After the reaction is completed, cool the reaction products to 0° C. with an ice-bath, add solid Na₂CO₃ (or NaHCO₃) in the amount only sufficient to neutralize the added HCl. Do not use excess Na₂CO₃ (or NaHCO₃) or add water, since excess Na₂CO₃ (or NaHCO₃) or the presence of water facilitates hydrolysis of the ester products; (5) Evaporate off the excess alcohol under low pressure with proper vacuum but without heating. Alternatively, E-Glycolycin stored with the excess ethanol −20° C.

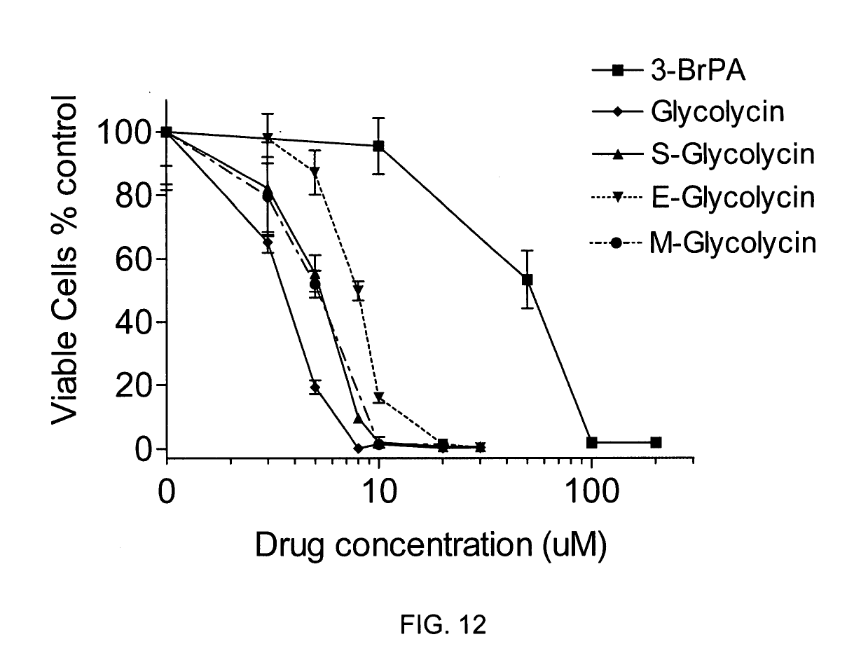

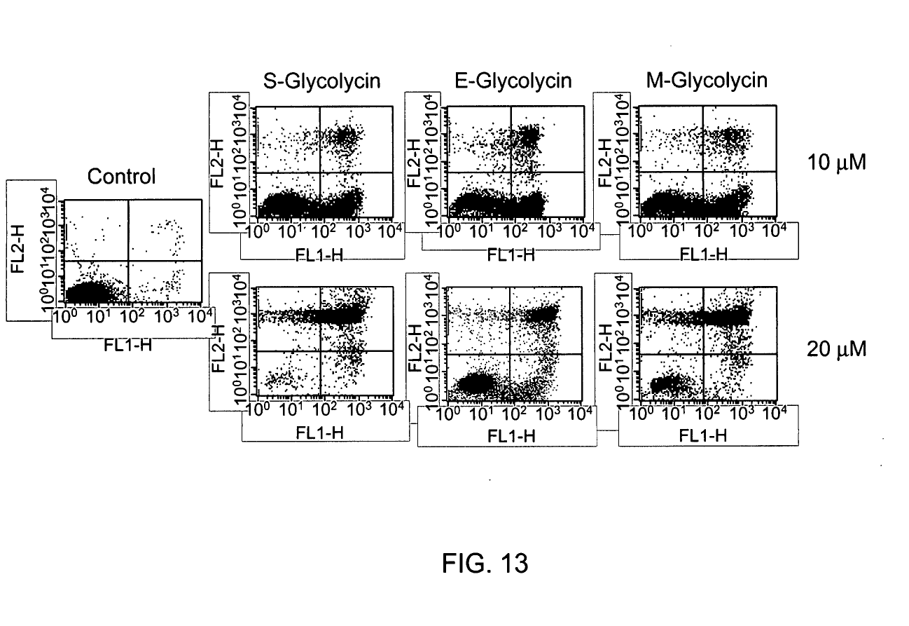

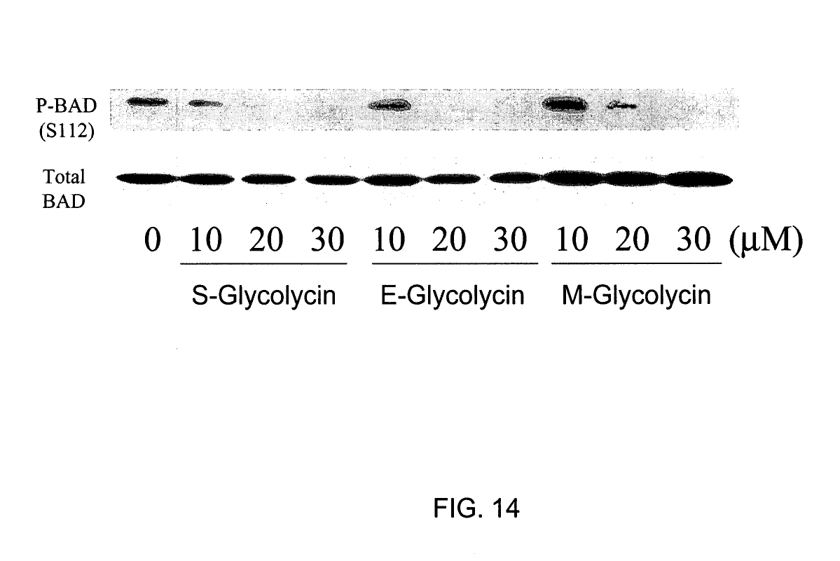

E-Glycolycin and M-Glycolycin are esters and chemically less polarized than 3-bromo-2-oxopropionate, and they are readily permeable through the cellular membranes. Once inside the cells, they can be cleaved by esterase, generating the active component 3-bromo-2-oxopropionate and the respective alcohol (ethanol or methanol). The accumulation of intracellular 3-bromo-2-oxopropoinate effectively inhibits glycolysis, leading to severe depletion of cellular ATP and massive killing of cancer cells associated with dephosphorylation of the pro-apoptotic protein BAD, as illustrated in FIGS. 12-14.

### Example 3

### S-Glycolycin(1) Chemistry

S-Glycolycin (Sodium 3-bromo-2-oxopropionate carbonic acid) has the following chemical formula: NaC₃H₃BrO₃.H₂CO₃ (Formula weight: 251).

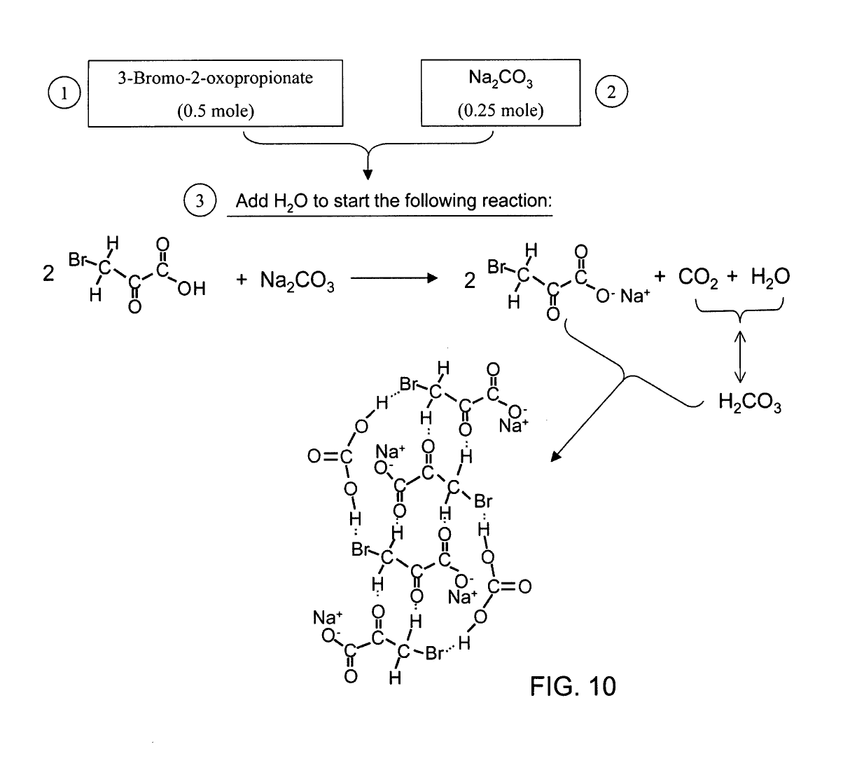

As illustrated in FIG. 10, in aqueous (water) solution the components of S-Glycolycin, sodium 3-bromo-2-oxopropionate (NaC₃H₃BrO₃) and carbonic acid (H₂CO₃), may form hydrogen bonds and stabilize the compound. The interaction between the hydrogen of carbonic acid and the halogen of 3-bromo-2-oxopropionate minimizes the hydrolysis of the Br-C3 bond. However, carbon dioxide (CO₂) may be released from Glycolycin solution, and thus change the molar ratio of sodium 3-bromo-2-oxopropionate (NaC₃H₃BrO₃) and carbonic acid (H₂CO₃). The amount of CO₂ released will depend on the temperature and air pressure (PCO₂) within the container.

In a particular aspect of the invention, the sodium 3-bromo-2-oxopropionate portion of S-Glycolycin is the component responsible for its anticancer activity, whereas the carbonic acid portion of Glycolycin is critical for enhancing the stability and activity of the drug. The present inventors have discovered that the absence of carbonic acid renders 3-bromo-2-oxopropionate unstable in solution at neutral pH, and that sodium 3-bromo-2-oxopropionate (the neutralized reaction product of 3-bromo-2-oxopropionic acid and NaOH) without carbonic acid is significantly less active than Glycolycin. These differences in chemical/biological properties are further described under Sections (II) and (III). Thus, S-Glycolycin should be stored as dry powder (see below), and its solution should be kept in a sealed container to minimize the release of CO₂.

(2) Methods of Preparation

S-Glycolycin can be prepared using commercially available starting materials, in some embodiments. In specific embodiments of the present invention, the starting materials include 3-bromo-2-oxopropionic acid (3-bromopyruvate), sodium carbonate, sodium bicarbonate, sterilized CO₂ gas, and double-distilled water. The present inventors have designed and tested the following methods for preparation of S-Glycolycin. These methods have the advantage of being simple, easy to scale up for pharmaceutical production, and cost-effective.

Method 1:

(1) Dissolve 83.5 grams of 3-bromo-2-oxopropionic acid in double-distilled water (final volume, 1000 ml). This will result in a 0.5 M solution (solution A).

(2) Dissolve 26.5 grams of sodium carbonate (Na₂CO₃) in 500 ml of double-distilled water. This will result in a 0.5 M Na₂CO₃ solution (solution B).

(3) Mix solution A and solution B at the volume ratio of 2:1 (A:B) to start the reaction illustrated in FIG. 10. When the reaction is completed, the pH of the product solution should be near neutral.

(4) Adjust the reaction product to pH 7.0 using either solution A (if the initial pH of the product solution is greater than 7.0) or solution B (if the initial pH is below 7.0). The product is a 0.33 M Glycolycin solution.

(5) Immediately filtrate the neutralized product, and transfer the sterilized product to a container so that the drug solution occupies 90% of the container volume. Fill the remaining 10% volume with sterilized CO₂, and then seal the container.

In an alternative procedure for producing S-Glycolycin by this method, there is the following:

(1) Mix 16.7 grams of 3-bromo-2-oxopropionic acid and 5.3 grams of sodium carbonate (Na₂CO₃) in a container. This powder mix can be stored in a dry container, and dissolved in water to prepare a solution at appropriate concentration before use (see below).

(2) Add 170 ml of double-distilled water to the drug powder, stir to dissolve the compounds.

(3) Adjust the pH of the solution to 7.0 using solution A (if the initial pH is greater than 7.0) or solution B (if the initial pH is below 7.0) as described above. Add double-distilled water to adjust the final volume to 200 ml. The product is a 0.5 M Glycolycin solution.

(4) Immediately filtrate the neutralized product, and transfer the sterilized product to a container for immediate use.

An additional exemplary method for generating S-Glycolycin is as follows:

(1) Dissolve 83.5 grams of 3-bromo-2-oxopropionic acid in 1000 ml of double-distilled water to make a 0.5 M solution (solution A).

(2) Dissolve 42.0 grams of sodium bicarbonate (NaHCO₃) in 1000 ml of double-distilled water. This will result in a 0.5 M NaHCO₃ solution (solution C).

(3) Mix equal volumes of solution A and solution C to start the reaction.

(4) Adjust the pH of the reaction product to 7.0, using either solution A (if the initial pH is greater than 7.0) or solution C (if the initial pH is below 7.0). The product is a 0.25 M solution of Glycolycin.

(5) Immediately filtrate the neutralized product, and transfer the sterilized product to a container so that the drug solution occupies 90% of the container volume. Fill the remaining 10% volume with sterilized CO₂ before sealing the container.

An alternative procedure for generating Glycolycin by this method includes:

(1) Mix 16.7 grams of 3-bromo-2-oxopropionic acid and 8.4 grams of sodium bicarbonate (NaHCO₃) in a container. This powder mix can be stored in a dry container, and dissolved in water to prepare a solution at appropriate concentration before use (see below).

(2) Add 170 ml of double-distilled water to the drug powder, stir to dissolve the compounds.

(3) Adjust the pH of the solution to 7.0 using solution A (if the initial pH is greater than 7.0) or solution C (if the initial pH is below 7.0). Add double-distilled water to adjust the final volume to 200 ml. The product is 0.5 M Glycolycin solution.

(4) Immediately filtrate the neutralized product, and transfer the sterilized product to a container for immediate use.
(3) Stability

The solution of 3-bromo-2-oxopropionic acid (3-bromopyruvate), the primary starting material of Glycolycin, is extremely acidic and thus limits its direct use for clinical treatment. The present inventors discovered that a simple neutralization of 3-bromopyruvate solution with sodium hydroxide (NaOH) to pH 7.0 yields an unstable product:
Br—CH₂—CO—COOH+NaOH→Br—CH2-CO—COO—Na++H₂O

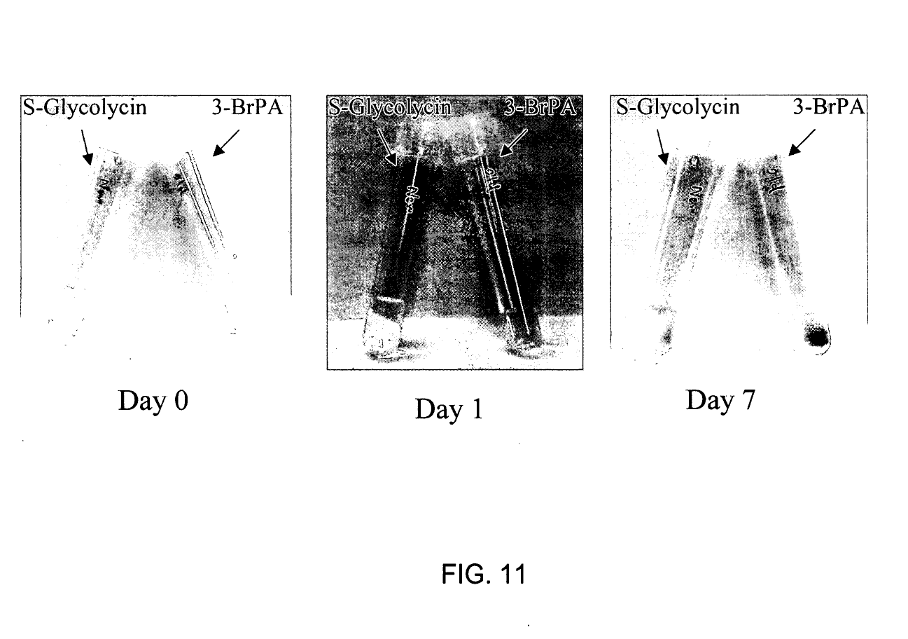

At neutral pH, the solution of 3-bromopyruvate sodium salt (Br—CH₂—CO—COO⁻Na⁺) shows brownish/yellow color within several hours of preparation, probably due to the hydrolysis of Br-C3 bond and the generation of unstable bromic acid. This discoloration becomes more severe with time (even when stored at 4° C.), accompanied by a significant loss of its cytotoxic activity. There is discoloration of 3-BrPA on at least day 1 and day 7. In contrast, the solution of Glycolycin is stable when kept at 4° C. without significant loss of biological activity (FIG. 11 and Table 1).

TABLE 1
Comparison of in vitro cytotoxic activity of Glycolycin and sodium
3-bromopyruvate (3-BrPA) at pH 7.0 before and after they were
stored at 4° C. for 3 weeks. Both compounds were tested by
MTT assay under identical conditions (HL-60 cells; 72 h drug
incubation; 4 h incubation with the MTT reagent).

| | | % Cell Survival | |
|---|---|---|---|
| | | Fresh drug solution | Drug stored for 3 weeks |
| Glycolycin: | 5 μM | 19.3 ± 5.7 | N.D. |
| | 10 μM | 2.5 ± 0.8 | 3.0 ± 1.3 |
| | 30 μM | 3.7 ± 0.6 | 2.9 ± 1.2 |
| | 100 μM | 3.5 ± 1.7 | 2.5 ± 0.9 |
| 3-BrPA: | 5 μM | 99.8 ± 1.9 | N.D. |
| | 10 μM | 59.2 ± 4.1 | 70.0 ± 9.8 |
| | 30 μM | 3.0 ± 1.4 | 87.0 ± 23 |
| | 100 μM | 2.5 ± 0.8 | 2.3 ± 0.3 |

S-Glycolycin was more active than 3-bromopyruvate in killing cancer cells. Also, there was a substantial loss of activity after 3-bromopyruvate solution was kept at 4° C. for 3 weeks. The activity of Glycolycin solution remained stable after at least 3 weeks of storage.

### Example 4

### Glycolysis Inhibition and Anticancer Activity of S-Glycolycin And 3-BRPA

The in vitro anticancer activity of S-Glycolycin was characterized in comparison with its chemical precursor 3-bromo-2-oxopropionic acid (3-bromopyruvate). It has been known for more than 20 years that 3-bromopyruvate is an alkylating agent, which inactivates several enzymes including pyruvate dehydrogenase and hexokinase, and inhibit glycolysis (Tunnicliff et al., 1978; Lowe et al., 1984; Ko et al., 2001). However, the instability of 3-bromopyruvate limits its use as an anticancer drug for clinical applications. In contrast, S-Glycolycin exhibits greatly improved stability and significantly more potent anticancer activity, given its ability to inhibit glycolysis and preferentially deprive cancer cells of ATP. Because the mechanisms of action of S-Glycolycin and 3-bromopyruvate are similar (inhibition of glycolysis and depletion of cellular ATP), their activity were evaluated in parallel studies. The relevant data are shown in the following figures.

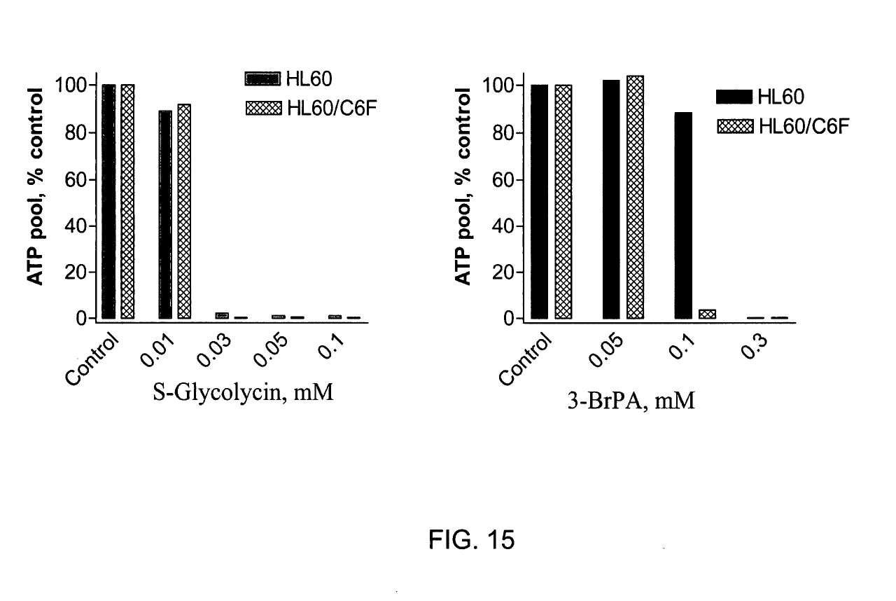

FIG. 15 demonstrates depletion of cellular ATP pool by S-Glycolycin and 3-bromopyruvate (3-BrPA). Cells were incubated with the indicated concentrations of S-Glycolycin or 3-BrPA for 12 h. Cellular ATP was extracted by 0.4 N PCA, neutralized with KOH, and quantified by HPLC analysis. The results indicate that S-Glycolycin is more effective than 3-bromopyruvate in depleting ATP pool in human leukemia HL-60 cell line and its respiration-deficient subclone HL-60/C6F cells. Incubation of cells with 0.03 mM S-Glycolycin completely depleted cellular ATP in both cell lines, while it required a 10-fold greater concentration (0.3 mM) of 3-BrPA to achieve the same degree of ATP depletion.

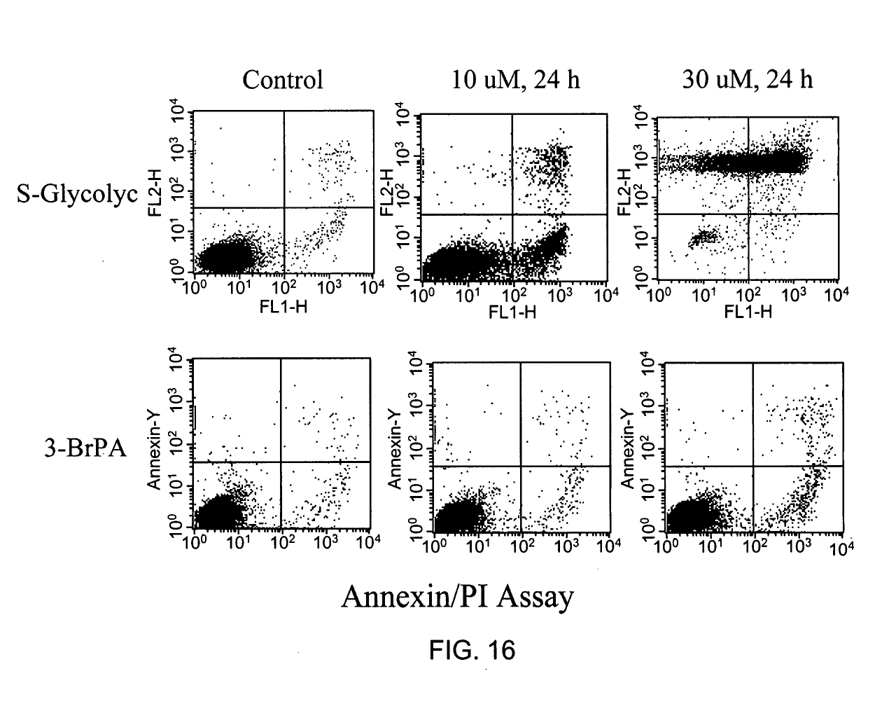

Induction of apoptosis by S-Glycolycin and 3-bromopyruvate (3-BrPA) is demonstrated in FIG. 16. HL-60 cells were incubated with the indicated concentrations of S-Glycolycin or 3-BrPA for 24 h. Apoptosis was measured by double staining with annexin-V and PI, followed by flow cytometry analysis. The data indicate that S-Glycolycin is significantly more potent than 3-BrPA in killing leukemia cells. Incubation of HL-60 cells with 30 μM of S-Glycolycin caused more 90% cells to undergo apoptosis in 24 h, whereas the same concentration of 3-BrPA killed less than 20% of the cells within the same time period.

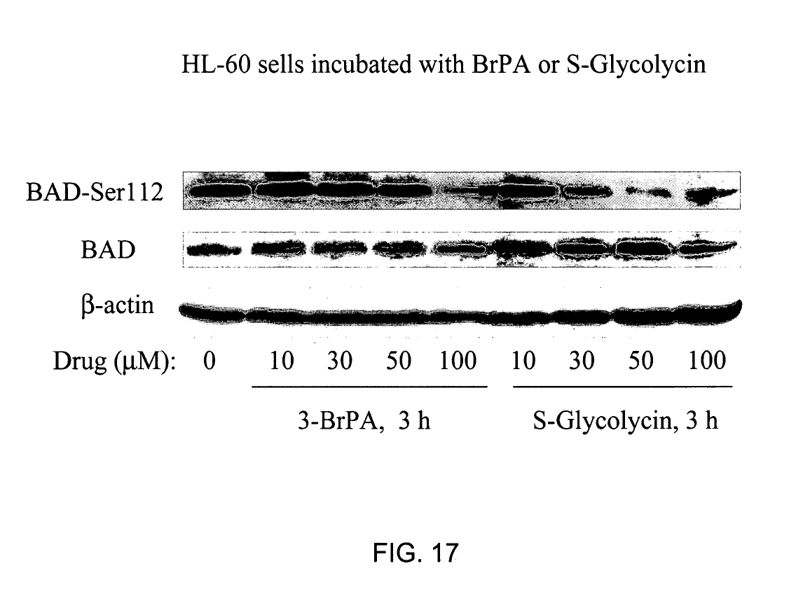

FIG. 17 shows the effect of S-Glycolycin and 3-bromopyruvate on the expression of pro-apoptotic factor BAD and its phosphorylation status (Ser112). HL-60 cells were incubated with the indicated concentrations of each compound for 3 h. BAD and its phosphorylation at Ser112 were measured by western blot analysis, using the respective antibodies. β-Actin was also blotted as the protein loading control. The results indicate that S-Glycolycin is more effective than 3-BrPA in causing dephosphorylation of BAD, which led to cytochrome c release from the mitochondria to cytosol and activation of the apoptotic cascade.

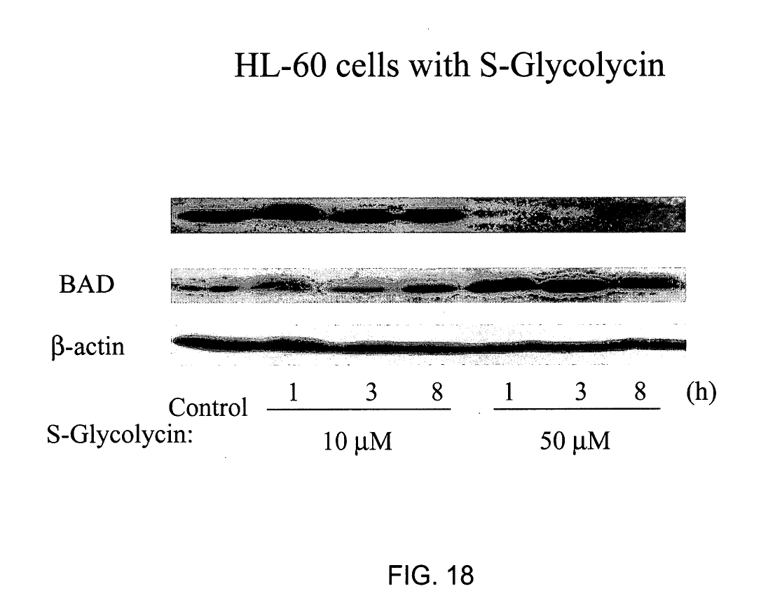

A time- and dose-dependent effect of S-Glycolycin on the expression level of pro-apoptotic factor BAD and its phosphorylation at Ser112 is provided in FIG. 18. HL-60 cells were incubated with the indicated concentrations of S-Glycolycin for various times. Total BAD protein and its phosphorylation at Ser112 were measured by western blot analysis using the respective antibodies. β-actin was also blotted as the protein loading control. Ten μM of S-Glycolycin did not cause any significant change in BAD, whereas 50 μM of the drug caused almost complete dephosphorylation of BAD. Note that severe ATP depletion can be induced by S-Glycolycin at 30-50 μM, but not at 10 μM (FIG. 15). Thus, it indicates that the ability of S-Glycolycin to cause dephosphorylation of BAD is correlated with depletion of the cellular ATP pool.

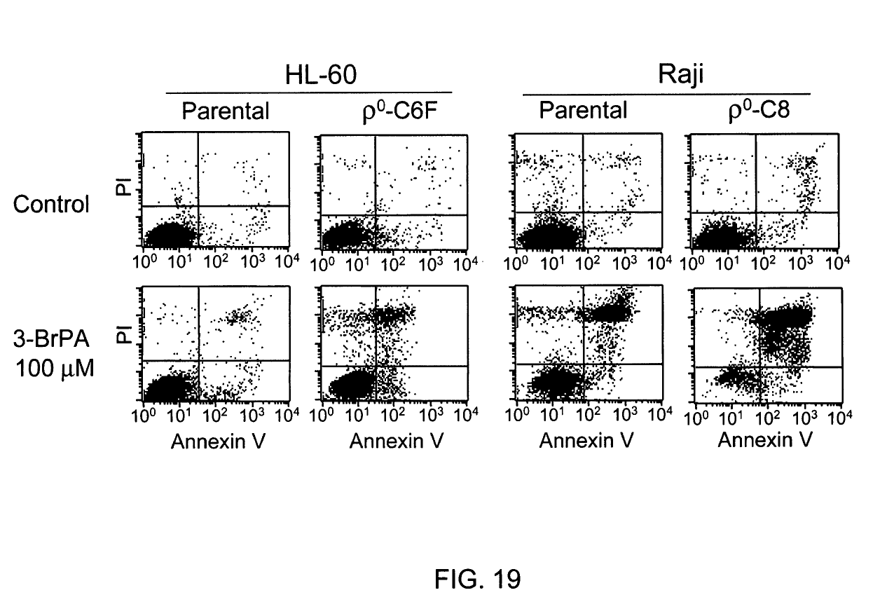

FIG. 19 shows that inhibition of glycolysis leads to more effective killing of cancer cells with respiration defect. Initial studies have been performed to test the sensitivity of respiration-competent leukemia cells (parental HL-60) and lymphoma cells (parental Raji) in comparison with their respiration-deficient clones HL-c6/ρ⁰-C6F and Raji-C6. The mitochondria-defect cell lines were established by the present inventors and others (Pelicano et al., 2003). Cells were incubated with the indicated concentrations of 3-BrPA, and cytotoxicity was measured by two different assays: annexin-V/PI staining and colony formation assay. The results showed that compared to the parental cells, the mitochondria-defect cells are more sensitive to 3-BrPA due to their increased dependence on glycolysis.

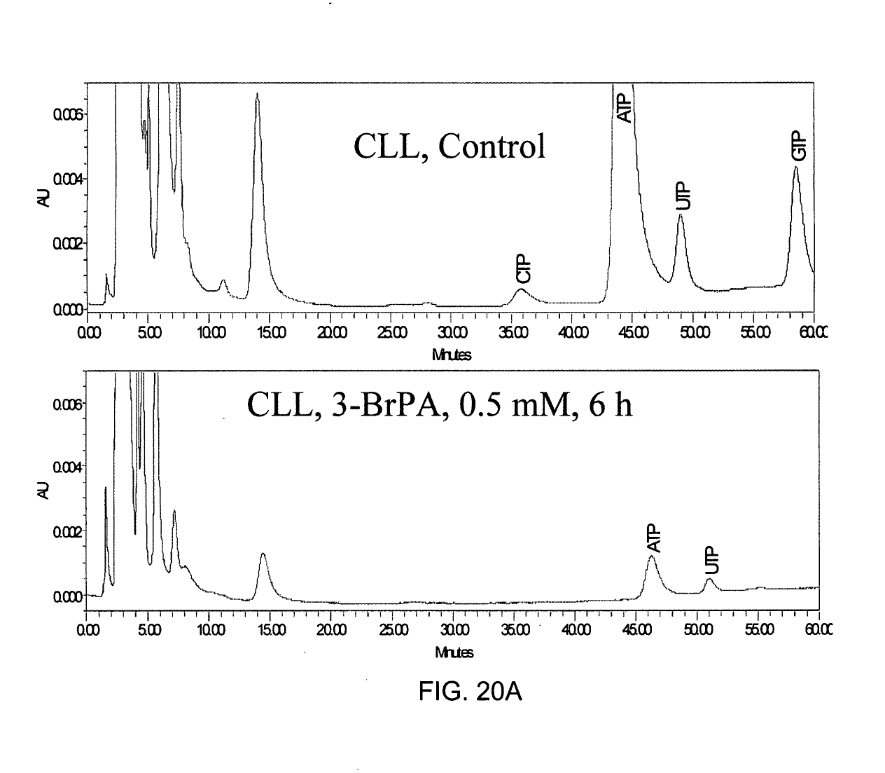

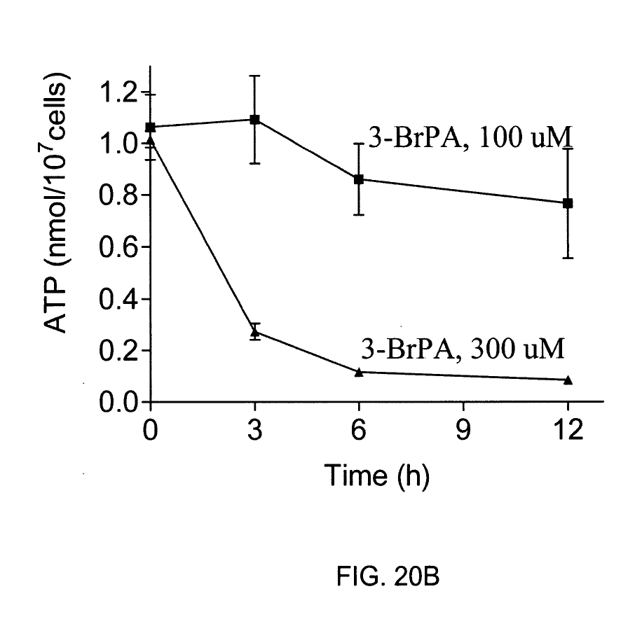

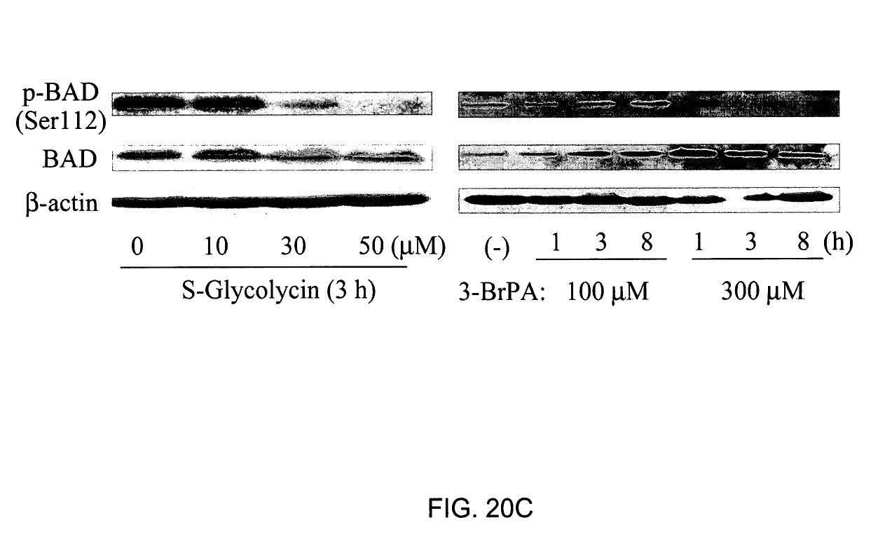

Depletion of cellular ATP and induction of BAD dephosphorylation by 3-BrPA and S-Glycolycin in primary leukemia cells isolated from patients with chronic lymphocytic leukemia (CLL) is demonstrated in FIG. 20. Examples of HPLC analysis of ATP are shown on the top two panels in FIG. 20A. Top panel, standard of nucleotide triphosphates (NTPs); middle panel, extracts from control CLL cells; lower panel, extracts from CLL cells treated with 3-BrPA. The ATP peak is shown in red. Western blot analysis demonstrated that S-Glycolycin is more active than 3-BrPA in causing BAD dephosphorylation in primary CLL cells. MTT assay indicates that S-Glycolycin is more potent than 3-BrPA in killing CLL cells as measured by IC₅₀ values.

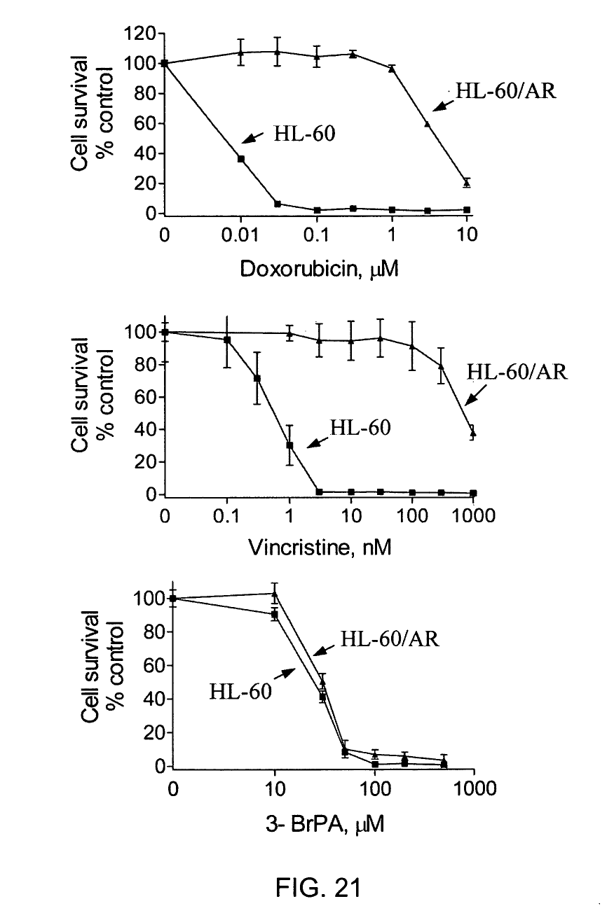

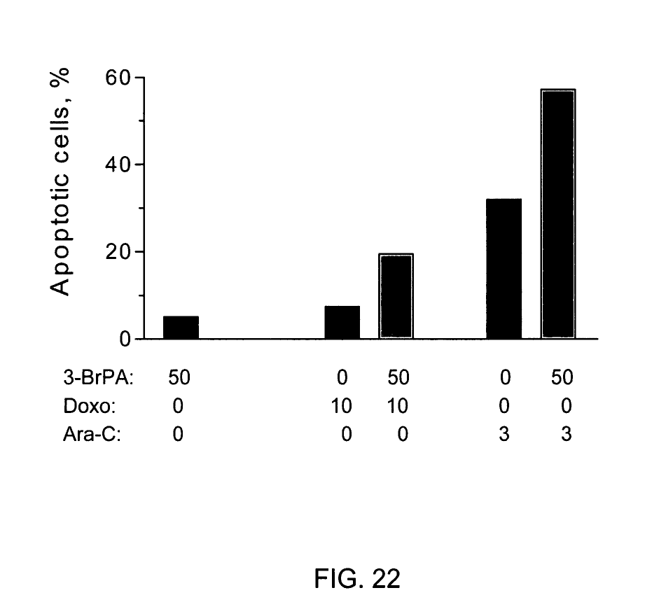

FIG. 21 demonstrates overcoming drug resistance by inhibition of glycolysis and depletion of cellular ATP. HL-60 cell line and its drug-resistant clone HL-60/AR were incubated with the indicated concentrations of doxorubicin, vincristine, and 3-BrPA for 72 h. Cytotoxicity was measured by MTT assay. The results show that HL-60/AR cells are resistant to doxorubicin and vincristine by more than 100 fold compared to the parental HL-60 cells. However, both cell lines are equally sensitive to 3-BrPA. Based on the teachings provided herein, studies may be performed to determine the sensitivity of HL-60/AR cells to Glycolycin. FIG. 22 further shows that combination of 3-BrPA with ara-C or doxorubicin can significantly enhance the cytotoxic activity against the multi drug resistant HL-60/AR cells.

### Example 5

### Effect of S-Glycolycin on Multi-Drug Resistant Leukemia Cells

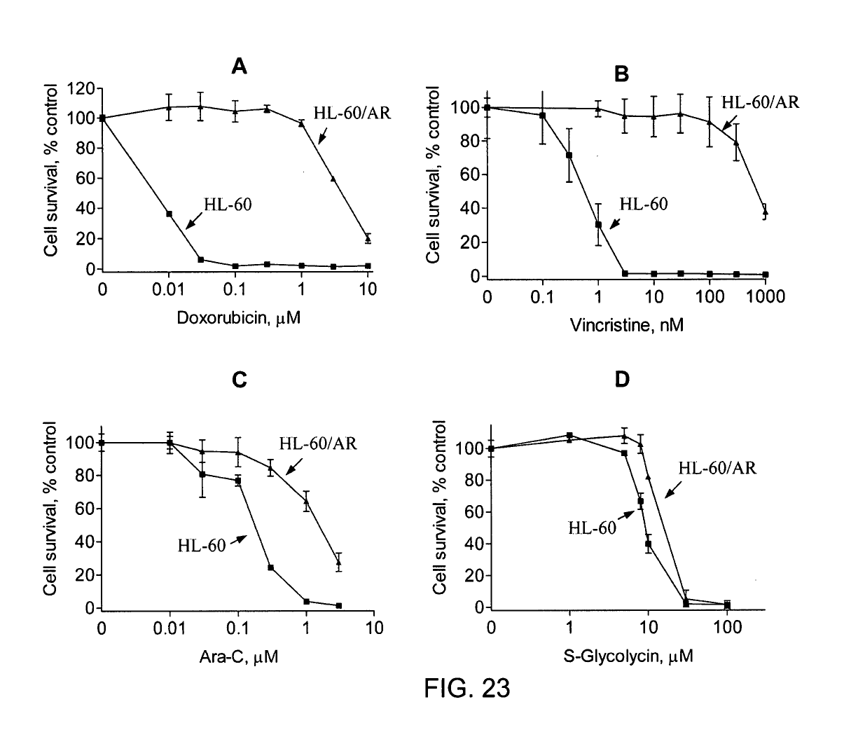

A subclone of human leukemia cells HL-60/AR highly resistant to doxorubicin and vincristine was used to test the ability of S-Glycolycin to kill the multi-drug resistant cells in comparison with the parental HL-60 cells. Ara-C, a drug commonly used in the frontline treatment of leukemia, was used in parallel experiments. Cytotoxicity was measured by MTT assay. As illustrated in FIG. 23, the results show that HL-60/AR cells are highly resistant to doxorubicin (A) and vincristine (B), and moderately resistant to ara-C(C) compared to the parental HL-60 cells. However, both HL-60/AR and HL-60 cells are equally sensitive to S-Glycolycin (D).

### Example 6

### Animal Studies

Animal studies have been performed in mice using the following conditions: Animal: Nude mice, body weight 20-30 grams. (1) Glycolycin dosage: Glycolycin 6 mg/kg, i.p. daily for three days. (2) E-Glycolycin dose: 5 mg/kg, i.v. daily for 5 days. (3) S-Glycolycin dose: S-Glycolycin 5 mg/kg, i.v., three times per week, M/W/F). A second strain of mice (C3H-Hej) were injected with Glycolycin: 10 mg/kg/day, i.v., daily for 5 days.

No obvious acute toxicity has been observed under these conditions. These drug dosages are expected to produce plasma drug concentrations of at least 30 μM, which is above the IC₅₀ of Glycolycin against the cancer cells tested in vitro.

### Example 7

### Exemplary Combination Formulations: Glycolycin-P and Glycolycin-G

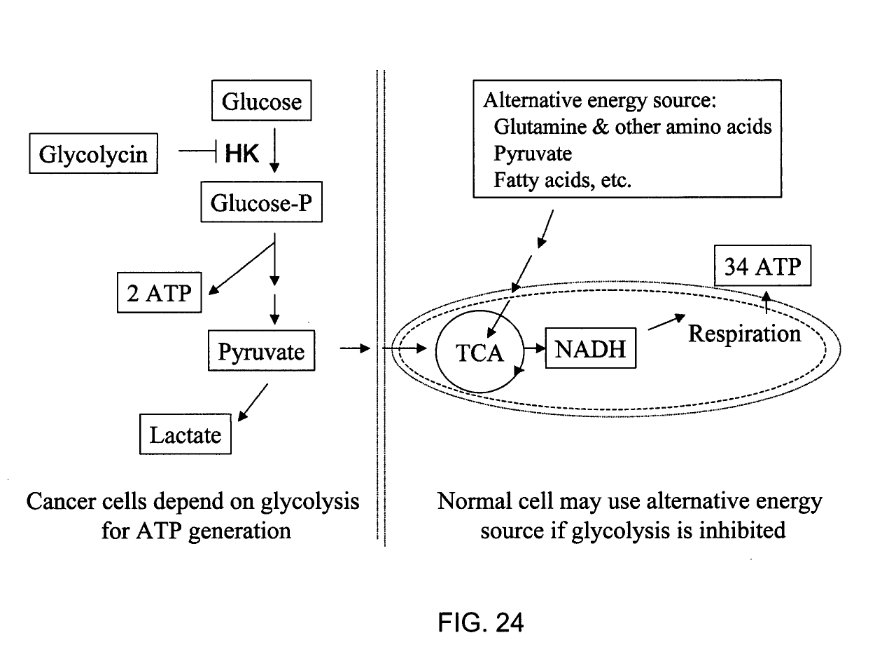

The difference in energy metabolism between cancer cells and normal cells provides a biochemical basis to design drug combination formulations to improve therapeutic activity of Glycolycin and reduce its toxicity to normal cells. Because cancer cells have respiration injury and are more dependent on glycolysis to generate ATP (the Warburg effect), inhibition of glycolysis by Glycolycin is expected to effectively kill cancer cells. In contrast, normal cells with competent mitochondrial respiration are able to use alternative energy sources such as amino acids, fatty acids, and metabolic intermediates (e.g. pyruvate) to generate ATP if glycolysis is inhibited. Thus, supplement of such metabolic intermediates in combination with Glycolycin is expected to provide protection for the normal cells, and thus reduce unwanted toxicity. That is, due to their ability to utilize the indicated metabolic intermediates for generation of ATP through the TCA cycle and mitochondrial respiration, normal cells have enhanced toleration of glycolysis inhibition if such alternative energy sources are provided. The rationale for such drug combinations is illustrated in FIG. 24.

(1) Glycolycin-G comprises the combination of Glycolycin or its derivative and glutamine at an appropriate molar ratio. Glutamine can be used as an energy source via the following pathway:

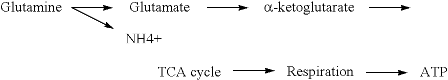

(2) Glycolycin-P comprises the combination of Glycolycin or its derivative and pyruvate at an appropriate molar ratio. Pyruvate can be used as an energy source via the following pathway:

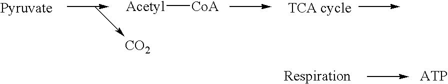

(3) Other combinations comprise Glycolycin+glutamine+pyruvate; Glycolycin+fatty acids; and Glycolycin+fatty acids+glutamine, for example.

### Example 8

### Effect of Glutamine on S-Glycolycin Cytotoxicity in Respiration-Competent Cells and Respiration-Defective Cells

In specific embodiments of the present invention, cells with competent mitochondrial respiration function utilize alternative energy sources, such as amino acids, fatty acids, and/or other metabolic intermediates to generate ATP when glycolysis is inhibited. It is expected that supplementation of alternative energy sources, such as glutamine, will reduce the cytotoxic effect of Glycolycin on cells with normal mitochondrial function. In contrast, such nutritional supplementation would not protect cancer cells with defective mitochondria due to their inability to effectively use the alternative energy source.

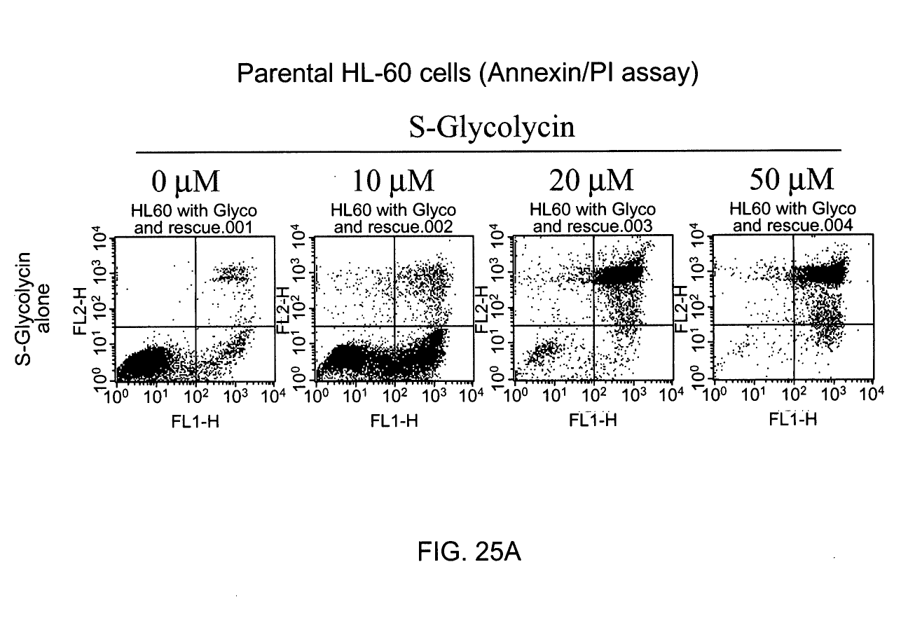

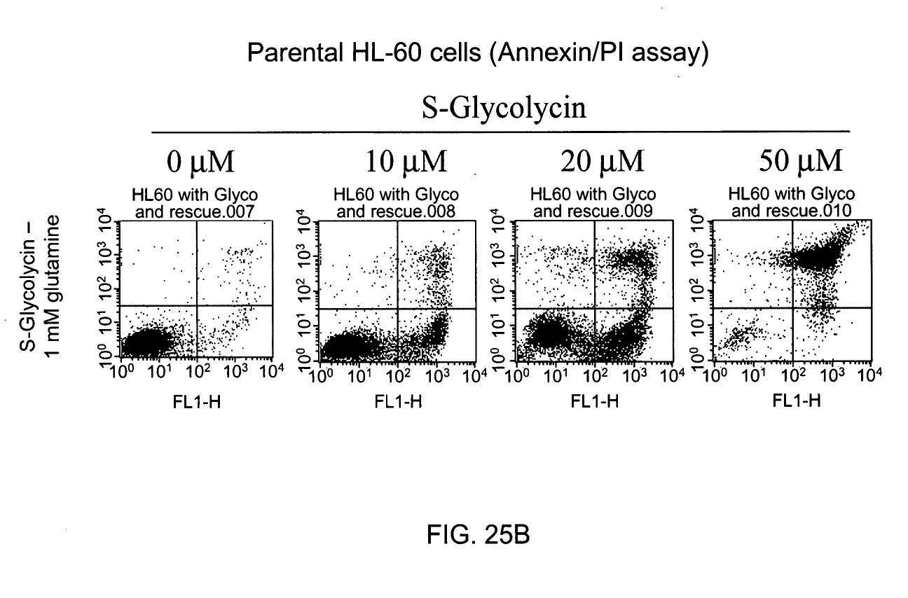

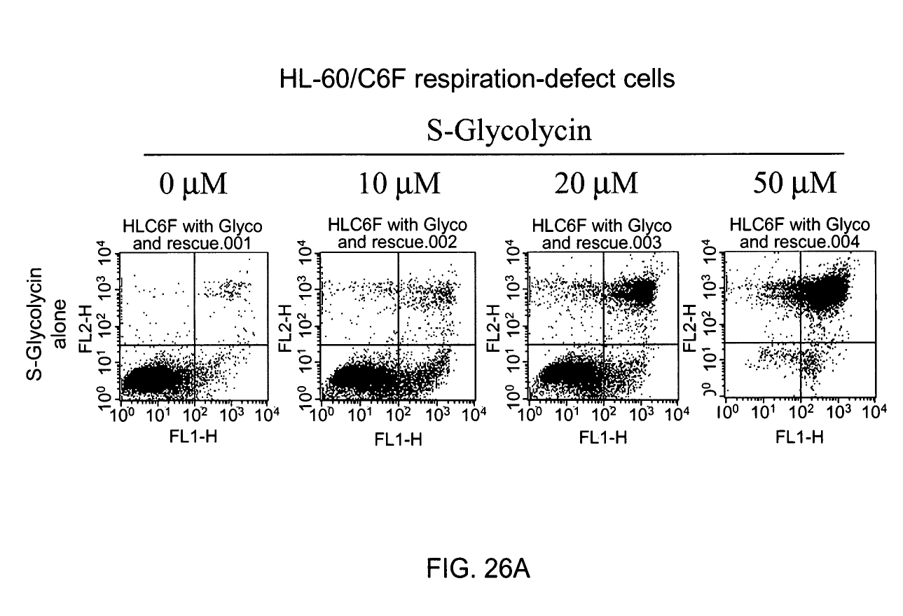

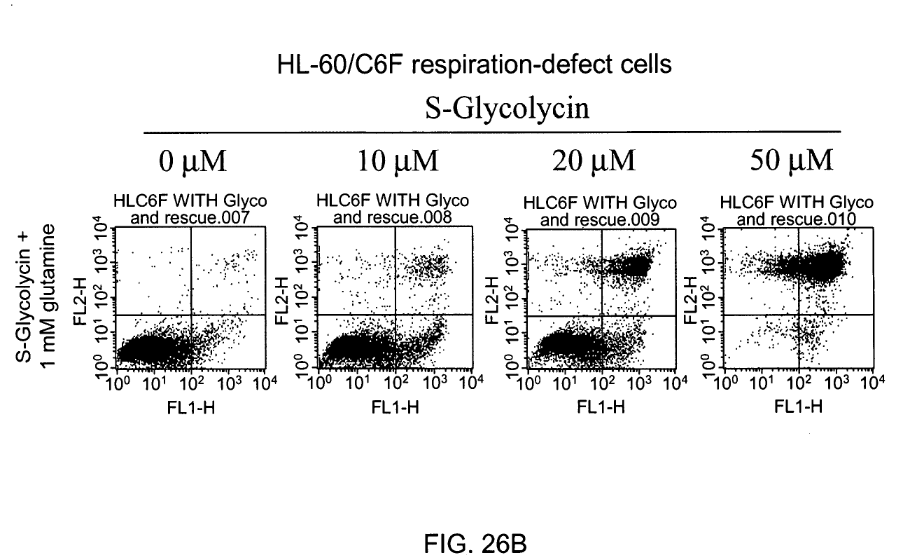

The effect of glutamine on the cytotoxic activity of S-Glycolycin in wild-type HL-60 cells and respiration-deficient C6F cells was compared. Parental HL-60 cells were incubated with the indicated concentrations of S-Glycolycin in the presence or absence of 1 mM glutamine for 2 h, washed, and re-cultured in fresh medium. Apoptosis was measured at 24 h. The results show that addition of glutamine to the cell culture provided a partial protection to the parental HL-60 cells treated with Glycolycin (FIG. 25), whereas no protection was observed with the respiration-deficient C6F cells (FIG. 26). In FIG. 26, the respiration-deficient cells (HL-60-C6F) were incubated with the indicated concentrations of S-Glycolycin in the presence or absence of 1 mM glutamine for 2 h, washed, and re-cultured in fresh medium. Apoptosis was measured at 24 h. Thus, glutamine supplement offers some degree of protection for cells with competent mitochondrial function.

### Example 9

### Cytotoxic Activity of S-Glycolycin against Human Colon Cancer (HCT116) and Brain Tumor (U87MG) Cells

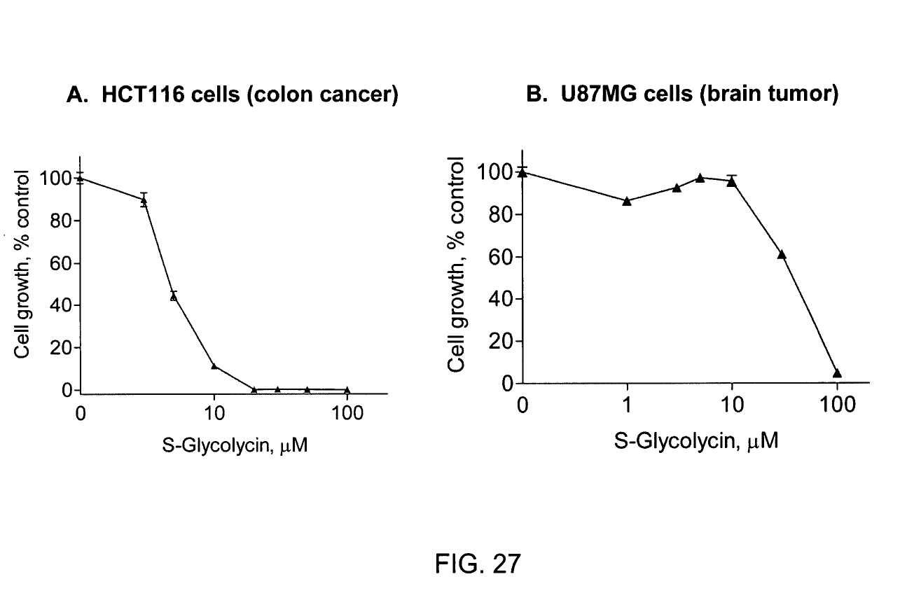

The effect of Glycolycin on human colon cancer cells (HCT116) and human brain tumor cells (U87MG) were tested using MTT assays for a 72 hour incubation. As shown in FIG. 27, HCT116 were sensitive to S-Glycolycin, with the IC₅₀ value of approximately 5-10 μM. The IC₅₀ value of S-Glycolycin for brain tumor U87MG cells is approximately 30 μM. The ability of S-Glycolycin to kill solid tumor cells in vitro suggests that Glycolycin may be useful for the treatment of solid tumors in vivo. Furthermore, a separate experiment demonstrated that colon cancer cells without p53 expression were similarly sensitive to S-Glycolycin, suggesting that inhibition of glycolysis is effective in killing cancer cells with defective p53. This is of clinical significance, since the majority of human cancers are defective in p53 function and show reduced response to radiotherapy and certain anticancer agents.

### Example 10

### Over-Expression of BCL-2 Does Not Protect Cells from the Cytotoxic Effect of Glycolycin

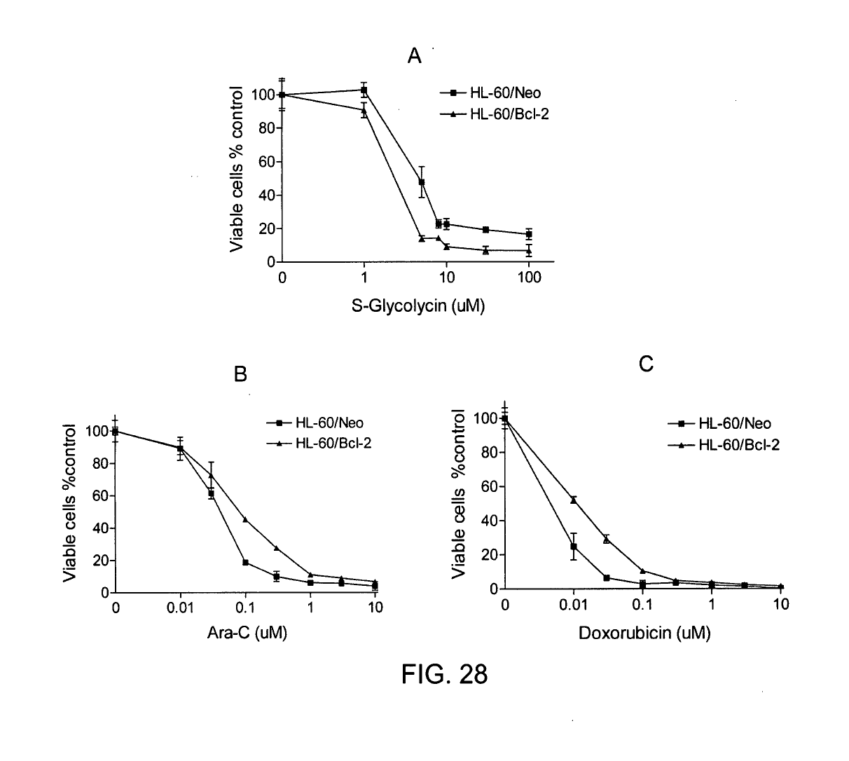

Increased expression of the anti-apoptotic molecule Bcl-2 has been associated with drug resistance in cancer cells. The over-expression of Bcl-2 in HL-60 cells was characterized for providing protection against the cytotoxic effect of S-Glycolycin. The results shown in FIG. 28 demonstrate that cells with high Bcl-2 expression after gene transfection remain sensitive to S-Glycolycin, as compared to the transfected control (Neo). The Bcl-2 transfected cells exhibit reduced sensitivity to ara-C and doxorubicin. Thus, Glycolycin may be used to overcome drug resistance due to Bcl-2 over expression.

### Example 11

### S-Glycolycin is More Effective in Killing Cancer Cells under Hypoxia Condition Then under Normoxia Condition

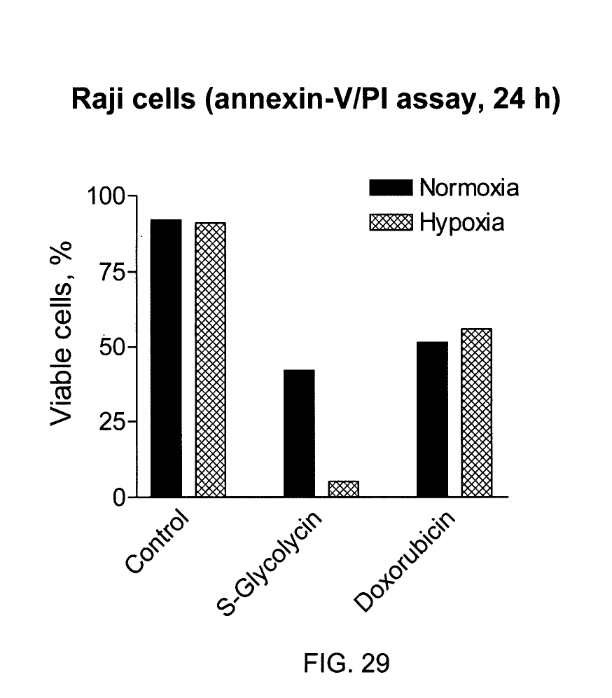

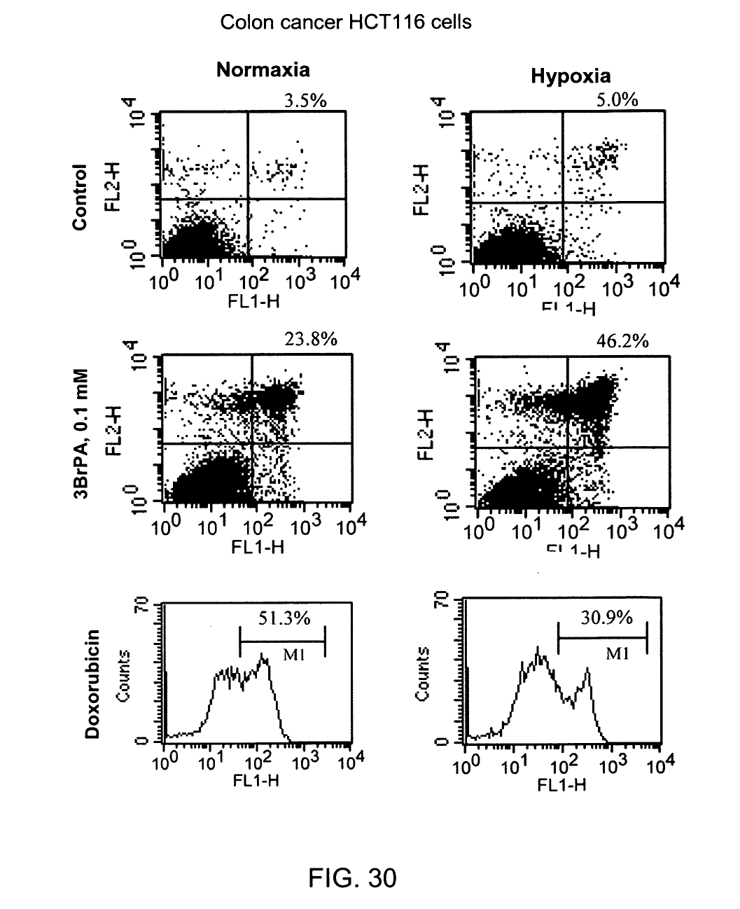

Cancer cells in solid tumors have to adapt to the hypoxia environment due to insufficient blood supply. Under hypoxia conditions, cancer cells use the glycolytic pathway to generate ATP without consuming oxygen. This metabolic adaptation also renders cancer cells relatively resistant to anticancer agents and radiation therapy, partially due to up-regulation of expression of cell survival factors such as HIF-1α. The reduced sensitivity to chemotherapy and radiotherapy due to hypoxia poses a significant problem in clinical treatment of cancer. However, because under hypoxia condition cancer cells dependent on glycolysis for generation of ATP, it is contemplated that such cancer cells are vulnerable to glycolytic inhibition by Glycolycin. Indeed, it has been shown by the current inventors that human lymphoma cells (Raji) are significantly more sensitive to S-Glycolycin under hypoxic conditions than under normoxic conditions (FIG. 29). Similarly, human colon cancer cells (HCT116) are also more sensitive to glycolytic inhibition by 3-BrPA under hypoxic conditions than under normoxic conditions (FIG. 30). In contrast, doxorubicin is less effective under hypoxic conditions (FIGS. 29-30). Thus, it is envisioned that Glycolycin can be use for effective treatment of many solid tumors, either as a single agent or in combination with other anticancer agents.

### Example 12

### Glycolycin Effectively Killing Cancer Cells under Hypoxia Condition in Combination with Ionizing Radiation

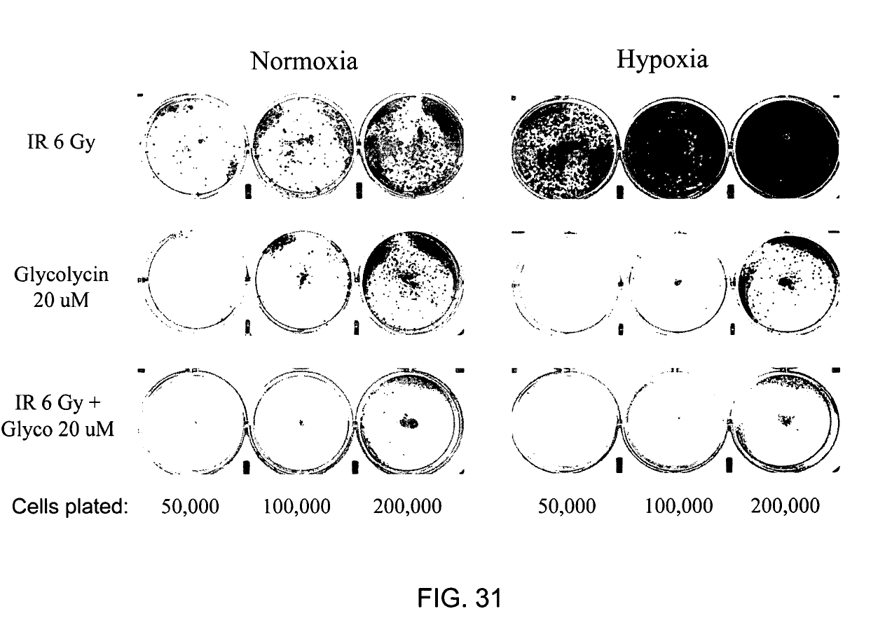

It is known in the art that under hypoxic conditions, cancer cells become less sensitive to radiation therapy, partially due to up-regulation of expression of survival factors and less generation of free radicals without oxygen. However, under hypoxic conditions cancer cells depend on glycolysis for generation of ATP, which is the essential energy source for repair of the DNA damage caused by radiation. Thus, it is contemplated that Glycolycin can effectively deplete cellular ATP pool under hypoxic conditions and enhance the therapeutic activity of radiation. As illustrated in FIG. 31, human colon cancer cells (HCT116) under hypoxic conditions are much less sensitive to γ-radiation (top panel). In contrast, cells in hypoxic conditions are more sensitive to Glycolycin (middle panel). Combination of Glycolycin and radiation almost completely killed all cells plated (up to 200,000 cells/well). Thus, Glycolycin is an effective agent to enhance the effectiveness of radiotherapy.

### Example 13

### Exemplary Glycolycin Ester

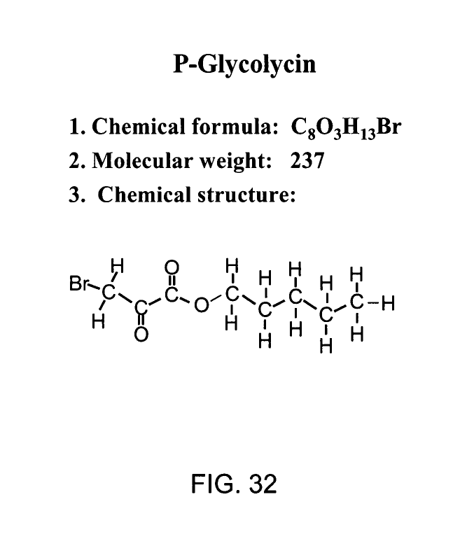

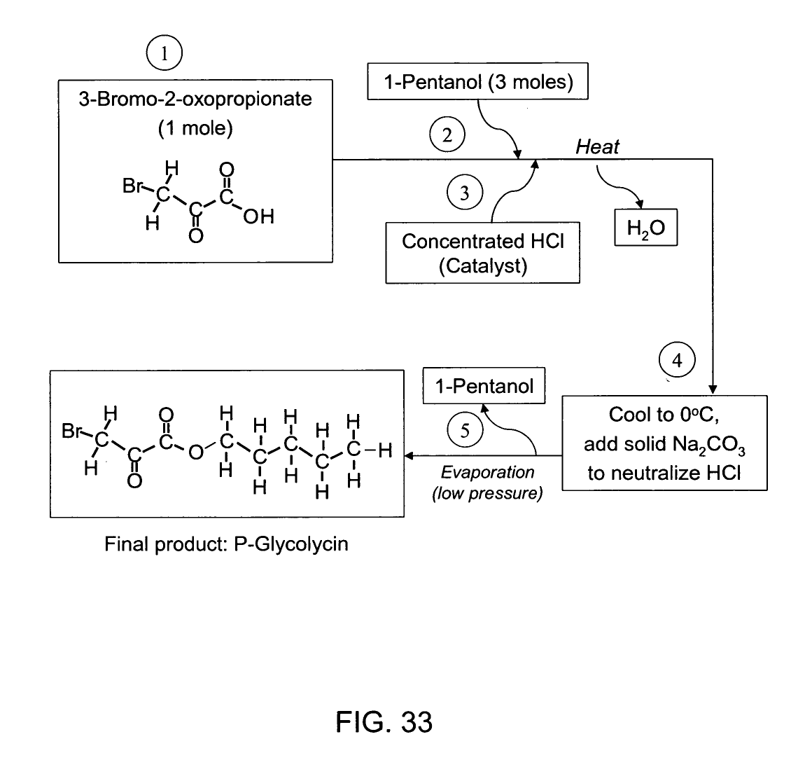

In exemplary embodiments of the invention, there is an exemplary glycolycin ester as provided in FIG. 32, and FIG. 33 shows an exemplary method for preparation of this compound, also referred to as P-glycolycin. In particular, the method is as follows: (1) place 1 mole of 3-bromopyruvate solid in a reaction chamber; (2) add 3 moles of pure 1-Pentanol; (3) Add a small amount of concentrated hydrochloric acid as the catalyst, heat the chamber to 40° C. for 60 min, then cool to 30° C. and let the reaction continue for 8 hours with constant stirring. (4) cool the reaction products to 0° C., add solid Na₂CO₃ to neutralize HCl. (5) evaporate off the excess 1-pentanol.

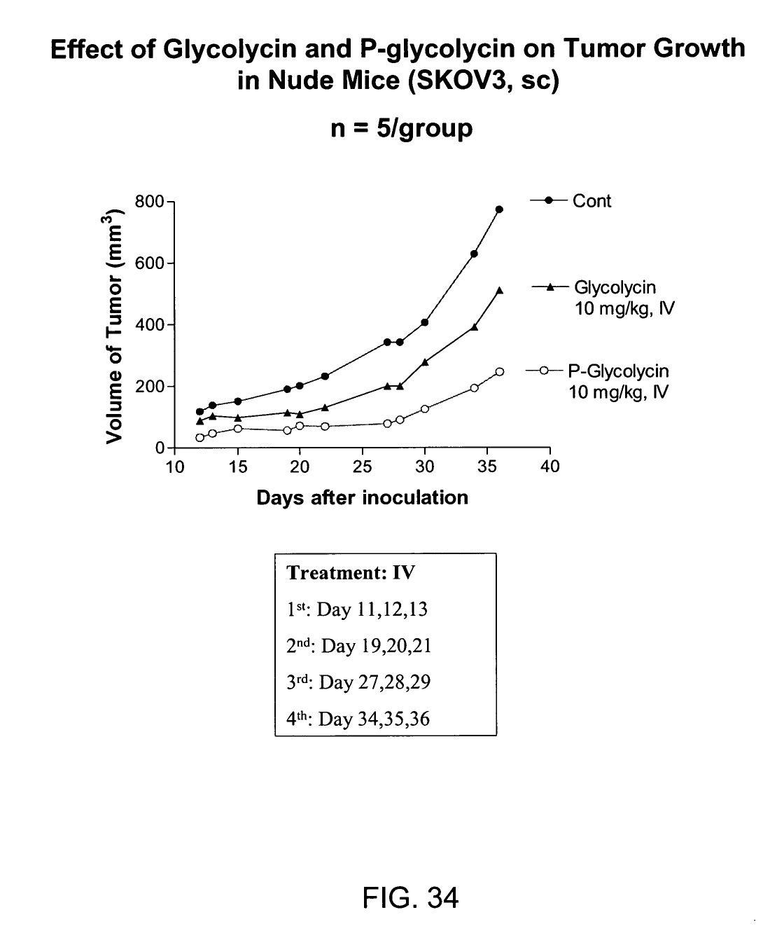

FIG. 34 demonstrates the effect of Glycolycin and P-glycolycin on tumor growth in nude mice. Animals (5 mice/group) were inoculated with human ovarian cancer cells (SKOV3) by s.c. inoculation. Glycolycin and P-glycolycin were administered by i.v. injection using the indicated dose-schedules, starting on day 11 after tumor inoculation.

### Example 14

### Inhibition of MTOR and Glycolysis Leads to Synergistic Cytotoxicity in Lymphoma and Leukemia Cells

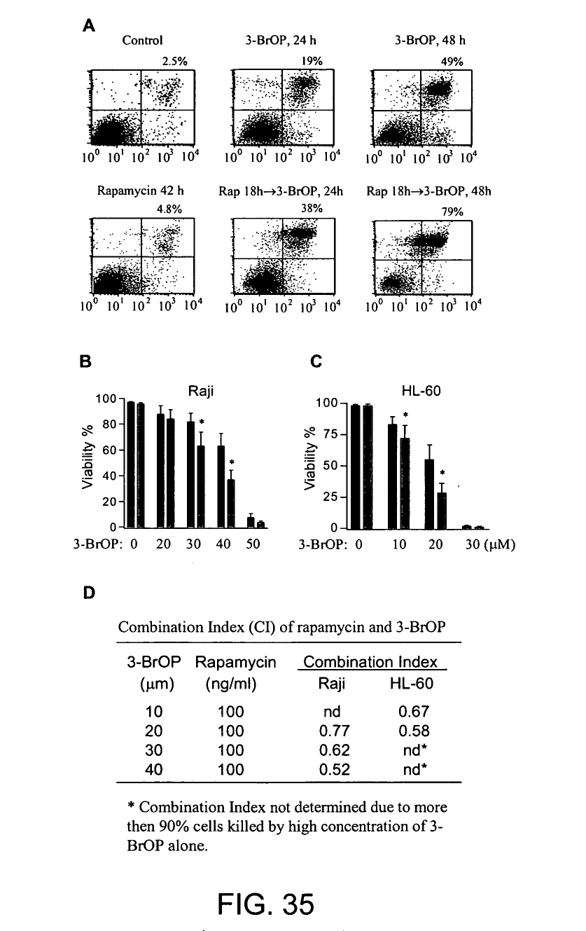

Because the mTOR pathway plays an important role in promoting cellular nutrient metabolism and cell growth, and function to enhance cell survival downstream of the P13K/Akt pathway, the present inventors postulated that a simultaneous inhibition of mTOR and glycolysis, which serve as a major metabolic pathway for ATP generation in cancer cells, would severely impact cellular energy metabolism and have a potent cytotoxic effect against malignant cells. To test this possibility, rapamycin was used as an agent to inhibit mTOR, and glycolycin (3-bromo-2-oxopropionate, abbreviated herein as 3-BrOP) to inhibit glycolysis. 3-BrOP is a cell permeable ester that is hydrolyzed to release 3-bromopyruvate, a potent inhibitor of the glycolytic enzyme hexokinase (Xu et al., 2005; Geschwind et al., 2004; Ko et al., 2001; Ko et al., 2004). As shown in FIG. 35A, treatment of human lymphoma cells (Raji) with 30 μM 3-BrOP resulted in 19% and 49% apoptosis at 24 h and 48 h, respectively, as measured by flow cytometry analysis after the cells were double-stained with annexin-V and PI. Combination of 3-BrOP with 100 ng/ml rapamycin, which by itself alone did not induce significant apoptosis (4.8%), led to a substantial increase of apoptosis, with 38% and 79% apoptotic cells at 24 h and 48 h, respectively.

The above observations prompted the present inventors to use a formal drug combination analysis to determine if rapamycin and 3-BrOP have a synergistic effect. Both Raji cells and HL-60 leukemia cells were used for this study. Cells were treated with various concentrations of 3-BrOP in the presence of a fixed concentration of rapamycin (100 ng/ml) for 24 h, and the percentage of apoptotic cells was quantitated by flow cytometry analysis using annexin-V/PI double staining. As illustrated in FIGS. 35B and 35C, the non-toxic concentration of rapamycin (100 ng/ml) significantly potentiate the cytotoxic activity of 3-BrOP at most concentrations tested in both cell lines. The median-effect analysis program described by Chou and Talalay was then used to calculate the drug combination index (CI) (Chou et al., 1996). In this analysis, an additive effect would produce a CI value of 1, whereas synergistic and antagonistic effects would give CI values of less than 1 and greater than 1, respectively. As shown in FIG. 35D, the CI values were less then 1.0, indicating that the rapamycin and BrOP produced synergistic cytotoxicity when they were used together.

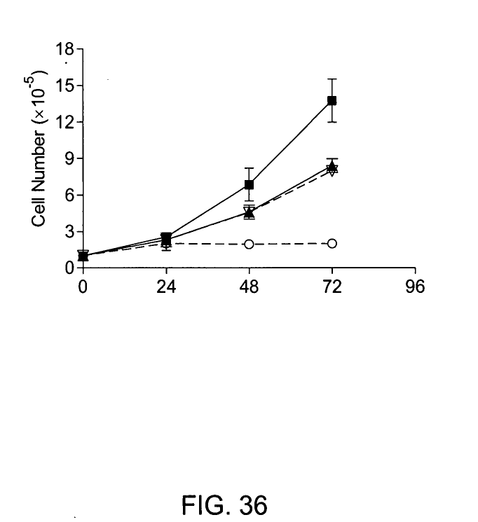

A different assay was then used to further confirm the synergy between rapamycin and 3-BrOP. Raji cells were treated with 30 μM 3-BrOP, 100 ng/ml rapamycin, or their combination, and the numbers of cells were directly counted for consecutive 3 days, using a Coulter Z2 Particle Count & Size Analyzer. As illustrated in FIG. 36, either 3-BrOP or rapamycin alone caused a moderate delay of the cell growth under these experimental conditions, with approximate 35% decrease in cell numbers in each case. It is worth noting that the same concentration of rapamycin (100 ng/ml) did not cause acute cell death in apoptosis assay (FIG. 35), but caused a 35% decrease in cell growth, consistent with the cytostatic effect of rapamycin in cells that preserve cell cycle checkpoint function (Easton and Houghton, 2004; Huang et al., 2003). Importantly, combination of these two agents at sub-toxic concentrations exhibited an almost complete inhibition of cell growth (FIG. 36), confirming their synergistic activity.

### Example 15

### Rapamycin Exhibits No Significant Effect on ARA-C Cytotoxic Activity

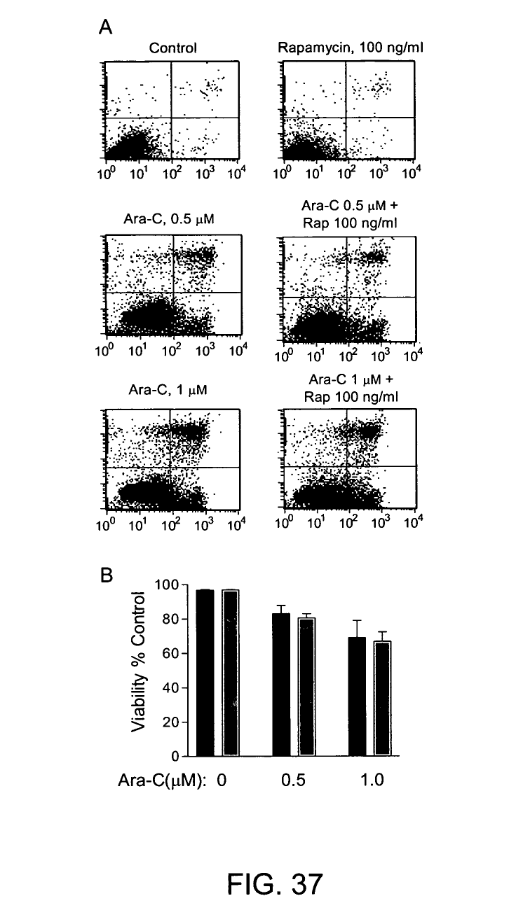

Based on the ability of rapamycin to potentiate the activity of 3-BrOP in human lymphoma and leukemia cells, it was then tested if rapamycin could be used to enhance the cytotoxic activity of ara-C, an effective agent widely used in clinical treatment of leukemia and certain lymphoma. Flow cytometry analysis showed that rapamycin did not significantly affect the cytotoxic activity of ara-C in Raji cells (FIG. 37A). For instance, ara-C at the concentration range of 0.5-1 μM induced apoptosis in 20-35% of the cells. Addition of 100 ng/ml rapamycin led to a similar percent of apoptotic cells (FIG. 37). This is in contract to the results from the 3-BrOP experiments, in which the same concentration of rapamycin increased apoptosis from 19% to 38% (FIG. 35A). Thus, it appears that combination of rapamycin and ara-C may not significantly increase cell killing. Based on this observation, studies were focused on combination of rapamycin with 3-BrOP to further examine the possible mechanisms with respect to ATP metabolism and the relevant molecular events.

### Example 16

### Combination of Rapamycin And Glycolycin (3-BROP) Causes a Synergistic Depletion of Cellular ATP

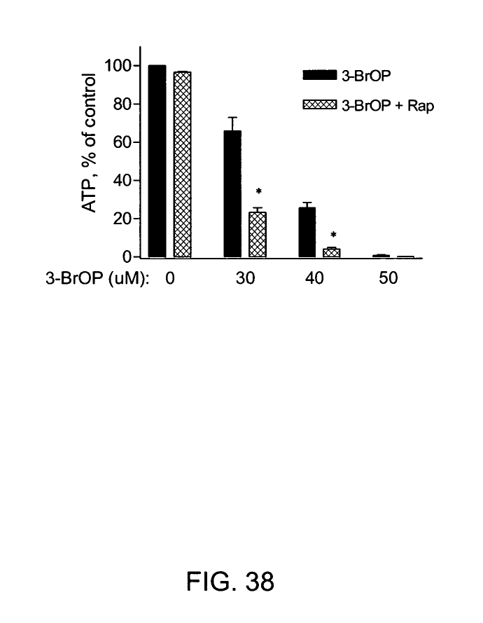

HPLC analysis was then used to determine cellular ATP pool in an attempt to understand the biochemical basis of the synergistic activity of rapamycin and 3-BrOP. Raji cells were treated with various concentrations of 3-BrOP in the presence and absence of rapamycin (100 ng/ml) for 6 h, and cellular ATP and other nucleotide pools were measured by HPLC analysis as described in Examples herein. As illustrated in FIG. 38, treatment of Raji cells with 3-BrOP resulted in a concentration-dependent decrease in cellular ATP pool. Of note, the decrease of ATP pool occurred at 6 h after incubation with 3-BrOP and was well before the appearance of apoptosis, suggesting that the ATP pool reduction was not a consequence of cell death.

Incubation of Raji cells with 100 ng/ml rapamycin alone for 24 h did not cause any detectable change in cellular ATP pool (FIG. 38). However, pre-incubation of Raji cells with this concentration of rapamycin for 18 h followed by a 6-h treatment with 3-BrOP significantly enhanced the depletion of cellular ATP. Because this ATP depletion occurred before apoptosis became detectable, it seems that the enhancement of ATP depletion might be a biochemical mechanism by which rapamycin promote the cytotoxic effect of 3-BrOP. This led the present inventors to further seek possible mechanisms contributing to this increased depletion of ATP pool.

### Example 17

### Rapamycin Significantly Enhances the Ability of Glycolycin (3-BROP) to Inhibit Glucose Uptake

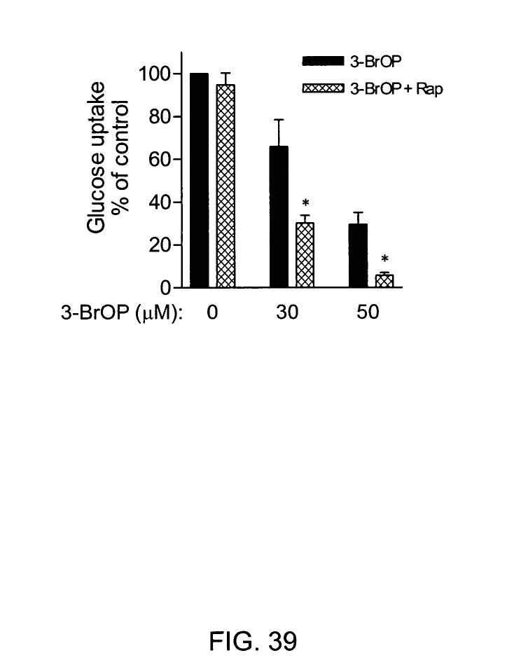

Recent studies suggest that the mTOR pathway may play an important role of in promoting cellular nutrient uptake and cell growth (Bjornstii and Houghton, 2004; Edinger and Thompson, 2002) In specific embodiments, rapamycin, by inhibiting mTOR, might compromise the ability of cellular uptake of glucose and thus enhance ATP depletion by 3-BrOP. To test this possible mechanism, [³H]-deoxyglucose was used to evaluate the effect of BrOP and rapamycin on glucose uptake. Incubation of Raji cells with 30 μM and 50 μM 3-BrOP for 2 h caused a decrease of glucose uptake by 33% and 71%, respectively (FIG. 39). This acute inhibition of glucose uptake was likely due to a direct inhibition of hexokinase by 3-BrPA leading to a failure in glucose phosphorylation and subsequent block of glucose flow into the glycolytic pathway (Xu et al., 2005; Geschwind et al., 2004).

When Raji cells were treated with rapamycin (100 ng/ml) for 20 h, there was only a slight decrease (6%) in glucose uptake (FIG. 39), suggesting that this compound alone caused minimum disturbance of glucose uptake under the experimental conditions. However, when cells were pre-treated with rapamycin for 18 h followed by incubation with 3-BrOP for 2 h, glucose uptake was severely inhibited. The degree of inhibition was significantly greater than 3-BrOP alone (FIG. 39). For instance, in the presence of rapamycin, 30 μM of 3-BrOP caused a 70% inhibition of glucose uptake, compared to a 33% inhibition by 30 μM of 3-BrOP alone. Similarly, combination of 100 ng/ml of rapamycin and 50 μM of 3-BrOP led to 94% inhibition of glucose uptake, whereas 50 μM of 3-BrOP alone caused a 71% inhibition. In specific embodiments, inhibition of mTOR by rapamycin compromised the ability of cells to uptake glucose when the glycolytic pathway was inhibited by 3-BrOP.

### Example 18

### Effect of Rapamycin and Glycolycin (3-BROP) on Phosphorylation of Molecules Downstream of MTOR

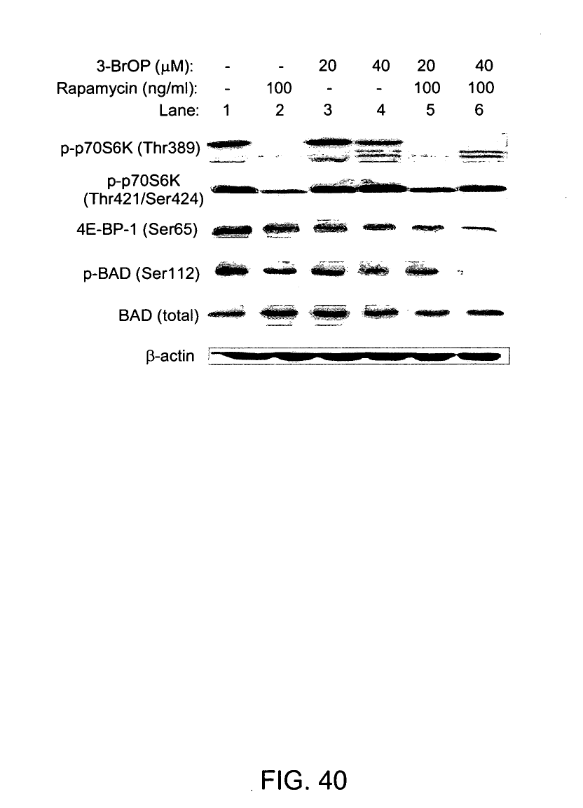

Since rapamycin at the concentration of 100 ng/ml alone did not exhibit any significant effect on the biochemical end points in the experiments described above, it is necessary to test if this concentration of rapamycin inhibit its molecular target in Raji cells. The phosphorylation of p70S6K and 4E-BP1, two target molecules of mTOR, was examined as indications of mTOR inhibition. 11, 22 As shown in FIG. 40, incubation of Raji cells with 100 ng/ml of rapamycin completely abolished the phosphorylation of p70S6K at Thr389, and a decrease in phosphorylation of p70S6K at Thr421/Ser424 and 4E-BP-1 at Ser65 (lane 2). These data suggest that this concentration of rapamycin indeed inhibited its molecular target in the cells. Interestingly, a high concentration of 3-BrOP (40 μM, lane 4) also showed some inhibition on phosphorylation of P70 S6K at Thr389 (but not at Thr421/Ser424) and 4E-BP-1 at Ser65. Combination of rapamycin and 3-BrOP appeared to cause further decrease of 4E-BP-1 phosphorylation at Ser65, and not at Thr421/Ser424 of P70 S6K.

The previous study showed that phosphorylation of BAD, a Bcl-2 family member that plays an important role in integrating glycolysis and apoptosis, was significantly decreased at Ser112 during apoptosis induced by the glycolytic inhibitor 3-BrPA (Xu et al., 2005). The effect of rapamycin on BAD phosphorylation in 3-BrOP-treated cells was examined. As illustrated in FIG. 40, the sub-toxic concentration of 3-BrOP (20 μM) did not reduce BAD phosphorylation at Ser112 (lane 3), where the toxic concentration of 3-BrOP (40 μM) caused a substantial decrease of BAD phosphorylation (lane 4). The presence of rapamycin complete abolished BAD phosphorylation in cells treated with 40 μM of 3-BrOP (lane 6), consistent with the synergistic effect of these two compounds.

In summary, the studies demonstrated that combination of rapamycin and 3-BrOP resulted in a synergistic cytotoxic effect against human lymphoma and leukemia cells, and indicate that targeting the mTOR pathway with a simultaneous inhibition of glycolysis is an effective therapeutic strategy for the treatment of hematological malignancies. Although rapamycin at a concentration of 100 ng/ml was able to inhibit the molecular target in the cells and sufficiently suppress the phosphorylation of the molecules downstream of mTOR including P70 S6K and 4E-BP-1, such inhibition of the mTOR pathway alone only led to a moderate cytostatic effect without significant cytotoxicity. In contrast, inhibition of glycolytic pathway significantly blocked ATP generation in cancer cells, which are highly dependent on glycolysis for energy supply, and produced is cytotoxic effect. Since the mTOR pathway plays an important role in promoting cellular nutrient uptake, cell growth and cell survival, combination of mTOR inhibition and blockage of glycolysis seems to be a mechanism-based strategy to severely impact cancer cell energy metabolism and effectively kill the malignant cells. Indeed, the data showed that combination of rapamycin and 3-BrOP synergistically suppressed glucose uptake and severely depleted cellular ATP pools, leading to synergistic cell killing. On the other hand, combination of rapamycin and ara-C appears not effective, and suggest that a simultaneous inhibition of mTOR pathway by rapamycin and suppression of DNA synthesis by ara-C may not lead to enhancement of cell killing. The findings indicate that the use of an mTOR inhibitor such as CCI-779 or RAD001 in combination with a glycolytic inhibitor is a useful new therapeutic strategy and in specific embodiments has significant clinical implications in treatment of cancer, including of leukemia and lymphoma.

### Example 19

### Materials and Methods for Examples 14-18

The present example provides exemplary methods and reagents to practice certain embodiments of the invention. The skilled artisan is aware that these methods and reagents may be modified without affecting the scope of the invention.

Chemicals and Reagents

Rapamycin was purchased from LC Laboratories (A division of PKC Pharmaceutical, Inc., Woburn, Mass.). 3-Bromo-2-oxopropionate and 1-propanol were purchased from Sigma (St. Loius, Mo.). 3-Bromo-2-oxopropionate propyl ester (3-BrOP) was synthesized by esterification of 3-bromo-2-oxopropionate with 1-propanol at a 1:4 molar ratio under acidic condition, followed by neutralization with sodium carbonate and evaporation of the excess 1-propanol under vacuum. [³H]-2-Deoxyglucose was purchased from Amersham (Piscataway, N.J.). Rabbit phospho-p70S6K (Thr389) antibody, phospho-p70S6K (Thr421/Ser424) antibodies, phospho-4E-BP1(Thr37/46) antibody, mouse anti-pBAD (S112), and rabbit polyclonal anti-BAD antibodies were purchased from Cell Signaling Technology, Inc (Beverly, Mass.).

Cells and Cell Cultures

Human lymphoma cell line Raji and leukemia cell line HL-60 were routinely maintain in RPMI 1640 medium supplemented with 10% fetal bovine serum (FBS) in a tissue culture incubator at 37° C. in a humidified atmosphere with 5% CO₂. The cultured cells were regularly split and maintained in the exponentially growing phase. Cells were diluted with fresh culture medium and incubated overnight before they were used in experiments.

Assays of Cell Growth and Cytotoxicity

Cell growth inhibition was determined by direct counting of the cells after they were treated with rapamycin, 3-BrOP, or their combination. A Coulter Z2 Particle Count & Size Analyzer was used to determine cell counts at 24 h, 48 h, and 72 h after drug incubation. Apoptosis was measured by flow cytometric analysis of cells stained with annexin V-FITC and propidium iodide (PI). Cells were treated with the indicated compounds under various incubation conditions as described in the figure legends. The samples were collected, washed with cold PBS, and then suspended in annexin-V binding buffer. The cells were stained with annexin V-FITC for 15 min at room temperature, washed, and then stained with PI. The samples were analyzed using a FACSCalibur flow cytometer equipped with CELLQuestPro software (Becton-Dickinson, San Jose, Calif.).

Measurement of Glucose Uptake and Cellular ATP Pool

After cells were treated with rapamycin and 3-BrOP under the desired conditions, cellular glucose uptake was measured by incubating the cells in glucose-free RPMI 1640 media with 0.2 μCi/ml of [³H]-2-deoxyglucose (specific activity, 40 Ci/mmol) for 60 min. After the samples were washed three times with cold PBS, the radioactivity in the cell pellets was quantified by liquid scintillation counting. To determine the effect of drug incubation on cellular ATP pool, cells were incubated with various concentrations of 3-BrOP and/or rapamycin for indicated time intervals. Intracellular nucleotides were extracted from the cells with 0.4 N of perchloric acid (PCA) for 10 min in ice-bath, neutralization with concentrated KOH to pH 7.0, and analyzed by HPLC analysis as described previously (Xu et al., 2005; Seymour et al., 1996). The intracellular ATP contents were calculated using the ATP standard curve generated under identical HPLC conditions, and normalized by equal cell number. The relative cellular ATP levels were expressed as percent of the control cells.

Western Blot Analysis

Protein lysates were prepared from the control and drug-treated cells, and separated by electrophoresis on 10% SDS-PAGE. After the proteins were transferred to nitrocellulose membranes, they were blotted for molecules of interest, using specific antibodies against p-p70S6K (Thr389), p-p70S6K (Thr421/Ser424), p-4E-BP1 (Thr37/46), pBAD (S112), and total BAD. The antibodies were dilute at 1:1,000 unless indicated otherwise. The bound primary antibodies were detected using appropriate horseradish peroxidase-conjugated secondary antibodies, followed by detection with a SuperSignal enhanced chemiluminescence kit (Pierce, Rockford, Ill.). For sequential blotting, the membranes were stripped with a stripping buffer (Pierce, Rockford, Ill.) followed by re-blotting with proper antibodies.

### REFERENCES

All patents and publications mentioned in the specification are indicative of the levels of those skilled in the art to which the invention pertains. All patents and publications are herein incorporated by reference in their entirety to the same extent as if each individual publication was specifically and individually indicated to be incorporated by reference.

### Patents and Patent Applications

U.S. Pat. No. 4,935,450
U.S. Pat. No. 6,472,378
U.S. Pat. No. 6,670,330
U.S. Patent Application Publication No. U.S. 2003/0013656
U.S. Patent Application Publication No. U.S. 2003/0013657
U.S. Patent Application Publication No. U.S. 2003/0013846
U.S. Patent Application Publication No. U.S. 2003/0013847
U.S. Patent Application Publication No. U.S. 2003/0139331
U.S. Patent Application Publication No. U.S. 2003/0181393
U.S. Patent Application Publication No. U.S. 2003/0087961
WO 02/45720
WO 03/105862
EP 0 273 085
EP 0 651 636

### Publications

Bangham, et al., “Diffusion of univalent Ions across the Lamellae of Swollen Phospholipids” J. Mol. Biol., 13:238-252, 1965.
Bjornsti M A, Houghton P J. The TOR pathway: a target for cancer therapy. Nat Rev Cancer 2004; 4: 335-348.
Chou T C, Hayball M P. CalcuSyn: window software for dose effect analysis. Cambridge United Kingdom: Biosoft; 1996.
Dancey J E. Inhibitors of the mammalian target of rapamycin. Expert Opin Investig Drugs 2005; 14: 313-328.
Deamer and P. Uster, “Liposome Preparation: Methods and Mechanisms,” in Liposomes (M. Ostro, ed.), Marcel Dekker, Inc., New York (1983), pp. 27-52.
Dennis, Patrick B., Jaeschke, Anja, Saitoh, Masao, Fowler, Brian, Kozma, Sara C., Thomas, George. Mammalian TOR: A Homeostatic ATP Sensor. Science 2001; 294:1102-1105.
DRUG CARRIERS IN BIOLOGY AND MEDICINE, G. Gregoriadis ed. (1979) pp. 287-341.
Easton J B, Houghton P J: Therapeutic potential of target of rapamycin inhibitors. Expert Opin Ther Targets. 2004 8: 551-564
Edinger A L, and Thompson C B. Akt Maintains Cell Size and Survival by Increasing mTOR-dependent Nutrient Uptake. Mol Biol Cell 2002 13: 2276-2288.
Fraley and Formari Kaplan, “Entrapment of a bacterial plasmid in phospholipid vesicles:potential for gene transfer,” Proc. Nat'l. Acad. Sci. USA 76:3348-3352, 1979.
Geschwind, J.-F. H. et al., Cancer Res. 62:3909-3913, 2002.
Geschwind J F, Georgiades C S, Ko Y H, Pedersen P L. Recently elucidated energy catabolism pathways provide opportunities for novel treatments in hepatocellular carcinoma. Expert Rev Anticancer Ther 2004; 4: 449-457.
Ghosh and Bachhawat, “Targeting of liposomes to hepatocytes,” In: Wu G. Wu C ed., Liver diseases, targeted diagnosis and therapy using specific receptors and ligands, New York: Marel Dekker, pp. 87-104, 1991.
Gregoriadis G. and Davis C. “Stability of liposomes in vivo and in vitro is promoted by their cholesterol content and the presence of blood cells,” Biochem Biophys Res Commun., 89(4):1287-1293, 1979.
Gregoriadis, DRUG CARRIERS IN BIOLOGY AND MEDICINE, G. Gregoriadis (ed.), 1979, pp. 287-341.
Hay N, Sonenberg N. Upstream and downstream of mTOR. Genes Dev 2004; 18:1926-1945.
Huang, L. Shu, M. Dilling, J. Easton, F. Harwood, H. Ichijo, P. Houghton. Sustained Activation of the JNK Cascade and Rapamycin-Induced Apoptosis Are Suppressed by p53/p21Cip1. Mol Cell 2003; 11: 1491-1501.
Kaneda et al., “Increased expression of DNA cointroduced with nuclear protein in adult rat liver,” Science, 243:375-378, 1989.
Kaneda et al., “Introduction and expression of the human insulin gene in adult rat liver,” J Biol. Chem., 264(21):12126-12129, 1989.
Kato et al., “Expression of hepatitis B virus surface antigen in adult rat liver. Co-introduction of DNA and nuclear protein by a simplified liposome method,” J Biol. Chem., 266(6):3361-3364, 1991.
Ko et al., Cancer Lett 173:83-91, 2001.
Ko Y H, Smith B L, Wang Y, Pomper M G, Rini D A, Torbenson M S, Hullihen J, Pedersen P L. Advanced cancers: eradication in all cases using 3-bromopyruvate therapy to deplete ATP. Biochem Biophys Res Commun 2004; 324: 269-275.
Lowe et al., Biochemistry, 23: 91-97, 1984.
Maher J C, Krishan A, Lampidis T. Greater cell cycle inhibition and cytotoxicity induced by 2-deoxy-D-glucose in tumor cells treated under hypoxic vs aerobic condition. Cancer Chemother Pharmacol 2004; 53: 116-122.
Maschek G, Savaraj N, Priebe W, et al. 2-Deoxy-D-glucose increases the efficacy of Adriamycin and paclitaxel in human osteosarcoma and non-small cell lung cancers in vivo. Cancer Res 2004; 64: 31-34.
Nicolau and Sene, “Liposome-mediated DNA transfer in eukaryotic cells: dependence of the transfer efficiency upon the type of liposomes used and the host cell cycle stage,” Biochem. Biophys. Acta, 721:185-190, 1982.
Nicolau et al., “Liposomes as carriers for in vivo gene transfer and expression,” Methods Enzymol., 149:157-176, 1987.
Panwalkar A, Verstovsek S, Giles F J. Mammalian target of rapamycin inhibition as therapy for hematologic malignancies. Cancer 2004; 100: 657-666.
Pelicano et al., J Biol. Chem. 278(39):37832-9, 2003; Epub 2003 Jul 09.
Perales et al., “Gene transfer in vivo: sustained expression and regulation of genes introduced into the liver by receptor-targeted uptake,” Proc. Natl. Acad. Sci. USA, 91:4086-4090, 1994.
Peterson R T, Desai B N, Hardwick J S, and Schreiber S L. Protein phosphatase 2A interacts with the 70-kDa S6 kinase and is activated by inhibition of FKBP12-rapamycinassociated protein. Proc Natl Acad Sci USA 1999; 96: 4438-4442.
Raymond E, Alexandre J, Faivre S, Vera K, Materman E, Boni J, Leister C, Korth-Bradley J, Hanauske A, Armand J P. Safety and Pharmacokinetics of Escalated Doses of Weekly Intravenous Infusion of CCI-779, a Novel mTOR Inhibitor, in Patients With Cancer. J Clin Oncol 2004; 22: 2336-2347.
Schmelzle T, Hall M N. TOR, a central controller of cell growth. Cell 2000; 103: 253-262.
Seymour J F, Huang P, Plunkett W, Gandhi V. Influence of fludarabine on pharmacokinetics and pharmacodynamics of cytarabine: implications for a continuous infusion schedule. Clin Cancer Res 1996; 2: 653-658.
Szoka and Papahadjopoulos, “Procedure for Preparation of Liposomes With Large Internal Aqueous Space . . . ”, Proc. Natl. Acad. Sci., 75:4194-4198, 1978.
Tunnicliff et al., Intl J Biochem, 9:249-252, 1978.
Wagner et al., Science, 260:1510-1513, 1990.
Warburg O. On the origin of cancer cells. Science 1956; 123: 309-314.
Warburg O. The metabolism of tumors. London: Constable Co. Ltd. 1930.
Wong et al., “Appearance of beta-lactamase activity in animal cells upon liposome-mediated gene transfer,” Gene., 10(2):87-94, 1980.
Wu and Wu, “Receptor-mediated in vitro gene transfections by a soluble DNA carrier system,” J. Biol. Chem., 262:4429-4432, 1987.
Wu and Wu, Adv. Drug Delivery Rev., 12:159-167, 1993.
Xu R H, Pelicano H, Zhou Y, Carew J S., Feng L, Bhalla K N., Keating M J, Huang P. Inhibition of Glycolysis in Cancer Cells: A Novel Strategy to Overcome Drug Resistance Associated with Mitochondrial Respiratory Defect and Hypoxia. Cancer Res 2005; 65: 613-621.
Zu X L, Guppy M. Cancer metabolism: facts, fantasy, and fiction. Biochem Biophys Res Commun 2004; 313: 459-465.
Zupping, J. Neurosurg. 36:451-462.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means methods, or steps.

1. A composition comprising the following general formula: 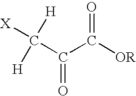
wherein X is a halogen and the composition is further characterized as follows:
(a) wherein R is a covalently bonded alkyl group comprising from one to eight carbon atoms; or
(b) wherein R is a metal ion, and wherein the composition further comprises a stabilizing agent.

2. The composition of claim 1, wherein the halogen is a bromine.

3. The composition of claim 1, wherein the alkyl group is an aliphatic group.

4. The composition of claim 3, wherein the aliphatic group is a methyl group, an ethyl group, a propyl group, a butanol group, or a pentanol group, a hexanol group, a heptanol group, or an octanol group.

5. The composition of claim 1, wherein the alkyl group is a ring structure.

6. The composition of claim 5, wherein the ring structure comprises a cycloalkanol, a benzene derivative, a steroid group with a side chain, or a steroid group without a side chain.

7. The composition of claim 1, wherein the metal ion is further defined as an alkali metal ion.

8. The composition of claim 7, wherein the alkali metal ion is sodium.

9. The composition of claim 1, wherein the stabilizing agent comprises carbonic acid.

10. The composition of claim 9, wherein the composition of (a) is further defined as sodium 3-halo-2-oxopropionate, and wherein the composition comprises hydrogen bonding between sodium 3-halo-2-oxopropionate and carbonic acid.

11. The composition of claim 1, wherein the composition of (a) is further defined as sodium 3-halo-2-oxopropionate and wherein the sodium 3-halo-2-oxopropionate and the stabilizing agent are present in a desired ratio.

12. The composition of claim 11, wherein the desired ratio is about 2:1 of sodium 3-halo-2-oxopropionate to stabilizing agent, respectively.

13. The composition of claim 1, further comprising one or more alternate energy agents.

14. The composition of claim 13, wherein said alternate energy agent is further defined as a metabolic intermediate.

15. The composition of claim 14, wherein the metabolic intermediate is a metabolic intermediate in the tricarboxylic acid (TCA) cycle, is a metabolic intermediate in mitochondrial respiration, is a metabolic intermediate in both the TCA cycle and mitochondrial respiration, is a precursor to the TCA cycle, is a precursor of a metabolic intermediate in mitochondrial respiration, or is a precursor of a metabolic intermediate in both the TCA cycle and mitochondrial respiration.

16. The composition of claim 14, wherein said metabolic intermediate comprises glutamine, pyruvate, a fatty acid, or a combination thereof.

17. The composition of claim 16, wherein the fatty acid comprises an alkyl chain having no double bonds.

18. The composition of claim 16, wherein the fatty acid comprises an alkyl chain having one or more double bonds.

19. The composition of claim 1, further defined as being comprised in a pharmaceutical formulation.

20. A method for inhibiting glycolysis in a cell, comprising delivering to the cell a composition of claim 1.

21. The method of claim 20, wherein said method is further defined as inducing apoptosis or necrosis in said cell or inhibiting proliferation in said cell.

22. The method of claim 20, wherein said cell is a cancer cell.

23. The method of claim 22, wherein said cancer cell is comprised in an individual.

24. The method of claim 22, wherein said cancer cell is in a solid tumor.

25. The method of claim 22, wherein said cancer cell is a leukemia cell, breast cancer cell, lung cancer cell, prostate cancer cell, pancreatic cancer cell, colon cancer cell, head and neck cancer cell, liver cancer cell, bone cancer cell, ovarian cancer cell, cervical cancer cell, spleen cancer cell, brain cancer cell, esophageal cancer cell, lymphoma cell, or skin cancer cell.

26. The method of claim 22, wherein said cancer cell is a drug-resistant cancer cell.

27. The method of claim 26, wherein said drug-resistant cancer cell is a leukemia cell.

28. The method of claim 22, wherein said cancer cell is in a hypoxic environment.

29. A method of treating cancer in an individual, comprising the step of administering to the individual a therapeutically effective amount of a composition of claim 1.

30. The method of claim 29, further comprising administering to the individual an additional cancer therapy.

31. The method of claim 30, wherein the additional cancer therapy comprises radiation, chemotherapy, surgery, gene therapy, immunotherapy, hormone therapy, or a combination thereof.

32. The method of claim 31, wherein the additional cancer therapy comprises radiation.

33. The method of claim 30, wherein the additional cancer therapy comprises a drug.

34. The method of claim 33, wherein the drug is an inhibitor of the mammalian target of rapamycin (mTOR) pathway.

35. The method of claim 30, wherein the additional cancer therapy is administered to the individual prior to the administration of the composition of claim 1, concomitant with the administration of the composition of claim 1, subsequent to the administration of the composition of claim 1, or a combination thereof.

36. The method of claim 29, wherein at least some of the cancer of the individual resides in a hypoxic environment.

37. The method of claim 36, wherein the hypoxic environment is further defined as being in a solid tumor.

38. The method of claim 29, wherein the cancer is leukemia.

39. The method of claim 29, wherein the cancer is brain cancer, lung cancer, breast cancer, prostate cancer, pancreatic cancer, ovarian cancer, liver cancer, bone cancer, stomach cancer, esophageal cancer, colon cancer, head and neck cancer, leukemia, lymphoma, melanoma, spleen cancer, cervical cancer, kidney cancer, or throat cancer.

40. A kit comprising a composition of claim 1 housed in a suitable container.

41. The kit of claim 40, wherein the composition of (a) comprises sodium 3-bromo-2-oxopropionate.

42. The kit of claim 41, wherein the sodium 3-bromo-2-oxopropionate and the stabilizing agent are housed in the same container.

43. The kit of claim 41, wherein the sodium 3-bromo-2-oxopropionate and the stabilizing agent are housed in separate containers.

44. The kit of claim 40, wherein the container comprises sterilized CO₂ gas in its void volume.

45. The kit of claim 40, further comprising a pharmaceutically acceptable diluent.
